SEQUENCE LISTING (116 sequences)

SEQ ID NO: 1 - protein, 936 aa, artificial sequence
  HBV1530_T01.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 2 - protein, 942 aa, artificial sequence
  HBV1530_T01.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 3 - protein, 936 aa, artificial sequence
  HBV1860_T01.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 4 - protein, 942 aa, artificial sequence
  HBV1860_T01.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 5 - protein, 936 aa, artificial sequence
  HBV2400_T01.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 6 - protein, 942 aa, artificial sequence
  HBV2400_T01.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 7 - protein, 936 aa, artificial sequence
  HBV180_T01.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 8 - protein, 942 aa, artificial sequence
  HBV180_T01.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 9 - protein, 1065 aa, artificial sequence
  HBV-core-1.L1
MGDPKKKRKVIDYPYDVPDYAIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 10 - protein, 1071 aa, artificial sequence
  HBV-core-1.R1
MGDPKKKRKVIDKETAAAKFERQHMDSIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 11 - protein, 1065 aa, artificial sequence
  HBV-core-2.L1
MGDPKKKRKVIDYPYDVPDYAIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 12 - protein, 1071 aa, artificial sequence
  HBV-core-2.R1
MGDPKKKRKVIDKETAAAKFERQHMDSIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 13 - protein, 936 aa, artificial sequence
  HBV-polym-1.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 14 - protein, 942 aa, artificial sequence
  HBV-polym-1.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 15 - protein, 1065 aa, artificial sequence
  HBV-polym-2.L1
MGDPKKKRKVIDYPYDVPDYAIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 16 - protein, 1071 aa, artificial sequence
  HBV-polym-2.R1
MGDPKKKRKVIDKETAAAKFERQHMDSIDIADPIRSRTPSPARELLPGPQPDGVQPTADRGVSPPAGGPLDGLPARRTMSRTRLPSPPAPSPAFSAGSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 17 - protein, 936 aa, artificial sequence
  HBV-HBX-1.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 18 - protein, 942 aa, artificial sequence
  HBV-HBX-1.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 19 - protein, 936 aa, artificial sequence
  HBV-HBX-2.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 20 - protein, 942 aa, artificial sequence
  HBV-HBX-2.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 21 - protein, 936 aa, artificial sequence
  mm_F7_exon1-3.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 22 - protein, 942 aa, artificial sequence
  mm_F7_exon1-3.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 23 - protein, 913 aa, artificial sequence
  mm_F7_exon6-2.L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 24 - protein, 919 aa, artificial sequence
  mm_F7_exon6-2.R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 25 - protein, 936 aa, artificial sequence
  hAPOC3_Ex3A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 26 - protein, 942 aa, artificial sequence
  hAPOC3_Ex3A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 27 - protein, 936 aa, artificial sequence
  hAPOC3_Ex3B-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 28 - protein, 942 aa, artificial sequence
  hAPOC3_Ex3B-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 29 - protein, 941 aa, artificial sequence
  hAPOC3_Ex3_AN-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 30 - protein, 936 aa, artificial sequence; identical to SEQ ID NO: 25
  hAPOC3_Ex3A-L1

SEQ ID NO: 31 - protein, 936 aa, artificial sequence
  hTTR_Ex1A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 32 - protein, 942 aa, artificial sequence
  hTTR_Ex1A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 33 - protein, 936 aa, artificial sequence
  hTTR_Ex1B-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 34 - protein, 942 aa, artificial sequence
  hTTR_Ex1B-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 35 - protein, 913 aa, artificial sequence
  hSMN2_3ssEx8A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 36 - protein, 919 aa, artificial sequence
  hSMN2_3ssEx8A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPSGSGSGGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 37 - protein, 936 aa, artificial sequence
  hSMN2_3ssEx8B-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 38 - protein, 942 aa, artificial sequence
  hSMN2_3ssEx8B-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 39 - protein, 936 aa, artificial sequence
  hSMN2_ISS100A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 40 - protein, 942 aa, artificial sequence
  hSMN2_ISS100A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 41 - protein, 936 aa, artificial sequence
  hSMN2_ISS-N1A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 42 - protein, 942 aa, artificial sequence
  hSMN2_ISS-N1A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 43 - protein, 936 aa, artificial sequence
  hIDOL_Ex2A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 44 - protein, 942 aa, artificial sequence
  hIDOL_Ex2A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 45 - protein, 936 aa, artificial sequence
  hIDOL_Ex3A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 46 - protein, 942 aa, artificial sequence
  hIDOL_Ex3A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 47 - protein, 936 aa, artificial sequence
  hANGPTL3_Ex1A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 48 - protein, 942 aa, artificial sequence
  hANGPTL3_Ex1A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 49 - protein, 936 aa, artificial sequence
  hANGPTL3_Ex2A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 50 - protein, 942 aa, artificial sequence
  hANGPTL3_Ex2A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 51 - protein, 936 aa, artificial sequence
  hPCSK9_Ex3A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 52 - protein, 942 aa, artificial sequence
  hPCSK9_Ex3A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 53 - protein, 936 aa, artificial sequence
  hPCSK9_Ex12A-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 54 - protein, 942 aa, artificial sequence
  hPCSK9_Ex12A-R1
MGDPKKKRKVIDKETAAAKFERQHMDSIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 55 - protein, 935 aa, artificial sequence
  hPCSK9_Ex3_AN-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 56 - protein, 942 aa, artificial sequence; identical to SEQ ID NO: 52
  hPCSK9_Ex3A-R1

SEQ ID NO: 57 - protein, 942 aa, artificial sequence; identical to SEQ ID NO: 54
  hPCSK9_Ex12A-R1

SEQ ID NO: 58 - protein, 935 aa, artificial sequence
  hPCSK9Ex12_AN-L1
MGDPKKKRKVIDYPYDVPDYAIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 59 - DNA, 16 bp, artificial sequence
  HBV1530_T01.L1
ctgtgccttctcatct

SEQ ID NO: 60 - DNA, 16 bp, artificial sequence
  HBV1530_T01.R1
gcagaggtgaagcgat

SEQ ID NO: 61 - DNA, 16 bp, artificial sequence
  HBV1860_T01.L1
gttcaagcctccaagt

SEQ ID NO: 62 - DNA, 16 bp, artificial sequence
  HBV1860_T01.R1
gtccatgccccaaagt

SEQ ID NO: 63 - DNA, 16 bp, artificial sequence
  HBV2400_T01.L1
cgcagaagatctcaat

SEQ ID NO: 64 - DNA, 16 bp, artificial sequence
  HBV2400_T01.R1
ccaagggatactaact

SEQ ID NO: 65 - DNA, 16 bp, artificial sequence
  HBV180_T01.L1
gttacaggcggggttt

SEQ ID NO: 66 - DNA, 16 bp, artificial sequence
  HBV180_T01.R1
gtggtattgtgaggat

SEQ ID NO: 67 - DNA, 16 bp, artificial sequence
  HBV-core-1.L1
gtggattcgcactcct

SEQ ID NO: 68 - DNA, 16 bp, artificial sequence
  HBV-core-1.R1
aggggcatttggtggt

SEQ ID NO: 69 - DNA, 16 bp, artificial sequence
  HBV-core-2.L1
gcccctatcttatcat

SEQ ID NO: 70 - DNA, 16 bp, artificial sequence
  HBV-core-2.R1
cgtctaacaacagtat

SEQ ID NO: 71 - DNA, 16 bp, artificial sequence
  HBV-polym-1.L1
gtctgcggcgttttat

SEQ ID NO: 72 - DNA, 16 bp, artificial sequence
  HBV-polym-1.R1
gaggcatagcagcagt

SEQ ID NO: 73 - DNA, 16 bp, artificial sequence
  HBV-polym-2.L1
ccctcgcctcgcagat

SEQ ID NO: 74 - DNA, 16 bp, artificial sequence
  HBV-polym-2.R1
cttctgcgacgcggct

SEQ ID NO: 75 - DNA, 16 bp, artificial sequence
  HBV-HBX-1.L1
tctcatctgccggtct

SEQ ID NO: 76 - DNA, 16 bp, artificial sequence
  HBV-HBX-1.R1
gcaacgtgcagaggtt

SEQ ID NO: 77 - DNA, 16 bp, artificial sequence
  HBV-HBX-2.L1
ttacgcggtctcccct

SEQ ID NO: 78 - DNA, 16 bp, artificial sequence
  HBV-HBX-2.R1
gcacacggaccggcat

SEQ ID NO: 79 - DNA, 16 bp, artificial sequence
  mm_F7_exon1-3.L1
ctctgctttctgctct

SEQ ID NO: 80 - DNA, 16 bp, artificial sequence
  mm_F7_exon1-3.R1
atacctgcagtccctt

SEQ ID NO: 81 - DNA, 16 bp, artificial sequence
  mm_F7_exon6-2.L1
ctctctgacttctgtt

SEQ ID NO: 82 - DNA, 16 bp, artificial sequence
  mm_F7_exon6-2.R1
tctcccacacgggtat

SEQ ID NO: 83 - DNA, 16 bp, artificial sequence
  hAPOC3_Ex3A-L1
ctgttgcttcccctgt

SEQ ID NO: 84 - DNA, 16 bp, artificial sequence
  hAPOC3_Ex3A-R1
gaagccatcggtcact

SEQ ID NO: 85 - DNA, 16 bp, artificial sequence
  hAPOC3_Ex3B-L1
gaaagactactggagt

SEQ ID NO: 86 - DNA, 16 bp, artificial sequence
  hAPOC3_Ex3B-R1
cccagaactcagagat

SEQ ID NO: 87 - DNA, 15 bp, artificial sequence
  hAPOC3_Ex3_AN-R1
gaagccatggtcact

SEQ ID NO: 88 - DNA, 16 bp, artificial sequence
  hAPOC3_Ex3A-L1
ctgttgcttcccctgt

SEQ ID NO: 89 - DNA, 16 bp, artificial sequence
  hTTR_Ex1A-L1
cttggcaggatggctt

SEQ ID NO: 90 - DNA, 16 bp, artificial sequence
  hTTR_Ex1A-R1
ccagcaaggcagaggt

SEQ ID NO: 91 - DNA, 16 bp, artificial sequence
  hTTR_Ex1B-L1
gtctgaggctggccct

SEQ ID NO: 92 - DNA, 16 bp, artificial sequence
  hTTR_Ex1B-R1
aggaatgggatgtcat

SEQ ID NO: 93 - DNA, 16 bp, artificial sequence
  hSMN2_3ssEx8A-L1
ctggttctaatttctt

SEQ ID NO: 94 - DNA, 16 bp, artificial sequence
  hSMN2_3ssEx8A-R1
gctgctctatgccagt

SEQ ID NO: 95 - DNA, 16 bp, artificial sequence
  hSMN2_3ssEx8B-L1
ggttctaatttctcat

SEQ ID NO: 96 - DNA, 16 bp, artificial sequence
  hSMN2_3ssEx8B-R1
tagtgctgctctatgt

SEQ ID NO: 97 - DNA, 16 bp, artificial sequence
  hSMN2_ISS100A-L1
tcagatgttagaaagt

SEQ ID NO: 98 - DNA, 16 bp, artificial sequence
  hSMN2_ISS100A-R1
ttaatattgattgttt

SEQ ID NO: 99 - DNA, 16 bp, artificial sequence
  hSMN2_ISS-N1A-L1
taaggagtaagtctgt

SEQ ID NO: 100 - DNA, 16 bp, artificial sequence
  hSMN2_ISS-N1A-R1
tacaaaagtaagattt

SEQ ID NO: 101 - DNA, 16 bp, artificial sequence
  hIDOL_Ex2A-L1
ttatggctaaacctgt

SEQ ID NO: 102 - DNA, 16 bp, artificial sequence
  hIDOL_Ex2A-R1
agcccatccatctgct

SEQ ID NO: 103 - DNA, 16 bp, artificial sequence
  hIDOL_Ex3A-L1
gccaaggagctctcct

SEQ ID NO: 104 - DNA, 16 bp, artificial sequence
  hIDOL_Ex3A-R1
attcaacagcctcact

SEQ ID NO: 105 - DNA, 16 bp, artificial sequence
  hANGPTL3_Ex1A-L1
attgttcctctagttt

SEQ ID NO: 106 - DNA, 16 bp, artificial sequence
  hANGPTL3_Ex1A-R1
gatgaattgtcttgat

SEQ ID NO: 107 - DNA, 16 bp, artificial sequence
  hANGPTL3_Ex2A-L1
ccagacttttgtagat

SEQ ID NO: 108 - DNA, 16 bp, artificial sequence
  hANGPTL3_Ex2A-R1
ggagaaggtctttgat

SEQ ID NO: 109 - DNA, 16 bp, artificial sequence
  hPCSK9_Ex3A-L1
tgccccatgtcgactt

SEQ ID NO: 110 - DNA, 16 bp, artificial sequence
  hPCSK9_Ex3A-R1
ctgggcaaagacagat

SEQ ID NO: 111 - DNA, 16 bp, artificial sequence
  hPCSK9_Ex12A-L1
ggcaggtgaccgtggt

SEQ ID NO: 112 - DNA, 16 bp, artificial sequence
  hPCSK9_Ex12A-R1
gcagccagtcagggtt

SEQ ID NO: 113 - DNA, 15 bp, artificial sequence
  hPCSK9_Ex3_AN-L1
tgccccatgtgactt

SEQ ID NO: 114 - DNA, 16 bp, artificial sequence
  hPCSK9_Ex3A-R1
ctgggcaaagacagat

SEQ ID NO: 115 - DNA, 16 bp, artificial sequence
  hPCSK9_Ex12A-R1
gcagccagtcagggtt

SEQ ID NO: 116 - DNA, 15 bp, artificial sequence
  hPCSK9Ex12_AN-L1
ggcaggtgacgtggt

(19) United States
(12) Patent Application Publication
(10) Pub. No.: US 2021/0139870 A1
(43) Pub. Date: May 13, 2021

(54) ANTI-HBV COMBINATION THERAPIES INVOLVING SPECIFIC ENDONUCLEASES

(71) Applicant: CELLECTIS, PARIS (FR)

(72) Inventor: Philippe DUCHATEAU, DRAVEIL (FR)

(21) Appl. No.: 16/623,171

(22) PCT Filed: Jun. 18, 2018
(86) PCT No.: PCT/EP2018/066110

§ 371 Date: Dec. 16, 2019

(30) Foreign Application Priority Data
Jun. 19, 2017 (DK) PA201770478

(51) Int. Cl. C12N 9/22; A61K 45/06; A61K 47/55; A61P 31/22

(52) U.S. Cl. CPC C12N 9/22; A61K 45/06; C12Y 301/00; A61P 31/22; A61K 47/55

(57) ABSTRACT

The invention pertains to non-viral methods for in vivo delivery of endonuclease reagents compositions to specific tissues or cells. According to the invention, the endonuclease reagents are preferably encapsulated into micelle structures of 50 to 150 nm diameter for intravenous injection, in combination with antiviral compounds. The invention thereby provides therapeutic compositions comprising endonuclease reagents and antiviral compounds for effective elimination of HBV from liver cells and treatment of chronic hepatitis.

### FIELD OF THE INVENTION

The invention pertains to therapies involving specific TAL-nucleases against hepatitis B virus (HBV), and more specifically to non-viral methods for in vivo delivery of such TAL-nucleases to specific tissues or cells. The invention thereby provides therapeutic compositions, by which HBV specific endonuclease reagent can be released into liver cells, preferably under RNA form, in combination with other antiviral compounds.

### BACKGROUND OF THE INVENTION

The potential of gene editing in various therapies has long been envisioned by the applicant (WO2004067753), especially to repair or modulate the expression of deficient genes causing genetic disease, or to knock-out deleterious genetic sequences, either sexually inherited or introduced into the cells by infectious agents.

Since the emergence of the first programmable meganuclease reagents by the turn of the century (Smith et al. (2006) A combinatorial approach to create artificial homing endonucleases cleaving chosen sequences. Nucleic Acids Res. 34(22):e149), endonucleases reagents have rapidly evolved, offering improved specificity, safety and reliability. In particular, TALE-nucleases, which are fusions of a TALE binding domain with a cleavage catalytic domain (WO2011072246) have proven to be highly specific for therapeutic purposes (Leukaemia success heralds wave of gene-editing therapies (2015) Nature 527:146-147). TALE-nucleases are particularly specific when they are used by pairs under obligatory heterodimeric form, by using the dimeric cleavage domain of Fok-1. Left and right heterodimer members each recognizes a different nucleic sequences of about 14 to 20 bp, together spanning target sequences of 30 to 50 bp overall specificity.

More recently, further endonucleases reagents have been developed based on the components of the type II prokaryotic CRISPR (Clustered Regularly Interspaced Short palindromic Repeats) adaptive immune system of the bacteria S. pyogenes. This multi-component system referred to as RNA-guided nuclease system (Gasiunas, Barrangou et al. 2012; Jinek, Chylinski et al. 2012), involves members of Cas9 or Cpf1 endonuclease families coupled with a guide RNA molecules that have the ability to drive said nuclease to some specific genome sequences (Zetsche et al. (2015). Cpf1 is a single RNA-guided endonuclease that provides immunity in bacteria and can be adapted for genome editing in mammalian cells. Cell 163:759-771). Such programmable RNA-guided endonucleases are easy to produce because the cleavage specificity is conferred by the sequence of the guide RNA, which can be cheaply adapted. Their specificity although stands on shorter sequences than TAL-nucleases of about 10 pb, which must be located near a particular motif (PAM) in the targeted genetic sequence.

Various proofs of concept of the efficiency and safety of the above specific endonuclease reagents have been reported in human cells in-vitro or ex-vivo, but the delivery of the same reagents into the body has still to be very carefully considered due to the risk of off-site mutations inherent to such reagents. This risk increases when the reagents, which are mainly proteins, are being degraded in-vivo, altering their specificity and cleavage properties.

The primary challenge for gene therapy is thus to develop a method that delivers a therapeutic gene (e.g. transgene) or sequence specific reagent (e.g. nuclease) to selected cells where proper gene expression can be achieved. An ideal gene delivery method needs to meet 3 major criteria: (1) it should protect the transgene or reagent against degradation by nucleases in intercellular matrices, (2) it should bring the transgene or reagent across the plasma membrane and into the nucleus of target cells, and (3) it should have no detrimental effects (Gao, X. et al. (2007) Non-viral Gene Delivery: What we know and What is next. APPS Journal. 9(1) Article 9:92-104).

Viral vectors are able to mediate gene transfer with high efficiency and the possibility of long-term gene expression, may satisfy 2 out of 3 criteria. However, the acute immune response, immunogenicity, and insertion mutagenesis uncovered in gene therapy clinical trials have raised serious safety concerns about some commonly used viral vectors.

The inventors have explored safer means for in-vivo delivery and more particularly of endonucleases reagents, which could be used to target specific tissues into the human body especially delivery of sequence specific TAL-nucleases and Cas9/CRISPR into liver cells. They have determined that encapsulating nuclease reagent under RNA form in micelle structures of 50 to 100 nm was particularly appropriate to deliver the reagents into the nucleus of the cells by intravenous injection, in efficient amount and with limited off-site effects. This was primarily demonstrated by targeting cccDNA of HBV into liver cells, but this strategy has since been proven to be expandable to various type of cells by anchoring specific cell surface protein receptors or ligand into the micelle structures, as further detailed herein.

In addition, the inventors have combined TALE-nucleases with certain antiviral drugs to produce more potent anti-HBV therapeutic compositions.

### SUMMARY OF THE INVENTION

The present invention is drawn to a method for encapsulating an endonuclease reagent, wherein said endonuclease reagent is prepared under RNA form and complexed with at least one biodegradable matrix comprising at least a core hydrophobic domain and a proximal polar domain to form particles of 50 to 100 nm diameter range, suitable for in-vivo injection.

The endonuclease reagent is generally a RNA molecule coding for a sequence-specific endonuclease reagent, such as a homing endonuclease, a zing finger nuclease, or a TALE-Nuclease, or a RNA guide optionally co-delivered with a RNA-guided endonuclease, such as cas9 or Cpf1.

Various types of biodegradable delivery capsules comprising RNA endonuclease reagents can be manufactured, depending on the structure of the biodegradable matrices involved and the monomers forming said core hydrophobic domain and polar domains.

Such biodegradable delivery capsules according to the invention are useful to deliver endonuclease reagent into the cells under RNA form, especially when co-delivery of different endonuclease reagents is sought, like for instance, messenger RNAs encoding right and left heterodimer TALE-nucleases.

Delivery specificity can be improved by linking a targeting domain to the proximal polar domain of said biodegradable matrix, such that the delivery capsules of the invention can bind surface antigens of different cell types. The delivery capsules are particularly suited for intravenous injection to target endogenous genetic sequences into cells.

The present application more particularly claims pharmaceutical compositions comprising the biodegradable delivery capsules of the invention into treatments involving endonuclease reagents. Such treatments may be part of a gene therapy, where specific genetic sequences have to be knocked-out or repaired, of an anti-infection therapy, by targeting the genome of infectious agents, or cancer therapy. The biodegradable delivery capsules of the present invention have proven to be particularly adapted to treat infectious agents that present a DNA intermediate in the liver cells, such as the cccDNA (covalently closed circular DNA) of Hepadnavirus, in particular HBV (Hepatitis B Virus), which are resistant forms of these viruses lodged into hepatocytes.

The present invention also provides with specific TALE-nucleases to target the various forms of HBV genome in liver cells to be involved into combination therapies with other anti-HBV compounds to block the virus propagation at different levels: viral load, transcription, replication and expression, HBV entry into cells, capsid assembly, and patient's immune response.

In particular, the invention provides combining the specific TALE-nucleases of the present invention with at least one of the following agents:
Inhibitor of sodium taurocholate cotransporting polypeptide (NTCP), such as Myrcludex;
cccDNA inhibitor, such as disubstituted sulfonamide (DSS) compounds, antibodies inducing Lymphotoxin beta receptor activation or LTβR agonists;
RNAi or compounds aiming at reducing HBV genes expression, in particular Helioxanthin or Ethanol extract from Ampelopsis sinica root;
La protein inhibitor, such as HBSC11;
Capsid allosteric modulators, such as ABI H0731, JNJ 56136379 (or JNJ379), Morphothiadine (GLS4), NVR 3 778 or NVR1221;
Reverse transcriptase inhibitors, in particular nucleoside analogs, such as Lamivudine, Telbivudine, Entecavir, Adefovir, Tenofovir, Besifovir, MIV-210, MCC-478 or Alamifovir;
Inhibition of HBsAg release, such as REP2139 and GC1102;
Immunoregulators, such as Thymosin-α1;
Vesatolimod (GS-9620);
Vaccines, such as GS-4774, ABX-203, TG-1050, INO-1800; FP-02.2:
Immune stimulators, such as SB-9200, AIC649, Cellular inhibitor of apoptosis proteins (cIAPs) Cytokines: IL-21 or IL-7 and antibodies antagonist of Immune checkpoint (Nivolumab and Pembrolizumab).

The resulting therapeutic compositions, which may be delivered using nanoparticles or the micelle-based approaches mentioned above, allow to dramatically reduce viral loads and more particularly the onset of chronic hepatitis.

### BRIEF DESCRIPTION OF THE FIGURES AND TABLES

FIG. 1: Schematic representation of micelle-based system according to the invention: micelles can be obtained by mixing structures A, B, C, D (described here after) to form particles of 50-100 nm diameter, by optionally incorporating protein E for extra targeting specificity. E is a protein or fusion protein containing a hydrophobic domain (for ex. derived from transmembrane protein) to anchor said protein within the micelle linked to a binding domain for endocytic receptor. The nuclease reagents are complexed to the micelles within the hydrophilic domains of A, B, C or D matrices.

A and B: Structures comprising a distal hydrophilic domain conjugated to a hydrophobic domain, itself conjugated to a proximal hydrophilic domain complexed with the endonuclease reagents under RNA form. In A, said proximal hydrophilic domain is optionally linked to an external binding protein, such as N-acetylgalactosamine. These structures self-assemble under micelles that harbor a central hydrophilic pocket.

C and D: Simpler structures consisting of a hydrophobic domain conjugated to the proximal hydrophilic domain, which is complexed with the endonuclease reagents under RNA form. In A, said proximal hydrophilic domain is optionally linked to an external binding protein, such as N-acetylgalactosamine.

FIG. 2: Schematic representation of the encapsulation of CRISPR based endonuclease reagents to perform gene editing in vivo according to the present invention. A: the guided endonuclease (ex: Cas9) is trapped into the inner hydrophilic core of the micelle, whereas the RNA guide is complexed into the polar domain of the matrix. B: the guided endonuclease (ex: Cas9) is first complexed with the RNA-guide to give a RNP (RiboNucleoProtein) that is complexed as such into the polar domain of the matrix.

FIG. 3: Schematic representation of the encapsulation heterodimeric TAL-nucleases endonuclease reagents to perform gene editing in vivo according to the present invention.

FIG. 4: Schematic representation of HBV genome cloned into cGPS HEK293 showing the position of the TALE-nucleases of the present invention.

FIG. 5: T7 endonuclease assays on TALEN set 1 (T002559 to T002564) chromatography gels and interpretation (Table 3)

FIG. 6: T7 endonuclease assays on TALEN set 2 (T001212 to T001215) chromatography gels and interpretation (Table 4)

Table 1: Exemplary list of target genes that can be modified by the gene editing method according to the invention and their associated diseases.

Table 2: Engineered TAL-nucleases used in the in-vivo gene editing method to target HBV (cccDNA) and the genes encoding respectively APOC3, TTR, SMN2, IDOL, ANGPTL3, IDOL and PCSK9 (detailed target and polypeptide sequences are provided in Table 7).

Table 3 (FIG. 5): Results of T7 for the HBV TALENs of set 1.

Table 4 (FIG. 6): Results of T7 for the HBV TALENs of set 2.

Table 5: Quantitation of the PCR bands with Biorad Lab Image program obtained upon cleavage with the HBV TALENs of the present invention.

Table 6: Summary of the mice treatment schedule to target factor VII gene in the liver.

Table 7: TALE-nucleases engineered to target the HBV genome, APOC3, TTR, SMN2, IDOL, ANGPTL3 and PCSK9 genes, along with their polypeptide sequences et target polynucleotide sequences.

TABLE 1
Exemplary list of target genes that can be modified in vivo by gene editing and
the associated disease that can be treated according to the invention

| | | NCBI | |
|---|---|---|---|
| Target Gene | Symbol | gene ID | Disease |
| proprotein convertase | PCSK9 | 255738 | (Familial) hypercholesterolemia |
| subtilisin/kexin type 9 | | | |
| apolipoprotein C-III | APOC3 | 345 | (Familial) |
| | | | hypertriglyceridemia/dyslipidemia |
| angiopoietin-like 3 | ANGPTL3 | 27329 | Combined hyperlipidemia/familial |
| | | | mixed hyperlipidemia |
| Inducible degrader of the LDLR, | MYLIP | 29116 | (Familial) hypercholesterolemia |
| IDOL | | | |
| transthyretin | TTR | 7276 | Transthyretin (TTR)-mediated |
| | | | amyloidosis (ATTR) |
| hydroxyacid oxidase (glycolate | HAO1 | 54363 | Primary hyperoxaluria type 1 (PH1) |
| oxidase) 1 | | | |
| serpin peptidase inhibitor, clade A | SERPINA1 | 5265 | Alpha 1-antitrypsin deficiency/COPD |
| transmembrane protease, serine 6 | TMPRSS6 | 164656 | Hemochromatosis and |
| | | | β-thalassemia |
| serpin peptidase inhibitor, clade C | SERPINC1 | 462 | Haemophilia |
| (antithrombin) | | | |
| solute carrier family 30 | SLC30A8 | 169026 | Diabetes mellitus type 2 |
| hepatitis B Virus | HBV | N.A. | Hepatitis - liver cancer |
| hungtingtin | HTT | 3064 | Huntington disease |
| myostatin | MSTN | 2660 | muscular degeneration |
| dystrophin | DMD | 13405 | Duchene muscular dystrophy |
| beta-2-microglobulin | B2M | 567 | graft |
| Cytomegalovirus | CMV | N.A. | Infectious disease |
| Herpes simplex Virus | HSV | N.A. | Infectious disease |
| Cystic Fibrosis Transmembrane | CFTR | 1080 | Cystic fibrosis |
| Conductance Regulator | | | |
| survival of motor neuron 2 | SMN2 | 6607 | Spinal muscular dystrophy |
| nicotinamide N-methyltransferase | NNMT | 4837 | obesity |
| chromosome 9 open reading frame | C9ORF72 | 203228 | Amyotrophic lateral sclerosis |
| 72 | | | |
| farnesyl-diphosphate | FDFT1 | 2222 | (Familial) hypercholesterolemia |
| farnesyltransferase 1 | | | |
| NPC1-like 1 | NPC1L1 | 29881 | (Familial) hypercholesterolemia |
| 3-hydroxy-3-methylglutaryl-CoA | HMGCR | 3156 | (Familial) hypercholesterolemia |
| reductase | | | |
| apolipoprotein B | APOB | 338 | (Familial) hypercholesterolemia |
| microsomal triglyceride transfer | MTTP | 4547 | (Familial) hypercholesterolemia |
| protein | | | |
| diacylglycerol O-acyltransferase 1 | DGAT1 | 8694 | (Familial) hypercholesterolemia |
| Hepatitis D virus | HDV | N.A. | infectious disease |
| Programmed death-ligand 1 | CD274 | 29126 | infectious disease |
| Fibroblast Growth Factor | FGFR4 | 2264 | obesity |
| Receptor 4 | | | |
| microRNA let-7 | let-7 | | cancer |
| microRNA miR-21 | miR-21 | | cancer |
| microRNA mir-26 | mir-26 | | cancer |
| microRNA mir-10 | mir-10 | | cancer |
| microRNA mir-34 | mir-34 | | cancer |
| microRNA mir-122 | mir-122 | | infectious disease |

TABLE 2
Engineered TAL-nucleases used in the in-vivo gene editing method

| Target | Associated | TALEN ® | SEQ ID | |
|---|---|---|---|---|
| gene | disease(s) | name | NO: # | Target sequence |
| HBV | Hepatitis - | T001212 | 1 | HBV1530_T0L.L1 |
| | liver cancer | T001212 | 2 | HBV1530_T01.R1 |
| | | T001213 | 3 | HBV1860_T01.L1 |
| | | T001213 | 4 | HBV1860_T01.R1 |
| | | T001214 | 5 | HBV2400_T01.L1 |
| | | T001214 | 6 | HBV2400_T01.R1 |
| | | T001215 | 7 | HBV180_T01.L1 |
| | | T001215 | 8 | HBV180_T01.R1 |
| | | T002559 | 9 | HBV-core-1.L1 |
| | | T002559 | 10 | HBV-core-1.R1 |
| | | T002560 | 11 | HBV-core-2.L1 |
| | | T002560 | 12 | HBV-core-2.R1 |
| | | T002561 | 13 | HBV-polym-1.L1 |
| | | T002561 | 14 | HBV-polym-1.R1 |
| | | T002562 | 15 | HBV-polym-2.L1 |
| | | T002562 | 16 | HBV-polym-2.R1 |
| | | T002563 | 17 | HBV-HBX-1.L1 |
| | | T002563 | 18 | HBV-HBX-1.R1 |
| | | T002564 | 19 | HBV-HBX-2.L1 |
| | | T002564 | 20 | HBV-HBX-2.R1 |
| APOC3 | (Familial) | T002657 | 25 | hAPOC3_Ex3A-L1 |
| | hyper- | T002657 | 26 | hAPOC3_Ex3A-R1 |
| | triglyceridemia/ | T002658 | 27 | hAPOC3_Ex3B-L1 |
| | dyslipidemia | T002658 | 28 | hAPOC3_Ex3B-R1 |
| | | T002671 | 29 | hAPOC3_Ex3_AN-R1 |
| | | T002671 | 30 | hAPOC3_Ex3A-L1 |
| TTR | Transthyretin | T002659 | 31 | hTTR_Ex1A-L1 |
| | (TTR)- | T002659 | 32 | hTTR_Ex1A-R1 |
| | mediated | T002660 | 33 | hTTR_Ex1B-L1 |
| | amyloidosis | T002660 | 34 | hTTR_Ex1B-R1 |
| | (ATTR) | | | |
| SMN2 | Spinal | T002661 | 35 | hSMN2_3ssEx8A-L1 |
| | muscular | T002661 | 36 | hSMN2_3ssEx8A-R1 |
| | dystrophy | T002662 | 37 | hSMN2_3ssEx8B-L1 |
| | | T002662 | 38 | hSMN2_3ssEx8B-R1 |
| | | T002663 | 39 | hSMN2_ISS100A-L1 |
| | | T002663 | 40 | hSMN2_ISS100A-R1 |
| | | T002664 | 41 | hSMN2_ISS-N1A-L1 |
| | | T002664 | 42 | hSMN2_ISS-N1A-R1 |
| IDOL | | T002665 | 43 | hIDOL_Ex2A-L1 |
| | | T002665 | 44 | hIDOL_Ex2A-R1 |
| | | T002666 | 45 | hIDOL_Ex3A-L1 |
| | | T002666 | 46 | hIDOL_Ex3A-R1 |
| ANGPTL3 | Combined | T002667 | 47 | hANGPTL3_Ex1A-L1 |
| | hyperlipidemia/ | T002667 | 48 | hANGPTL3_Ex1A-R1 |
| | familial | T002668 | 49 | hANGPTL3_Ex2A-L1 |
| | mixed | T002668 | 50 | hANGPTL3_Ex2A-R1 |
| | hyperlipidemia | | | |
| PCSK9 | (Familial) | T002669 | 51 | hPCSK9_Ex3A-L1 |
| | hyper- | T002669 | 52 | hPCSK9_Ex3A-R1 |
| | cholesterolemia | T002670 | 53 | hPCSK9_Ex12A-L1 |
| | | T002670 | 54 | hPCSK9_Ex12A-R1 |
| | | T002672 | 55 | hPCSK9_Ex3_AN-L1 |
| | | T002672 | 56 | hPCSK9_Ex3A-R1 |
| | | T002673 | 57 | hPCSK9_Ex12A-L1 |
| | | T002673 | 58 | hPCSK9_Ex12A-R1 |

### DETAILED DESCRIPTION OF THE INVENTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, N.Y.: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

In a general aspect, the present invention relates to methods for encapsulating an endonuclease reagent, comprising the steps of:
a) Engineering a endonuclease reagent,
b) Complexing said endonuclease reagent with at least one biodegradable matrix comprising at least a core hydrophobic domain and a proximal polar domain,
c) Forming particles encapsulating said endonuclease reagent of 50 to 150 nm diameter range;

The particles are formed from by the association of macromolecular structures as shown in FIG. 1, which are detailed further on. These structures self-assemble due to their hydrophobic and hydrophilic domains upon rapid mixing by microfluidic mixing techniques, which permit millisecond mixing at the nanoliter scale with polydispersity indexes as low as, or lower than 0.02, as described by Song et al. (Microfluidic synthesis of nanomaterials (2008) Small 4:698-711). The particles may be formed by one or several types of those structures. Chimeric Proteins comprising a non-polar or transmembrane domain and displaying a hydrophilic external affinity domain may be mixed with the other structural matrix structures to have these proteins anchored outwards the particles.

The elementary structures that build up the capsules by microfluidic mixing are preferably “biodegradable matrix”, meaning that that they can be made of various materials that can be degraded or eliminated by action of the enzymes naturally present into the body, preferably into the human body.

By “diameter range” is meant that the diameter is not strictly uniform. It corresponds to a statistical measure (distribution of the diameter of a number of particles) centered on a major value. This value is generally comprised between 50 and 150 nm, and more generally preferably set between 50 and 100 nm, more preferably between 50 and 90 nm, and even more preferably between 60 and 80 nm.

By “endonuclease reagent” is meant a nucleic acid molecule that contributes to an endonuclease catalytic reaction in the target cell, itself or as a subunit of a complex, preferably leading to the cleavage of a nucleic acid sequence target. The “endonuclease reagents” of the invention are generally sequence-specific reagents, meaning that they can induce DNA cleavage in the cells at predetermined loci, referred to by extension as “gene targets”, by specific recognition of a nucleic acid “target sequence”. Said target sequence is usually selected to be rare or unique in the cell's genome, and more extensively in the human genome, as determined by using the available human genome databases and related common software.

“Rare-cutting endonucleases” are sequence-specific endonuclease reagents of choice, insofar as their recognition sequences generally range from 10 to 50 successive base pairs, preferably from 12 to 30 bp, and more preferably from 14 to 20 bp.

According to a preferred aspect of the invention, said endonuclease reagent is a nucleic acid encoding an “engineered” or “programmable” rare-cutting endonuclease, such as a homing endonuclease as described for instance by Arnould S., et al. (WO2004067736), a zing finger nuclease as described, for instance, by Urnov F., et al. (Highly efficient endogenous human gene correction using designed zinc-finger nucleases (2005) Nature 435:646-651), a TALE-Nuclease as described, for instance, by Mussolino et al. (A novel TALE nuclease scaffold enables high genome editing activity in combination with low toxicity (2011) Nucl. Acids Res. 39(21):9283-9293), or a MegaTAL nuclease as described, for instance by Boissel et al. (MegaTALs: a rare-cleaving nuclease architecture for therapeutic genome engineering (2013) Nucleic Acids Research 42 (4):2591-2601).

According to the invention, the endonuclease reagent is preferentially under RNA form to allow transient endonuclease activity of said reagent into the target cell and make the entire capsule biodegradable in-vivo. Even more preferably, the endonuclease reagent is under the form of a mRNA for the expression of the rare cutting endonuclease into the cells. The endonuclease under mRNA form is preferably synthetized with a cap to enhance its stability according to techniques well known in the art, as described, for instance, by Kore A. L., et al. (Locked nucleic acid (LNA)-modified dinucleotide mRNA cap analogue: synthesis, enzymatic incorporation, and utilization (2009) J Am Chem Soc. 131(18):6364-5).

Due to their higher specificity, TALE-nuclease have proven to be particularly appropriate for therapeutic applications, especially under heterodimeric forms—i.e. working by pairs with a “right” monomer (also referred to as “5′” or “forward”) and ‘left” monomer (also referred to as “3′” or “reverse”) as reported for instance by Mussolino et al. (TALEN® facilitate targeted genome editing in human cells with high specificity and low cytotoxicity (2014) Nucl. Acids Res. 42(10): 6762-6773). However, before the invention, it was difficult to deliver in-vivo pairs of TALE-nuclease, even using viral vectors, especially because TAL-nuclease are large proteins having long genetic sequences. Delivery of these reactive proteins in-vivo was therefore remaining a considerable challenge before the present invention. This was all the more challenging that the proteins are active and specific when they are simultaneously delivered into the cell nucleus. Otherwise, the activity may be lost or the reagents may become less specific increasing the risk of off-site mutations.

The inventors have more particularly sought how to efficiently target cccDNA (covalently closed circular DNA), an intracellular viral replication intermediate of some viruses, which forms persistence reservoir and key obstacle for a cure of viral disease.

The cccDNA of HBV is at the origin of some common forms of chronic hepatitis B. Upon infection, cccDNA is generated as a plasmid-like episome in the host cell nucleus from the protein-linked relaxed circular (RC) DNA genome in incoming virions. It has a fundamental role as template for all viral RNAs, and in the production of new virions in the liver cells (Nassal et al., HBV cccDNA: viral persistence reservoir and key obstacle for a cure of chronic hepatitis B. (2015) Gut 64 (12):1972-1984).

Beyond the difficulty to deliver specific TALE-nucleases into the nucleus of liver cells, it must be recalled that HBV comprises different genotypes, at least 24 subtypes, displaying genome variability, which are reported to respond to treatment in different ways (Palumbo E., Hepatitis B genotypes and response to antiviral therapy: a review. (2007). American Journal of Therapeutics 14 (3): 306-9). As detailed in Example 1 herein, the inventors have designed specific TALE-nucleases that are able to target both the cccDNA of the main HBV subtypes, namely at least type A, B and C. The sequences of the successful TALE-nucleases used by the inventors to target HBV are listed in Table 7.

The present application claims any of the polypeptide or polynucleotide sequences having at least 80% identity, preferably at least 90%, more preferably 95%, and even more preferably 99% identity with any sequences referred to in Table 7. The present application claims the polynucleotides encoding said sequences, especially under RNA form.

The present invention more broadly provides a method for delivering in-vivo two endonuclease reagents under RNA form, preferably under mRNA, form to be expressed simultaneously into a cell.

Accordingly, the invention has also for object a pair of heterodimeric TALE-nucleases, preferably those targeting the cccDNA of HBV such as those referred in Table 2, which are encapsulated according to the method described herein to target liver cells—i.e.: into a biodegradable delivery capsule for gene targeting of a cell in vivo, characterized in that a endonuclease reagent under RNA form is complexed with at least one polar domain, which is linked to biodegradable conjugate(s) of hydrophobic monomers to form spherical particles as previously described.

According to another embodiment, the endonuclease reagent is a RNA-guide to be used in conjunction with a RNA guided endonuclease, such as Cas9 or Cpf1, as per, inter alia, the teaching by Doudna, J., and Chapentier, E., (The new frontier of genome engineering with CRISPR-Cas9 (2014) Science 346 (6213):1077), which is incorporated herein by reference.

According to a preferred aspect the RNA guide is complexed with its associated RNA-guided endonuclease protein into the hydrophilic domain of the capsule. Alternatively the RNA-guided endonuclease protein can be retained within the hydrophilic core of the micelle structure (situation where the elementary structures have a second distal hydrophilic domain as illustrated in FIG. 1—structures A and B).

According to another embodiment, at least two different endonuclease reagents are encapsulated under RNA form into the particles, which means that, for instance, a RNA guide and mRNA encoding a RNA guided endonuclease can be both complexed with the distal hydrophilic domain of the matrix.

The present method provides that the core hydrophobic domain can be a biodegradable conjugate of hydrophobic monomers.

According to one aspect of the invention, said hydrophobic monomers conjugate comprise aminolipids, such as ionized cationic lipid 1,2-dilinoleyloxy-3-dimethylaminopropane (DLinDMA) as described elsewhere, for instance, by Hafez, I. M. et al. (On the mechanism whereby cationic lipids promote intracellular delivery of the polynucleic acids (2001) Gene Therapy 8:1188-1196). Said aminolipids can be advantageously mixed with PEG-lipids, since endogenous apolipoprotein E (Apo E) specifically targets these delivery systems to hepatocytes by Apo E-dependent, receptor mediated endocytosis. The core hydrophobic domain can also include structural lipids such as cholesterol and saturated phosphatidylcholine. Apo E mediated endocytosis is one of the mechanism by which capsules comprising hydrophobic domain with PEG-lipids more particularly target liver cells by mimicking intermediate-density lipoprotein (IDL).

According to an alternative aspect of the invention, said hydrophobic domain comprises a polymer, such as poly-N,N-di(C1-C6)alkyl-amino(C1-C6)alkyl-ethacrylate, poly-N,N-di(C1-C6)alkyl-amino(C1-C6)alkyl-methacrylate, or poly-N,N-di(C1-C6)alkyl-amino(C1-C6)alkyl-acrylate, or a combination thereof. Preferentially, said hydrophobic domain comprises monomers including at least (C2-C8)alkyl-ethacrylate, a (C2-C8)alkyl-methacrylate, or a (C2-C8)alkyl-acrylate, which can be mixed with carboxylic acid monomers and tertiary amino monomers.

According to the present invention, a proximal polar “targeting domain” can be covalently linked to the core hydrophobic domain to specifically target desired cell type or tissue. Such targeting domain can be linked through usual peptide linker, such as Gly-rich linkers (GSₙ linkers) according to standard procedures known in the art. By “targeting domain” is meant any molecule that may be inserted or linked to the matrix providing more affinity of the capsule to a cell type, more generally to a specific cell surface marker.

Said targeting domain are mostly ligand or binding domains that are reported to have some affinity with cell surface receptors or antigens. Binding domains can be fusion proteins comprising ScFvs from antibodies generated against a cell surface antigen.

According to a preferred embodiment of the invention, the targeting domain recognizes a cell surface antigen from a LDL or VLDL receptor, which are abundantly present at the surface of liver cells, allowing easier internalization of the delivery capsule. According to another embodiment, the targeting domain has affinity with heparan sulfate proteoglycans.

According to another aspect of the invention N-acetylgalactosamine ligand is used as a targeting domain as a ligand of for asialoglycoprotein receptors (ASGP-r). Galactoside-containing cluster ligands, in particular glycopeptides containing N-acetyl-D-galactosamine (GaINAc) have high affinity to ASGP-r, which are found in abundance in mammalian parenchymal liver cells. Such ligands may be conjugated with the core hydrophobic domain to improve the efficiency of delivery to diseased liver cells as previously described by Wu Y. T., et al. (A new N-acetylgalactosamine containing peptide as a targeting vehicle for mammalian hepatocytes via asialoglycoprotein receptor endocytosis (2004) Curr Drug Deliv. April; 1(2):119-27).

As per the method described above, the invention provides with biodegradable delivery capsule for performing gene targeting into a cell in-vivo. These delivery capsules, are, at least in part, characterized in that a RNA endonuclease reagent is complexed with at least one polar domain, which can be linked to biodegradable conjugate(s) of hydrophobic monomers, under the form of spherical particles of 50 to 100 nm diameter range. The structure of these delivery capsules allowed the inclusion of at least two RNA endonuclease reagents, such as TALE nucleases, which was surprising given the reduced size of the particles.

According to one aspect, said biodegradable matrix that is complexed with the RNA endonuclease reagents comprises at least two polar domains, in such a manner that the inner core particle is hydrophilic. The inner core particle may then encapsulate a further endonuclease reagent, in particular under polypeptide form, such as a RNA or DNA-guided endonuclease, for instance, a Cas9 or Cpf1 protein.

Various combinations of reagents can be included in the biodegradable delivery capsules of the present invention, as illustrated in FIGS. 1 to 3.

The present invention is also drawn to a medicament or pharmaceutical compositions permitting the safe injection in-vivo of the above biodegradable delivery systems in view of editing a target gene. The medicament or pharmaceutical compositions of the present invention ideally comprise a pharmaceutically acceptable medium, preferably a pharmaceutically injectable medium. By “pharmaceutically injectable medium” is meant a suitable pharmaceutical carrier, which are well known in the art, such as phosphate buffered saline solutions, water, emulsions, such as oil/water emulsions, various types of wetting agents, sterile solutions, etc. Injections according to the present invention can be intracerebral, intramuscular, inside spinal chord or subcutaneous.

Preparations for intravenous administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin.

The pharmaceutical compositions according to the present invention can have many different therapeutic indications. Examples of indications are those referred to in Table 1. Specifically, Table 1 lists particular target genes in connection with several diseases, which can be treated using the endonuclease delivery means according to the present invention. One particular aspect of this approach is the gene editing of these specific target genes in view of obtaining the treatment of their associated disease in vivo.

As a preferred embodiment is the therapeutic use of the endonuclease reagents of the present invention to target in-vivo the genetic sequences expressing microRNAs involved into drug resistance in cancer treatment, especially after prolonged cycles of chemotherapy. Proposed targets in this respect are sequence encoding miRNA genes, especially those from let7, miR-21, mir-26, mir-10, mir-34 and/or mir-122 families. Drug resistance is a major problem in the treatment of cancer patients. Resistance can develop after prolonged cycles of chemotherapy or can be present intrinsically in the patient. There is an emerging role of microRNAs (miRNAs) in resistance to cancer treatments. miRNAs are small non-coding RNAs that are evolutionarily conserved and also involved as regulators of gene expression through the silencing of mRNA targets. They are involved in many different cancer types and a plethora of mechanisms have been postulated for the roles that miRNAs play in the development of drug resistance. Hence, miRNA-based gene therapy may provide a novel approach for the future of cancer therapy. This review focuses on an overview of recent findings on the role of miRNAs in the resistance to chemotherapy in different tumors.

According to a preferred embodiment of the invention, the biodegradable endonuclease delivery capsules are used against infection agents' genomes, in particular those agents that presents a DNA intermediate into the liver. By “DNA intermediate into the liver” is meant that the infectious agent has, even temporarily, at least one intermediate stage of its replication taking place into a hepatic cell under a DNA form.

HBV is such as infectious agent that presents a DNA intermediate as per its cccDNA, which forms a reservoir for the virus lodged into hepatic cells. Chronic hepatitis is mostly due to this cccDNA remaining in hepatic cells.

### Anti-HBV Combination Therapies Involving Specific TALE-Nucleases

One aspect of the present invention is to provide with new endonuclease reagents, especially TALE-nucleases (TALEN presented in Table 1) under RNA form for encapsulation into delivery particles for in-vivo targeting of HBV into liver cells. Preferred target sequences and TALEN monomers are those identified by the inventors in Example 1, through the cloning of large pieces of cccDNA into the genome of HEK293 cells (FIG. 4).

The invention more particularly provides endonuclease reagents engineered to target the cccDNA of HBV in-vivo which binds one target sequence selected from SEQ ID NO: 59 to 78, especially specific TALE-nucleases.

Also, the present invention provide TALE-nuclease monomers, which polypeptide sequence have at least 80%, preferably 90%, more preferably 95%, even more preferably 99% identity with any of SEQ ID NO. 1 to 20. These TALE-nuclease monomers are useful for treating HBV related infections. Preferred TALE-nucleases are those displaying at least 80% identity with SEQ ID NO. 1, 2, 3, 4, 5, 6, 9, 10, 13, 14, 15, 16, 17 and 18 (TALEN 1212, 1213, 1214, 2559, 2561, 2562, and 2563 respectively displaying more than 40% activity as shown in Example 1).

The TALE-nucleases described herein are particularly useful in combination with at least one antiviral compound to potentiate its effect against viral infection. Said antiviral compound is expected to block the virus propagation at different levels: viral transcription, replication and expression, HBV entry into cells, capsid assembly, and patient's immune response. The combination of the endonuclease reagent with such compound results into dramatic drop of viral load and diminution of chronic infections.

According to an embodiment of the present invention said antiviral compound, which is combined with the endonuclease agent, prevents the entry of HBV into liver cells and is preferably selected from an Inhibitor of sodium taurocholate cotransporting polypeptide (NTCP) [Nakabori T. et al. (2016) Sodium taurocholate cotransporting polypeptide inhibition efficiently blocks hepatitis B virus spread in mice with a humanized liver. Scientific Reports 6, Article number: 27782]. Examples of inhibitor of NTCP are Myrcludex B, which is a lipopeptide derived from HBV preS1 domain sequence, blocks de novo HBV infection [Urban et al. (2014) Gastroenterology. 147(1):48-64], the antagonist RO 41-5253 of Retinoic acid receptor, which is known to regulate the promoter activity of hNTCP, [Tsukuda et al. (2015) J Biol Chem. 290(9):5673-84] and IL-6 that is known to down regulate NTCP expression [Bouezzedine et al. (2015) Virology. 481:34-42].

According to an embodiment of the present invention said antiviral compound, which is combined with the endonuclease agent, is a cccDNA inhibitor, which blocks cccDNA formation. Examples of cccDNA inhibitors are disubstituted sulfonamide (DSS) compounds: CCC-0975 and CCC-0346 [Cai et al. (2012) Identification of Disubstituted Sulfonamide Compounds as Specific Inhibitors of Hepatitis B Virus Covalently Closed Circular DNA Formation Antimicrob. Agents Chemother. 56(8):4277-4288].

According to an embodiment of the present invention, said antiviral compound, which is combined with the endonuclease reagent, Inhibits viral expression or replication. There are several types of such compound. Preferred type consists of RNA interfering molecules, such as ARC-520 (developed by Arrowhead Research Corporation) [Gish et al. (2015) Synthetic RNAi triggers and their use in chronic hepatitis B therapies with curative intent. Antiviral Res. 121:97-108], ARO-HBV, which silences all HBV gene products and intervenes upstream of the reverse transcription process, ARB-001467 (developed by Arbutus NCT02631096) and GSK3228836 or GSK3389404 (also known as IONIS HBVRx or IONIS HBV LRX respectively) that are currently evaluated in clinical trials (https://clinicaltrials.gov: NCT02981602, NCT03020745 or NCT03020745, NCT02647281 respectively).

Other types of compounds that can be used to interfere with viral replication or expression in combination with the endonuclease reagent of the invention are Helioxanthin (HE-145) or analogues thereof, suppressing HBV gene expression and replication by selectively modulating the host transcriptional machinery [see in review Kang et al. (2015) Anti-HBV Drugs: Progress, Unmet Needs, and New Hope. Viruses. 7(9):4960-4977]

Ethanol extract from Ampelopsis sinica root (coming from Chinese medicine), which has also inhibiting activities against several HBV promoters and p53 associated signaling pathway (see in review Kang et al. 2015, above) can also be advantageously combined with the endonuclease reagents as per the therapeutic methods of the present invention.

Class I, II and III histone deacetylase inhibitors: p300 and P300/CBP associated factor histone acetyltransferases inhibitors, hSirt1 activators; JMJD3 histone demethylase inhibitors (such as GSK J4).[see review Phyo et al., (2015) Search for a cure for chronic hepatitis B infection: How close are we? World J Hepatol. 7(9):1272-1281] can also be advantageously combined with the endonuclease reagents of the present invention.

Another type of compound that interfere with transcription and/or replication to be advantageously used in combination with the endonuclease reagents of the present invention are regulators of La protein La protein is a phosphoprotein involved in HBV replication. An example of such regulator, which is preferred in combination with the endonuclease reagent(s), is HBSC11 (Tang et al. (2012) A Novel Inhibitor of Human La Protein with Anti-HBV Activity Discovered by Structure-Based Virtual Screening and In Vitro Evaluation. PLoS One.; 7(4):e36363.Epub 2012 Apr. 27.)

According to an embodiment of the present invention said antiviral compound, which is combined with the endonuclease reagent, prevents viral capsid assembly, and is preferably CpAMs (Capsid Allosteric Modulators). Examples of capsid allosteric modulators are ABI H0731, a viral core protein modulator (Assembly Biosciences) [Venkatakrishnan et al. (2016) Hepatitis B Virus Capsids Have Diverse Structural Responses to Small-Molecule Ligands Bound to the Heteroaryldihydropyrimidine Pocket. J. Virol. 90(8):3994-4004], JNJ 56136379 (or JNJ379) (Janssen Sciences Ireland UC) [Lam et al. (2017) HBV Capsid Assembly Modulators, but not Nucleoside Analogs, Inhibit the Production of Extracellular Pregenomic RNA and Spliced RNA Variants. Chemotherapy. Antimicrob. Agents Chemother. doi:10.1128/AAC.00680-17]. NVR 3 778 or NVR1221 (Novira Therapeutics) [Klumpp et al. (2015) High-resolution crystal structure of a hepatitis B virus replication inhibitor bound to the viral core protein. PNAS. 112(49):15196-2011, Morphothiadine (GLS4) (HEC Pharm, China, Ren et al. (2017) Discovery and Pre-Clinical Characterization of Third-Generation 4-H Heteroaryldihydropyrimidine (HAP) Analogues as Hepatitis B Virus (HBV) Capsid Inhibitors Bioorg Med Chem. 25(3):1042-1056], and AB-423 (Arbutus) [Cole, A. G. (2016) Modulators of HBV capsid assembly as an approach to treating hepatitis B virus infection Curr Opin Pharmacol. 30:131-137].

According to an embodiment of the present invention said antiviral compound, which is combined with the endonuclease reagent, prevents reverse transcription and is preferably a nucleoside or nucleotide analogue. Examples of nucleoside analogs are Lamivudine, Telbivudine, Entecavir, Adefovir, Tenofovir, in particular Tenofovir alafenamide (TAF or brand name Vemlidy®, registered for Hepatitis B by Gilead) [Agarwal et al. (2015) Management of chronic hepatitis B before and after liver transplantation. J Hepatol. 62(3):533-40], Besifovir (LB80380), MIV-210: a 2,3-dideoxy-3-fluorogguanosine (FLG), a fluorinated guanosine analogue, MCC-478 or Alamifovir (adefovir derivative).

According to an embodiment of the present invention said antiviral compound, which is combined with the endonuclease reagent, prevents viral HBsAg release, and preferably is a nucleic acid-based polymers that have sequence independent properties of phosphorothioated oligonucleotides to generate novel amphipathic polymers which have a very broad spectrum antiviral activity against enveloped viruses, such as REP2139 (Replicor) [see in Elazar et al. (2017) Emerging concepts for the treatment of hepatitis delta. Curr Opin Virol. 24:55-59]. Other examples of compounds interfering with HBsAg release are GC1102 (Green Cross Corporation) [https://clinicaltrials.gov: NCT02569372] and HBsAg shRNA or siRNA [see in Cheng et al., (2005) Dynamics of in vivo hepatitis D virus infection. Journal of Theoretical Biology. 398:9-19; and in Moore et al. (2005) Stable inhibition of hepatitis B virus proteins by small interfering RNA expressed from viral vectors. J. Gene Med. 7:918-925].

According to an embodiment of the present invention said antiviral compound, which is combined with the endonuclease agent, is an Immunoregulator agent, such as Thymosin-al (Thymalfasin). Thymosin-al promotes differentiation of T cells to a mature stage and thereby enhances the response to antigens and other excitants. The action of boosting the host immune system helps mounting a defense against chronic HBV infection. It can also be combined with IFN to treat chronic hepatitis B, in addition to the nuclease reagent as per the present invention. Vesatolimod (GS-9620) is another example of such immunomodulatory compound (Gilead). GS-9620 is an agonist of Toll Like Receptor (TLR) 7. TLRs are down regulated by HBV to reduce immune response to infection (IFNs, pro-inflammatory cytokines, chemokines production) [Isogawa et al. (2005) Toll-Like Receptor Signaling Inhibits Hepatitis B Virus Replication In Vivo. J. Virol. 79(11):7269-72][Lanford et al. (2013) GS-9620, an Oral Agonist of Toll-Like Receptor-7, Induces Prolonged Suppression of Hepatitis B Virus in Chronically Infected Chimpanzees. Gastroenterology. 144(7):1508-17].

According to an embodiment of the present invention said antiviral compound, which is combined with the endonuclease reagent, is vaccines mounting patient's immunity against HBV. Examples of such vaccines are GS-4774, alone (Gaggar et al. (2014) Safety, tolerability and immunogenicity of GS-4774, a hepatitis B virus-specific therapeutic vaccine, in healthy subjects: A randomized study. Vaccine. 32(39):4925-31]. or in combination with Tenofovir (TDF) (Gilead) [https://clinicaltrials.gov: NCT02174276.], ABX-203 (Abivax S.A.) [see in Elvidge, S. (2015) Blockbuster expectations for hepatitis B therapeutic vaccine. Nat. Biotechnol. 33(8):789], TG-1050 (Transgene) [Martin et al. (2015) TG1050, an immunotherapeutic to treat chronic hepatitis B, induces robust T cells and exerts an antiviral effect in HBV-persistent mice. Gut. 64(12):1961-71], INO-1800: (Inovio Pharmaceuticals) [Lucifora et al. (2014) Specific and Nonhepatotoxic Degradation of Nuclear Hepatitis B Virus cccDNA. Science. 343(6176):1221-8] and FP-02.2 (also known as HepTCell Altimmune, Inc.) [https://clinicaltrials.gov:NCT02496897].

According to an embodiment of the invention, the endonuclease reagent is combined with an agent that provides immuno-stimulation. Examples of such agents are SB-9200 (Spring Bank Pharmaceuticals, Inc.). SB 9200 is thought to activate the viral sensor proteins, retinoic acid-inducible gene 1 (RIG-I) and nucleotide-binding oligomerization domain-containing protein 2 (NOD2) resulting in interferon (IFN) mediated antiviral immune responses in virus-infected cells [Korolowicz et al. (2016) Antiviral Efficacy and Host Innate Immunity Associated with SB 9200 Treatment in the Woodchuck Model of Chronic Hepatitis B. PLoS ONE. 11(8): e0161313], AIC649, which has been shown to directly address the antigen presenting cell arm of the host immune defense leading to a regulated cytokine release and activation of T cell responses [Paulsen et al. (2015) AIC649 Induces a Bi-Phasic Treatment Response in the Woodchuck Model of Chronic Hepatitis B, PLoS ONE 10(12):e0144383] [Ebert et al. (2015) Eliminating hepatitis B by antagonizing cellular inhibitors of apoptosis. PNAS. 112(18):5803-8], cytokines, such as IL-21 (Publicover et al. (2011) IL-21 is pivotal in determining age-dependent effectiveness of immune responses in a mouse model of human hepatitis B. J. Clin. Invest. 121(3):1154-62], or IL-7, such as CYT107 a recombinant human IL-7 tested in clinical trial [https://clinicaltrials.gov:NCT01027065], or antibodies antagonist of Immune checkpoint, such as Nivolumab and Pembrolizumab.

A further aspect of the present invention is a method for delivering an endonuclease reagent into a cell in vivo, comprising the step of:
Producing a biodegradable delivery capsule as previously described; and
Injecting, parenterally or enterally, preferably intravenously said capsule into the blood circulation of a patient;

More broadly, the present invention can be regarded as a method for treating a HBV infection, comprising the steps of introducing into the blood stream of an animal a biodegradable delivery capsule comprising an endonuclease reagent, such as one described before, together with another antiviral compound selected from:
Inhibitor of sodium taurocholate cotransporting polypeptide (NTCP), such as Myrcludex;
cccDNA inhibitor, such as disubstituted sulfonamide (DSS) compounds, antibodies inducing Lymphotoxin beta receptor activation or LTβR agonists;
RNAi or compounds aiming at reducing HBV genes expression, in particular Helioxanthin or Ethanol extract from Ampelopsis sinica root;
La protein inhibitor, such as HBSC11;
Capsid allosteric modulators, such as ABI H0731, JNJ 56136379 (or JNJ379), Morphothiadine (GLS4), NVR 3 778 or NVR1221;
Reverse transcriptase inhibitors, in particular nucleoside analogs, such as Lamivudine, Telbivudine, Entecavir, Adefovir, Tenofovir, Besifovir, MIV-210, MCC-478 or Alamifovir;
Inhibition of HBsAg release, such as REP2139 and GC1102;
Immunoregulators, such as Thymosin-al;
Vesatolimod (GS-9620);
Vaccines, such as GS-4774, ABX-203, TG-1050, INO-1800; FP-02.2;
Immune stimulators, such as SB-9200, AIC649, Cellular inhibitor of apoptosis proteins (cIAPs) Cytokines: IL-21 and/or recombinant human IL-7 and antibodies antagonist of Immune checkpoint (Nivolumab and Pembrolizumab), or any equivalent antiviral agents. By equivalent agent is meant another molecule that is reported in the art to share the same mode of action leading to a similar biological effect, preferably by acting on the same receptor, intermediate or biological pathway.

More specifically, the invention is drawn to compositions for use in the treatment of HBV infection, comprising the following combinations:
A HBV specific endonuclease reagent and Inhibitor of sodium taurocholate cotransporting polypeptide (NTCP), such as Myrcludex;
A HBV specific endonuclease reagent and disubstituted sulfonamide (DSS) compounds;
A HBV specific endonuclease reagent and at least one antibody inducing Lymphotoxin beta receptor activation
A HBV specific endonuclease reagent and at least one LTβR agonist;
A HBV specific endonuclease reagent and Helioxanthin;
A HBV specific endonuclease reagent and an Ethanol extract from Ampelopsis sinica root;
A HBV specific endonuclease reagent and at least one La protein inhibitor, such as HBSC11;
A HBV specific endonuclease reagent and at least one capsid allosteric modulator, such as ABI H0731;
A HBV specific endonuclease reagent and at least one capsid allosteric modulator, such as JNJ 56136379 (or JNJ379);
A HBV specific endonuclease reagent and at least one capsid allosteric modulator, such as Morphothiadine (GLS4);
A HBV specific endonuclease reagent and at least one capsid allosteric modulator, such as NVR 3 778;
A HBV specific endonuclease reagent and at least one capsid allosteric modulator, such as NVR1221;
A HBV specific endonuclease reagent and at least one reverse transcriptase inhibitor, such as Lamivudine;
A HBV specific endonuclease reagent and at least one reverse transcriptase inhibitor, such as Telbivudine;
A HBV specific endonuclease reagent and at least one reverse transcriptase inhibitor, such as Entecavir;
A HBV specific endonuclease reagent and at least one reverse transcriptase inhibitor, such as Adefovir;
A HBV specific endonuclease reagent and at least one reverse transcriptase inhibitor, such as Tenofovir;
A HBV specific endonuclease reagent and at least one reverse transcriptase inhibitor, such as Besifovir;
A HBV specific endonuclease reagent and at least one reverse transcriptase inhibitor, such as MIV-210,
A HBV specific endonuclease reagent and at least one reverse transcriptase inhibitor, such as MCC-478;
A HBV specific endonuclease reagent and at least one reverse transcriptase inhibitor, such as Alamifovir;
A HBV specific endonuclease reagent and at least one inhibitor of HBsAg release, such as REP2139;
A HBV specific endonuclease reagent and at least one inhibitor of HBsAg release, such as GC1102;
A HBV specific endonuclease reagent and at least one immunoregulator, such as Thymosin-α1;
A HBV specific endonuclease reagent and Vesatolimod (GS-9620);
A HBV specific endonuclease reagent and at least one vaccine, such as GS-4774;
A HBV specific endonuclease reagent and at least one vaccine, such as ABX-203;
A HBV specific endonuclease reagent and at least one vaccine, such as TG-1050;
A HBV specific endonuclease reagent and at least one vaccine, such as INO-1800;
A HBV specific endonuclease reagent and at least one vaccine, such as FP-02.2;
A HBV specific endonuclease reagent and at least one immune stimulator, such as SB-9200;
A HBV specific endonuclease reagent and at least one immune stimulator, such as AIC649;
A HBV specific endonuclease reagent and at least one cellular inhibitor of apoptosis proteins (cIAPs);
A HBV specific endonuclease reagent and at least one cytokine, such as IL-21;
A HBV specific endonuclease reagent and at least one cytokine, such as IL-7;
A HBV specific endonuclease reagent and at least one antagonist of Immune checkpoint, such as Nivolumab;
A HBV specific endonuclease reagent and at least one antagonist of immune checkpoint, such as Pembrolizumab;

Said antiviral compounds or agents can be included in same of different biodegradable delivery capsules, and be released simultaneously or one after the other(s). They can also not be included in the capsules, while being simultaneously administered in the patient, preferably as part of the same injection.

According to such embodiment, the invention encompasses therapeutic compositions comprising antiviral compounds, such as those listed above, and delivery capsules, such as described before, containing an HBV specific endonuclease reagent, preferably a TALE-nuclease described herein. According to a preferred embodiment, delivery capsules comprise a composition including both the endonuclease reagent and said another antiviral compound.

According to a preferred embodiment the endonuclease reagent is present in the therapeutic compositions of the present invention under RNA form.

With respect to antiviral treatments, the endonuclease reagents of the present invention can be advantageously used in combination with further antiviral molecules, which preferably do not target cccDNA, in particular those selected from: lamivudine (Epivir), entecavir (Baraclude), adefovir (Hepsera), tenofovir (Viread), Telbivudine (Tyzeka, Sebivo), Pegylated Interferon (Pegasys) or Interferon Alpha (Intron A).

According to a further embodiment of the invention, the endonuclease reagent referred to before, such as preferably a TALE-nuclease targeting a HBV genome sequence present in cccDNA, is secreted or combined with engineered T-cells.

T-cells from donors or derived from stem cells may be engineered to be made less alloreactive by reducing or inactivating TCR expression as previously described by the applicant in WO2013176915.

Engineered T-cells can also express at their surface chimeric antigen receptors or recombinant TCR to specifically target infected liver cells, such as described by Krebs K. et al. [T-Cells Expressing a Chimeric Antigen Receptor That Binds Hepatitis B Virus Envelope Proteins Control Virus Replication in Mice (2013) Gastroenterology. 145:456-465].

The present invention encompasses therapeutic compositions comprising an endonuclease reagent directed against a sequence comprised into a HBV genome, preferably under encapsulated form, and at least one T-cell or a population of T-cells expressing receptor(s) that selectively binds HBV infected cells.

### Other Definitions

Amino acid residues in a polypeptide sequence are designated herein according to the one-letter code, in which, for example, Q means Gln or Glutamine residue, R means Arg or Arginine residue and D means Asp or Aspartic acid residue.
Amino acid substitution means the replacement of one amino acid residue with another, for instance the replacement of an Arginine residue with a Glutamine residue in a peptide sequence is an amino acid substitution.
Nucleotides are designated as follows: one-letter code is used for designating the base of a nucleoside: a is adenine, t is thymine, c is cytosine, and g is guanine. For the degenerated nucleotides, r represents g or a (purine nucleotides), k represents g or t, s represents g or c, w represents a or t, m represents a or c, y represents t or c (pyrimidine nucleotides), d represents g, a or t, v represents g, a or c, b represents g, t or c, h represents a, t or c, and n represents g, a, t or c.
“As used herein, “nucleic acid” or “polynucleotides” refers to nucleotides and/or polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Nucleic acids can be either single stranded or double stranded.
The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific polynucleotide sequences, further referred to as “target sequences” or “target sites”. Endonucleases can be classified as rare-cutting endonucleases when having typically a polynucleotide recognition site greater than 10 base pairs (bp) in length, more preferably of 14-55 bp. Rare-cutting endonucleases significantly increase homologous recombination by inducing DNA double-strand breaks (DSBs) at a defined locus thereby allowing gene repair or gene insertion therapies (Pingoud, A. and G. H. Silva (2007). Precision genome surgery. Nat. Biotechnol. 25(7): 743-4).
by “DNA target”, “DNA target sequence”, “target DNA sequence”, “nucleic acid target sequence”, “target sequence”, or “processing site” is intended a polynucleotide sequence that can be targeted and processed by a rare-cutting endonuclease according to the present invention. These terms refer to a specific DNA location, preferably a genomic location in a cell, but also a portion of genetic material that can exist independently to the main body of genetic material such as plasmids, episomes, virus, transposons or in organelles such as mitochondria as non-limiting example. As non-limiting examples of RNA guided target sequences, are those genome sequences that can hybridize the guide RNA which directs the RNA guided endonuclease to a desired locus.
By “delivery capsule” is intended any delivery mean which can be used in the present invention to put into cell contact (i.e “contacting”) or deliver inside cells or subcellular compartments (i.e “introducing”) the endonuclease reagents of the present invention. It includes, but is not limited to liposomal delivery vectors, viral delivery vectors, drug delivery vectors, chemical carriers, polymeric carriers, lipoplexes, polyplexes, dendrimers, microbubbles (ultrasound contrast agents), nanoparticles or emulsions.
by “mutation” is intended the substitution, deletion, insertion of up to one, two, three, four, five, six, seven, eight, nine, ten, eleven, twelve, thirteen, fourteen, fifteen, twenty, twenty five, thirty, forty, fifty, or more nucleotides/amino acids in a polynucleotide (cDNA, gene) or a polypeptide sequence. The mutation can affect the coding sequence of a gene or its regulatory sequence. It may also affect the structure of the genomic sequence or the structure/stability of the encoded mRNA.
by “variant” is intended a catalytically active mutant of an endonuclease reagent according to the present invention.
As used herein, the term “locus” is the specific physical location of a DNA sequence (e.g. of a gene) into a genome. The term “locus” can refer to the specific physical location of a rare-cutting endonuclease target sequence on a chromosome or on an infection agent's genome sequence. Such a locus can comprise a target sequence that is recognized and/or cleaved by a sequence-specific endonuclease according to the invention. It is understood that the locus of interest of the present invention can not only qualify a nucleic acid sequence that exists in the main body of genetic material (i.e. in a chromosome) of a cell but also a portion of genetic material that can exist independently to said main body of genetic material such as plasmids, episomes, virus, transposons or in organelles such as mitochondria as non-limiting examples.
The term “cleavage” refers to the breakage of the covalent backbone of a polynucleotide. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. Double stranded DNA, RNA, or DNA/RNA hybrid cleavage can result in the production of either blunt ends or staggered ends.
By “fusion protein” is intended the result of a well-known process in the art consisting in the joining of two or more genes which originally encode for separate proteins or part of them, the translation of said “fusion gene” resulting in a single polypeptide with functional properties derived from each of the original proteins.
“identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated.
The term “subject” or “patient” as used herein includes all members of the animal kingdom including non-human primates and humans.
The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

In summary, the present invention pertains to specific endonuclease reagents, such as the TALE-nucleases engineered to target the cccDNA of HBV in-vivo which binds one target sequence selected from SEQ ID NO: 1 to 20, which are preferably combined with antiviral compounds for the treatment of HBV in liver cells. The TALE-nucleases are preferably encapsulated according to one of the following embodiments:

1) A method for encapsulating an endonuclease reagent, comprising the steps of:
a) Engineering a endonuclease reagent under RNA form;
b) Complexing said endonuclease reagent with at least one biodegradable matrix comprising at least a core hydrophobic domain and a proximal polar domain to favor interactions with water molecules;
c) Forming particles encapsulating said endonuclease reagent of 50 to 100 nm diameter range.

2) A method according to embodiment 1, wherein said endonuclease reagent is a sequence-specific endonuclease reagent.

3) A method according to embodiment 1, wherein said endonuclease reagent is a rare-cutting endonuclease, such as a homing endonuclease, a zing finger nuclease, a TALE-Nuclease or a MegaTAL-endonuclease.

4) A method according to embodiment 3, wherein said rare-cutting endonuclease is a TALE-nuclease.

5) A method according to embodiment 1, wherein said RNA encodes an endonuclease reagent, which is a RNA-guided endonuclease.

6) A method according to embodiment 5, wherein said RNA-guided endonuclease is cas9 or Cpf1.

7) A method according to embodiment 1, wherein said RNA is a RNA-guide.

8) A method according to embodiment 7, wherein said RNA guide is complexed with a RNA-guided endonuclease protein.

9) A method according to any one of embodiments 1 to 8, wherein at least two different endonuclease reagents are encapsulated under RNA form into the particles.

10) A method according to embodiment 9, wherein said different endonuclease reagents are at least a RNA encoding a RNA-guided endonuclease and a guide RNA.

11) A method according to embodiment 1, wherein said core hydrophobic domain is a biodegradable conjugate of hydrophobic monomers.

12) A method according to embodiment 1, wherein said core hydrophobic and proximal polar domains are covalently linked.

13) A method according to embodiment 1, wherein said core hydrophobic and proximal polar domains are linked by peptide linkers.

14) A method according to embodiment 11, wherein said hydrophobic monomers conjugate are of aminolipids, such as ionized cationic lipid 1,2-dilinoleyloxy-3-dimethylaminopropane (DLinDMA).

15) A method according to embodiment 14, wherein said aminolipids are mixed with PEG-lipids to form said polar domain.

16) A method according to any one of embodiments 14 and 15, wherein said aminolipids allows binding of ApoE in-vivo, said ApoE facilitating Apo E mediated endocytosis.

17) A method according to any one of embodiments 1 to 16, wherein said endonuclease reagent endocytosis is mediated via a receptor of the LDL or VLDL receptor family.

18) A method according to any one of embodiments 1 to 17, wherein said at least one polar domain is poly-N,N-di(C1-C6)alkyl-amino(C1-C6)alkyl-ethacrylate, poly-N,N-di(C1-C6)alkyl-amino(C1-C6)alkyl-methacrylate, or poly-N,N-di(C1-C6)alkyl-amino(C1-C6)alkyl-acrylate, or a combination thereof.

19) A method according to embodiment 18, wherein said hydrophobic monomers include at least (C2-C8)alkyl-ethacrylate, a (C2-C8)alkyl-methacrylate, or a (C2-C8)alkyl-acrylate.

20) A method according to any one of embodiments 1 to 19, wherein said hydrophobic monomers conjugate are mixed with carboxylic acid monomers and tertiary amino monomers.

21) A method according to any one of embodiments 1 to 20, wherein said at least one polar domain is linked to a targeting domain.

22) A method according to embodiment 21, wherein said targeting domain comprises ScFv of an antibody targeting a cell surface antigen.

23) A method according to embodiment 22, wherein said cell surface antigen is a protein is selected from a LDL, VLDL receptors or cell surface heparin sulfate proteoglycans.

24) A method according to any one of embodiments 1 to 23, wherein said at least one polar domain is linked to a N-acetylgalactosamine ligand.

25) A method according to any one of embodiments 21, 22 and 24, wherein said targeting domain is a ligand of asiaglycoprotein.

26) A method according to embodiment 1, wherein said particles encapsulating said endonuclease reagent are of 50 to 90 nm diameter range.

27) A biodegradable delivery capsule obtainable by the method according to any one of embodiments 1 to 26.

28) A biodegradable delivery capsule for gene targeting of a cell in-vivo, characterized in that a RNA endonuclease reagent is complexed with at least one polar domain, which is linked to biodegradable conjugate(s) of hydrophobic monomers to form spherical particles of 50 to 100 nm diameter range.

29) A biodegradable delivery capsule according to embodiments 27 or 28, wherein at least two RNA endonuclease reagents are included into said spherical particles.

30) A biodegradable delivery capsule according to any one of embodiments 27 to 29, wherein said biodegradable matrix comprises at least two polar domains, such that the inner core particle is hydrophilic.

31) A biodegradable delivery capsule according to embodiment 30, wherein said hydrophilic inner core particle encapsulates a further endonuclease reagent.

32) A biodegradable delivery capsule according to embodiment 31, wherein said further endonuclease reagent comprises a polypeptide.

33) A biodegradable delivery capsule according to embodiment 32, wherein said polypeptide encodes a RNA or DNA guided endonuclease.

34) A pharmaceutical composition comprising a biodegradable delivery system according to any one of embodiments 27 to 33 with a pharmaceutically acceptable medium, preferably pharmaceutically injectable medium.

35) A pharmaceutical composition according to embodiment 34, for use as a medicament.

36) A pharmaceutical composition according to embodiment 34, for use in the treatment of a liver disease.

37) A pharmaceutical composition according to any one of embodiments 34 to 36, for use in the treatment of an infectious disease.

38) A pharmaceutical composition according to embodiment 36 or 37, wherein said disease is a viral disease such as hepatitis.

39) A pharmaceutical composition according to embodiment 37 or 38, wherein said infectious disease is due to an infectious agent that presents a DNA intermediate into the liver.

40) A pharmaceutical composition according to embodiment 39, wherein said infectious agent is a Hepadnavirus, such as HBV.

41) A pharmaceutical composition according to embodiment 34, for use in the treatment of malignant cells.

42) A method for delivering an endonuclease reagent to a cell in vivo, comprising the step of:
Producing a biodegradable delivery capsule by a method according to any one of embodiments 1 to 26;
Injecting intravenously said capsule into the blood circulation of a patient;

43) A method for gene editing a target gene into a cell in-vivo, comprising the steps of introducing a biodegradable delivery capsule according to any one of embodiments 27 to 33 into the blood stream of an animal.

44) A method for gene editing according to embodiment 43, wherein the target gene is one from the cccDNA of HBV.

Having generally described this invention, a further understanding can be obtained by reference to certain specific examples, which are provided herein for purposes of illustration only, and are not intended to be limiting unless otherwise specified.

### Example 1

Design of TAL-Nucleases Targeting HBV Subtypes cccDNA

Hepatitis B virus (HBV) is a member of the Hepadnaviridae family of viruses. Infection with HBV can lead to cirrhosis and hepatocellular carcinoma. The genome of HBV is made of circular DNA, which is not fully double-stranded. One end of the full length strand is linked to the viral DNA polymerase. The genome is ˜3000 nucleotides long (for the full-length strand) and ˜2000 nucleotides long (for the short length-strand). The partially double-stranded DNA is rendered fully double-stranded by completion of the (+) sense strand and removal of a protein molecule from the (−) sense strand and a short sequence of RNA from the (+) sense strand. Non-coding bases are removed from the ends of the (−) sense strand and the ends are rejoined. There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The function of the protein coded for by gene X is not fully understood but it is associated with the development of liver cancer. It stimulates genes that promote cell growth and inactivates growth regulating molecules.

The life cycle of hepatitis B virus is complex. Hepatitis B is one of a few known pararetroviruses: non-retroviruses that still use reverse transcription in their replication process. The virus gains entry into the cell by binding to NTCP on the surface and being endocytosed. Because the virus multiplies via RNA made by a host enzyme, the viral genomic DNA has to be transferred to the cell nucleus by cellular chaperones. The partially double stranded viral DNA is then made fully double stranded by viral polymerase and transformed into covalently closed circular DNA (cccDNA). This cccDNA serves as a template for transcription of four viral mRNAs by host RNA polymerase. The largest mRNA, (which is longer than the viral genome), is used to make the new copies of the genome and to make the capsid core protein and the viral DNA polymerase. These four viral transcripts undergo additional processing and go on to form progeny virions that are released from the cell or returned to the nucleus and re-cycled to produce more copies. The long mRNA is then transported back to the cytoplasm where the virion P protein (the DNA polymerase) synthesizes DNA via its reverse transcriptase activity.

The goal of the project was to generate TAL-nucleases able to cut HBV cccDNA and decrease its level in cell culture and in vivo. It should also cut the Relaxed Circular DNA (RC DNA), therefore acting of two different pools of virus. It could also cut integrated HBV partial genomes that are most often found in hepatocarcinomas.

There are no known inhibitors of the cccDNA. Some inhibitors are claimed to inhibit the formation of the cccDNA but none are targeting the pool of long lasting episomal reservoir of HBV in patients. The destruction of this pool could be a tool toward the cure of HBV.

HBV genome is difficult to express in cells line due to the complexity of its replication process and its toxicity. Also cells that produce HBV are difficult to transfect. We therefore decide to generate a stable cell line in 293 cells containing reference HBV genome (ayw) integrated at a known locus using a cGPS approach as described in WO2010046786, so that endonuclease reagent activity could be monitored in an easy and non-infectious manner.

HBV HEK 293 cells were very useful to select efficient TALE-nucleases targeting HBV. Cells M1-1000, M801-1800 and M1601-2006 were used to test TALE-nucleases: TALEN T002559-2564 (set 1) and TALEN T001212-1215 (set2). All of them were able to cut their target, with different level of efficiency. We were able to generate at least one very efficient TALEN per gene.

PCR on Transfected cGPS

The activity of the above TALENs was measured by transfecting plasmids in the above HBV 293 cGPS cells. 1.5 millions of each kind of HBV cGPS HEK 293 cells were plated in T25 the evening before transfection. 2 μg of each set of appropriate of plasmids (so 4 μg per TALEN, see table 1 and table 2) were transfected using Mirus TranslT-293 transfection reagent for 3 to 4 days. A control GFP was transfected in parallel (4 μg) for all cell lines and the efficiency of the transfection was monitored with a fluorescent microscope and estimated at ˜95%. Genomic DNA was then extracted using the Zymo Research quick-gDNA miniprep kit and PCR.

T7 Endonuclease Assay on HBV cGPS Cells Tranfected with TALENs

PCR products were annealed slowly, treated by T7 Endonuclease, run on a 10% polyacrylamide gel and stained with Sybergreen (T7 assay). One expects that the TALEN cut and NHEJ repair would result in the formation of a population of genomic DNA (and hence PCR product) that do not overlap at the surroundings of the TALEN cut site. After annealing those fragment would form a bulge that could be cleaved by the Endonuclease T7 and create fragments of expected sizes. Results are shown in FIGS. 5 and 6. After T7 assay, digested fragments of PCR appeared at the expected size for all the TALEN tested. The PCR bands and cut the products presented in FIGS. 5 and 6 were quantitated. Quantitation of digests with Biorad Image Lab program are presented in table 5. The efficiency of the cut was estimated by calculating the sum of the volume of the cut bands and dividing by the sum of the volume of total of the bands (cut and uncut). This approach does not take into account that the intensity of the bands is proportional to their size, and therefore this calculation is a rough estimate of the efficiency of the TALEN on the target.

All the TALEN tested had an estimated efficiency of more than 10%, while 6 were more than 40% active.

### Conclusion

HBV HEK 293 cells were very useful to select efficient TALENs targeting HBV. We were able to create at least one very efficient TALEN per gene. The activity of T002561 (which cuts S and P), T001212 (which cuts X and P), T002559 (which cuts C and P) was more than 40% based on T7 assay and are therefore eligible for being encapsulated for in-vivo targeting of liver cells.

TABLE 5
Quantitation of the PCR bands with
Biorad Lab Image program

| | | | | | % Cut |
|---|---|---|---|---|---|
| TALEN # | PCR bands | Volume | SUM | Volume | of Total |
| T002561 | PCR Uncut | 1624248 | Total | 5073480 | 68 |
| | PCR Cut 1 | 2180088 | Cut | 3449232 | |
| | PCR Cut 2 | 1269144 | | | |
| T002563 | PCR Uncut | 2315160 | Total | 3354624 | 31 |
| | PCR Cut 1 | 684432 | Cut | 1039464 | |
| | PCR Cut 2 | 355032 | | | |
| T002564 | PCR Uncut | 2654136 | Total | 3024216 | 12 |
| | PCR Cut 1 | 237312 | Cut | 370080 | |
| | PCR Cut 2 | 132768 | | | |
| T002559 | PCR Uncut | 2056032 | Total | 7750578 | 73 |
| | PCR Cut 1 | 3415896 | Cut | 5694546 | |
| | PCR Cut 2 | 2278650 | | | |
| T002560 | PCR Uncut | 2505030 | Total | 3014946 | 17 |
| | PCR Cut 1 | 308220 | Cut | 509916 | |
| | PCR Cut 2 | 201696 | | | |
| T002562 | PCR Uncut | 1507440 | Total | 5514894 | 73 |
| | PCR Cut 1 | 2345508 | Cut | 4007454 | |
| | PCR Cut 2 | 1661946 | | | |
| T001215 | PCR Uncut | 1675328 | Total | 2451712 | 32 |
| | PCR Cut 1 | 621312 | Cut | 776384 | |
| | PCR Cut 2 | 155072 | | | |
| T001212 | PCR Uncut | 1453760 | Total | 2613760 | 44 |
| | PCR Cut 1 | 690688 | Cut | 1160000 | |
| | PCR Cut 2 | 469312 | | | |
| T001213 | PCR Uncut | 939200 | Total | 2262848 | 58 |
| | PCR Cut 1 | 825408 | Cut | 1323648 | |
| | PCR Cut 2 | 498240 | | | |
| T001214 | PCR Uncut | 975552 | Total | 1789696 | 45 |
| | PCR Cut 1 | 632000 | Cut | 814144 | |
| | PCR Cut 2 | 182144 | | | |

### Example 2

In-Vivo Targeting of Factor VII Gene into the Genome of Liver Cells

The liver is a key organ for most metabolic pathways and therefore numerous metabolic inherited diseases have their origin in this organ. It is an attractive target for in vivo gene transfer studies due to the accessibility of the hepatocytes via the blood stream. The liver is the largest organ in the body and a highly vascularized organ. It is the only organ in the body to have two circulation systems, the systemic with the hepatic artery that brings oxygenated blood directly from the heart and the portal vein that brings nutrients from the gut and supplies 70% of the blood flow to the liver. In addition, it has a system of ducts that transports toxins out of the liver via bile into the small intestine, which is of importance for liver gene therapy applications since it allows hepatocytes to excrete bile salts, copper, bilirubin, etc, which cause liver diseases. Candidate diseases for liver gene therapy include primary liver diseases in which hepatocytes are injured and genetic defects altering a specific function of the hepatocyte but causing extrahepatic manifestations.

As a proof of principle, the liver has been chosen as a target organ in view of inactivating in-vivo mouse factor VII. Mouse Factor VII is a secreted protein secreted by liver cells, which can be easily quantitated in the blood. Inactivation of the gene in mouse liver is expected to lead to a decrease of the secreted protein into the circulating blood as described elsewhere (Akin, A. et al. (2009) Development of Lipidoid-siRNA Formulations. Molecular Therapy 17 5: 872-879.)

TAL-nucleases were engineered at the DNA level using the golden gate cloning assembly method described by Weber E., et al. (Assembly of Designer TAL Effectors by Golden Gate Cloning (2011) PLoS ONE 6(5): e19722) and cloned into a mammalian expression vector under the control of a pEF1alpha long promoter and tested in a human cell line by using a T7 experiment as described in Example 1.

Transcripts of mRNA encoding respectively forward (SEQ ID NO: 21 or 23) and reverse (SEQ ID NO: 20 or 22) of the most efficient TALEN monomer pairs were produced using standard procedure (Ambion kit, Thermo Fisher Scientific). The capped transcripts were complexed with ionized cationic lipid 1,2-dilinoleyloxy-3-dimethylaminopropane (DLinDMA) and PEG-lipids to generate 80 nm range diameter range nanoparticles for systemic delivery to the liver.

Controls were PBS (negative control) and siRNA against Factor VII (positive control from Ambion, #4457292 Thermofisher) as such siRNA have proven efficiency in previous studies.

The injection volume was 10 mL/kg (i.e for one mouse weighing 20 g, 200 μL of dosing solution is administered). Injection were at 2 mg/kg, of a 0.2 mg/ml solution, so that 200 μl were injected for a 20 g mouse, intravenously (IV, bolus) into the caudal vein of mice.

The treatment starts on Day 0 (D₀) using sixteen (18) healthy female CD-1 mice.

The treatment schedule is as follows:
The animals from group 1 received one single IV injection of Control Buffer (Q1D×1),
The animals from group 2 received one single IV injection of mRNA TALEN® complexed into capsules at 2 mgRNA/kg (Q1D×1),
The animals from group 3 received one ingle IV injection of Positive siRNA control (siRNA FVII) at 2 mg/kg (Q1D×1).

The treatment schedule is summarized in the table hereafter:

TABLE 6
Mice treatment schedule

| | Nb | | Dose | Adm. | Treatment |
|---|---|---|---|---|---|
| Group | animals | Treatment | (mg/kg/adm) | Route | schedule |
| 1 | 6 | Control Buffer | — | IV | Q1Dx1 |
| 2 | 6 | TALEN | 2 mg/kg | IV | Q1Dx1 |
| | | Complex | | | |
| 3 | 6 | Positive | 2 mg/kg | IV | Q1Dx1 |
| | | siRNA control | | | |

Blood was collected on D0 (just before IV treatment) and then on D3, D5, D10, D15, D20 and D25. All mice were terminated on D25. Approximately 150-200 μL of blood was collected at each time point, plasma was then flash-frozen in liquid nitrogen and stored at −80° C. Liver from each mouse was collected at the time of termination, flash-frozen in liquid nitrogen and stored at −80° C.

Factor VII activity in the collected plasma was measured using BIOPHEN FVII kit #221304.

Gene modifications were measured in livers by extracting genomic DNA on powdered frozen liver using ZR Genomic DNA™-Tissue MidiPrep from ZymoResearch #D3110, then performing a T7 assay and deep sequencing on the PCR product of the locus of interest.

Results show in FIG. 5, that a quickest effect is obtained with siRNA by D3 to D10. However by D15, siRNA starts to decline, whereas the inhibition induced by TALE-nucleases delivery remains stable up to D20. Together with the results of the deep sequencing (data not shown), it appears that the stable inhibition observed with the TALE-nucleases is due to mutations conferring permanent inactivation of a large number of factor VII gene copies in the liver cells.

TABLE 7
TALE-nucleases engineered to target the HBV genome, APOC3,
TTR, SMN2, IDOL, ANGPTL3 and PCSK9 genes, along with their
polypeptide sequences et target polynucleotide sequences

| | | | RVD | |
|---|---|---|---|---|
| TALEN | | | polynucleotide | |
| Number | Gene target | RVD Motif | target sequence | Polypeptide Sequence |
| T001212 | HBV1530_T01.L1 | HD-NG-NN-NG- | (SEQ ID NO: 59) | (SEQ ID NO: 1) |
| | | NN-HD-HD-NG- | CTGTGCCTTCTCATC | MGDPKKKRKVIDYPYDVPDYAIDIADLRTLGYSQQQQEK |
| | | NG-HD-NG-HD- | T# | IKPKVRSTVAQHHEALVGHGFTHAHIVALSQHPAALGTV |
| | | NI-NG-HD-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPQQV |
| | | | | VAIASNNGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASHDGGKQALET |
| | | | | VQRLLPVLCQAHGLTPEQVVAIASHDGGKQALETVQRLL |
| | | | | PVLCQAHGLTPQQVVAIASNGGGKQALETVQRLLPVLCQ |
| | | | | AHGLTPQQVVAIASNGGGKQALETVQRLLPVLCQAHGLT |
| | | | | PEQVVAIASHDGGKQALETVQRLLPVLCQAHGLTPQQVV |
| | | | | AIASNGGGKQALETVQRLLPVLCQAHGLTPEQVVAIASH |
| | | | | DGGKQALETVQRLLPVLCQAHGLTPEQVVAIASNIGGKQ |
| | | | | ALETVQALLPVLCQAHGLTPQQVVAIASNGGGKQALETV |
| | | | | QRLLPVLCQAHGLTPEQVVAIASHDGGKQALETVQRLLP |
| | | | | VLCQAHGLTP1QQVVAIASNGGGRPALESIVAQLSRPDP |
| | | | | ALAATNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGHKLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T001212 | HBV1530_T01.R1 | NN-HD-NI-NN- | (SEQ ID NO: 60) | (SEQ ID NO: 2) |
| | | NI-NN-NN-NG- | GCAGAGGTGAAGCGA | MGDPKKKRKVIDKETAAAKFERQHMDSIDIADLRTLGYS |
| | | NN-NI-NI-NN- | T# | QQQQEKIKPKVRSTVAQHHEALVGHGFTHAHIVALSQHP |
| | | HD-NN-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SNIGGKQALETVQALLPVLCQAHGLTPQQVVAIASNNGG |
| | | | | KQALETVQRLLPVLCQAHGLTPQQVVAIASNNGGKQALE |
| | | | | TVQRLLPVLCQAHGLTPQQVVAIASNGGGKQALETVQRL |
| | | | | LPVLCQAHGLTPQQVVAIASNNGGKQALETVQRLLPVLC |
| | | | | QAHGLTPEQVVAIASNIGGKQALETVQALLPVLCQAHGL |
| | | | | TPEQVVAIASNIGGKQALETVQALLPVLCQAHGLTPQQV |
| | | | | VAIASNNGGKQALETVQRLLPVLCQAHGLTPEQVVAIAS |
| | | | | HDGGKQALETVQRLLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T001213 | HBV1860_T0111 | NN-NG-NG-HD- | (SEQ ID NO: 61) | (SEQ ID NO: 3) |
| | | NI-NI-NN-HD- | GTTCAAGCCTCCAAG | MGDPKKKRKVIDYPYDVPDYAIDIADLRTLGYSQQQQEK |
| | | HD-NG-HD-HD- | T# | IKPKVRSTVAQHHEALVGHGFTHAHIVALSQHPAALGTV |
| | | NI-NI-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNGGGKQALETVQRLLPVLCQAHGLTPQQV |
| | | | | VAIASNGGGKQALETVQRLLPVLCQAHGLTPEQVVAIAS |
| | | | | HDGGKQALETVQRLLPVLCQAHGLTPEQVVAIASNIGGK |
| | | | | QALETVQALLPVLCQAHGLTPEQVVAIASNIGGKQALET |
| | | | | VQALLPVLCQAHGLTPQQVVAIASNNGGKQALETVQRLL |
| | | | | PVLCQAHGLTPEQVVAIASHDGGKQALETVQRLLPVLCQ |
| | | | | AHGLTPEQVVAIASHDGGKQALETVQRLLPVLCQAHGLT |
| | | | | PQQVVAIASNGGGKQALETVQRLLPVLCQAHGLTPEQVV |
| | | | | AIASHDGGKQALETVQRLLPVLCQAHGLTPEQVVAIASH |
| | | | | DGGKQALETVQRLLPVLCQAHGLTPEQVVAIASNIGGKQ |
| | | | | ALETVQALLPVLCQAHGLTPEQVVAIASNIGGKQALETV |
| | | | | QALLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T001213 | HBV1860_T01.R1 | NN-NG-HD-HD- | (SEQ ID NO: 62) | (SEQ ID NO: 4) |
| | | NI-NG-NN-HD- | GTCCATGCCCCAAAG | MGDPKKKRKVIDKETAAAKFERQHMDSIDIADLRTLGYS |
| | | HD-HD-HD-NI- | T# | QQQQEKIKPKVRSTVAQHHEALVGHGFTHAHIVALSQHP |
| | | NI-NI-NN-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPQQVVAIASNGGGKQALETVQRLLPVLCQAHG |
| | | | | LTPEQVVAIASHDGGKQALETVQRLLPVLCQAHGLTPEQ |
| | | | | VVAIASHDGGKQALETVQRLLPVLCQAHGLTPEQVVAIA |
| | | | | SNIGGKQALETVQALLPVLCQAHGLTPQQVVAIASNGGG |
| | | | | KQALETVQRLLPVLCQAHGLTPQQVVAIASNNGGKQALE |
| | | | | TVQRLLPVLCQAHGLTPEQVVAIASHDGGKQALETVQRL |
| | | | | LPVLCQAHGLTPEQVVAIASHDGGKQALETVQRLLPVLC |
| | | | | QAHGLTPEQVVAIASHDGGKQALETVQRLLPVLCQAHGL |
| | | | | TPEQVVAIASHDGGKQALETVQRLLPVLCQAHGLTPEQV |
| | | | | VAIASNIGGKQALETVQALLPVLCQAHGLTPEQVVAIAS |
| | | | | NIGGKQALETVQALLPVLCQAHGLTPEQVVAIASNIGGK |
| | | | | QALETVQALLPVLCQAHGLTPQQVVAIASNNGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T001214 | HBV2400_T01.L1 | HD-NN-HD-NI- | (SEQ ID NO: 63) | (SEQ ID NO: 5) |
| | | NN-NI-NI-NN- | CGCAGAAGATCTCAA | MGDPKKKRKVIDYPYDVPDYAIDIADLRTLGYSQQQQEK |
| | | NI-NG-HD-NG- | T# | IKPKVRSTVAQHHEALVGHGFTHAHIVALSQHPAALGTV |
| | | HD-NI-NI-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | NIGGKQALETVQALLPVLCQAHGLTPQQVVAIASNNGGK |
| | | | | QALETVQRLLPVLCQAHGLTPEQVVAIASNIGGKQALET |
| | | | | VQALLPVLCQAHGLTPEQVVAIASNIGGKQALETVQALL |
| | | | | PVLCQAHGLTPQQVVAIASNNGGKQALETVQRLLPVLCQ |
| | | | | AHGLTPEQVVAIASNIGGKQALETVQALLPVLCQAHGLT |
| | | | | PQQVVAIASNGGGKQALETVQRLLPVLCQAHGLTPEQVV |
| | | | | AIASHDGGKQALETVQRLLPVLCQAHGLTPQQVVAIASN |
| | | | | GGGKQALETVQRLLPVLCQAHGLTPEQVVAIASHDGGKQ |
| | | | | ALETVQRLLPVLCQAHGLTPEQVVAIASNIGGKQALETV |
| | | | | QALLPVLCQAHGLTPEQVVAIASNIGGKQALETVQALLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T001214 | HBV2400_T01.R1 | HD-HD-NI-NI- | (SEQ ID NO: 64) | (SEQ ID NO: 6) |
| | | NN-NN-NN-NI- | CCAAGGGATACTAAC | MGDPKKKRKVIDKETAAAKFERQHMDSIDIADLRTLGYS |
| | | NG-NI-HD-NG- | T# | QQQQEKIKPKVRSTVAQHHEALVGHGFTHAHIVALSQHP |
| | | NI-NI-HD-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASHDGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPEQ |
| | | | | VVAIASNIGGKQALETVQALLPVLCQAHGLTPQQVVAIA |
| | | | | SNNGGKQALETVQRLLPVLCQAHGLTPQQVVAIASNNGG |
| | | | | KQALETVQRLLPVLCQAHGLTPQQVVAIASNNGGKQALE |
| | | | | TVQRLLPVLCQAHGLTPEQVVAIASNIGGKQALETVQAL |
| | | | | LPVLCQAHGLTPQQVVAIASNGGGKQALETVQRLLPVLC |
| | | | | QAHGLTPEQVVAIASNIGGKQALETVQALLPVLCQAHGL |
| | | | | TPEQVVAIASHDGGKQALETVQRLLPVLCQAHGLTPQQV |
| | | | | VAIASNGGGKQALETVQRLLPVLCQAHGLTPEQVVAIAS |
| | | | | NIGGKQALETVQALLPVLCQAHGLTPEQVVAIASNIGGK |
| | | | | QALETVQALLPVLCQAHGLTPEQVVAIASHDGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T001215 | HBV180_T0111 | NN-NG-NG-NI- | (SEQ ID NO: 65) | (SEQ ID NO: 7) |
| | | HD-NI-NN-NN- | GTTACAGGCGGGGTT | MGDPKKKRKVIDYPYDVPDYAIDIADLRTLGYSQQQQEK |
| | | HD-NN-NN-NN- | T# | IKPKVRSTVAQHHEALVGHGFTHAHIVALSQHPAALGTV |
| | | NN-NG-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNGGGKQALETVQRLLPVLCQAHGLTPQQV |
| | | | | VAIASNGGGKQALETVQRLLPVLCQAHGLTPEQVVAIAS |
| | | | | NIGGKQALETVQALLPVLCQAHGLTPEQVVAIASHDGGK |
| | | | | QALETVQRLLPVLCQAHGLTPEQVVAIASNIGGKQALET |
| | | | | VQALLPVLCQAHGLTPQQVVAIASNNGGKQALETVQRLL |
| | | | | PVLCQAHGLTPQQVVAIASNNGGKQALETVQRLLPVLCQ |
| | | | | AHGLTPEQVVAIASHDGGKQALETVQRLLPVLCQAHGLT |
| | | | | PQQVVAIASNNGGKQALETVQRLLPVLCQAHGLTPQQVV |
| | | | | AIASNNGGKQALETVQRLLPVLCQAHGLTPQQVVAIASN |
| | | | | NGGKQALETVQRLLPVLCQAHGLTPQQVVAIASNNGGKQ |
| | | | | ALETVQRLLPVLCQAHGLTPQQVVAIASNGGGKQALETV |
| | | | | QRLLPVLCQAHGLTPQQVVAIASNGGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T001215 | HBV180_T01.R1 | NN-NG-NN-NN- | (SEQ ID NO: 66) | (SEQ ID NO: 8) |
| | | NG-NI-NG-NG- | GTGGTATTGTGAGGA | MGDPKKKRKVIDKETAAAKFERQHMDSIDIADLRTLGYS |
| | | NN-NG-NN-NI- | T# | QQQQEKIKPKVRSTVAQHHEALVGHGFTHAHIVALSQHP |
| | | NN-NN-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPQQVVAIASNGGGKQALETVQRLLPVLCQAHG |
| | | | | LTPQQVVAIASNNGGKQALETVQRLLPVLCQAHGLTPQQ |
| | | | | VVAIASNNGGKQALETVQRLLPVLCQAHGLTPQQVVAIA |
| | | | | SNGGGKQALETVQRLLPVLCQAHGLTPEQVVAIASNIGG |
| | | | | KQALETVQALLPVLCQAHGLTPQQVVAIASNGGGKQALE |
| | | | | TVQRLLPVLCQAHGLTPQQVVAIASNGGGKQALETVQRL |
| | | | | LPVLCQAHGLTPQQVVAIASNNGGKQALETVQRLLPVLC |
| | | | | QAHGLTPQQVVAIASNGGGKQALETVQRLLPVLCQAHGL |
| | | | | TPQQVVAIASNNGGKQALETVQRLLPVLCQAHGLTPEQV |
| | | | | VAIASNIGGKQALETVQALLPVLCQAHGLTPQQVVAIAS |
| | | | | NNGGKQALETVQRLLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002559 | HBV-core-1.L1 | NN-NG-NN-NN- | (SEQ ID NO: 67) | (SEQ ID NO: 9) |
| | | NI-NG-NG-HD- | GTGGATTCGCACTCC | MGDPKKKRKVIDYPYDVPDYAIDIADPIRSRTPSPAREL |
| | | NN-HD-NI-HD- | T# | LPGPQPDGVQPTADRGVSPPAGGPLDGLPARRTMSRTRL |
| | | NG-HD-HD-NG# | | PSPPAPSPAFSAGSFSDLLRQFDPSLFNTSLFDSLPPFG |
| | | | | AHHTEAATGEWDEVQSGLRAADAPPPTMRVAVTAARPPR |
| | | | | AKPAPRRRAAQPSDASPAAQVDLRTLGYSQQQQEKIKPK |
| | | | | VRSTVAQHHEALVGHGFTHAHIVALSQHPAALGTVAVKY |
| | | | | QDMIAALPEATHEAIVGVGKQWSGARALEALLTVAGELR |
| | | | | GPPLQLDTGQLLKIAKRGGVTAVEAVHAWRNALTGAPLN |
| | | | | LTPQQVVAIASNNGGKQALETVQRLLPVLCQAHGLTPQQ |
| | | | | VVAIASNGGGKQALETVQRLLPVLCQAHGLTPQQVVAIA |
| | | | | SNNGGKQALETVQRLLPVLCQAHGLTPQQVVAIASNNGG |
| | | | | KQALETVQRLLPVLCQAHGLTPEQVVAIASNIGGKQALE |
| | | | | TVQALLPVLCQAHGLTPQQVVAIASNGGGKQALETVQRL |
| | | | | LPVLCQAHGLTPQQVVAIASNGGGKQALETVQRLLPVLC |
| | | | | QAHGLTPEQVVAIASHDGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | NIGGKQALETVQALLPVLCQAHGLTPEQVVAIASHDGGK |
| | | | | QALETVQRLLPVLCQAHGLTPQQVVAIASNGGGKQALET |
| | | | | VQRLLPVLCQAHGLTPEQVVAIASHDGGKQALETVQRLL |
| | | | | PVLCQAHGLTPEQVVAIASHDGGKQALETVQRLLPVLCQ |
| | | | | AHGLTPQQVVAIASNGGGRPALESIVAQLSRPDPSGSGS |
| | | | | GGDPISRSQLVKSELEEKKSELRHKLKYVPHEYIELIEI |
| | | | | ARNSTQDRILEMKVMEFFMKVYGYRGKHLGGSRKPDGAI |
| | | | | YTVGSPIDYGVIVDTKAYSGGYNLPIGQADEMQRYVEEN |
| | | | | QTRNKHINPNEWWKVYPSSVTEFKFLFVSGHFKGNYKAQ |
| | | | | LTRLNHITNCNGAVLSVEELLIGGEMIKAGTLTLEEVRR |
| | | | | KFNNGEINFAAD |
| T002559 | HBV-core-1.R1 | NI-NN-NN-NN- | (SEQ ID NO: 68) | (SEQ ID NO: 10) |
| | | NN-HD-NI-NG- | AGGGGCATTTGGTGG | MGDPKKKRKVIDKETAAAKFERQHMDSIDIADPIRSRTP |
| | | NG-NG-NN-NN- | T# | SPARELLPGPQPDGVQPTADRGVSPPAGGPLDGLPARRT |
| | | NG-NN-NN-NG# | | MSRTRLPSPPAPSPAFSAGSFSDLLRQFDPSLFNTSLFD |
| | | | | SLPPFGAHHTEAATGEWDEVQSGLRAADAPPPTMRVAVT |
| | | | | AARPPRAKPAPRRRAAQPSDASPAAQVDLRTLGYSQQQQ |
| | | | | EKIKPKVRSTVAQHHEALVGHGFTHAHIVALSQHPAALG |
| | | | | TVAVKYQDMIAALPEATHEAIVGVGKQWSGARALEALLT |
| | | | | VAGELRGPPLQLDTGQLLKIAKRGGVTAVEAVHAWRNAL |
| | | | | TGAPLNLTPEQVVAIASNIGGKQALETVQALLPVLCQAH |
| | | | | GLTPQQVVAIASNNGGKQALETVQRLLPVLCQAHGLTPQ |
| | | | | QVVAIASNNGGKQALETVQRLLPVLCQAHGLTPQQVVAI |
| | | | | ASNNGGKQALETVQRLLPVLCQAHGLTPQQVVAIASNNG |
| | | | | GKQALETVQRLLPVLCQAHGLTPEQVVAIASHDGGKQAL |
| | | | | ETVQRLLPVLCQAHGLTPEQVVAIASNIGGKQALETVQA |
| | | | | LLPVLCQAHGLTPQQVVAIASNGGGKQALETVQRLLPVL |
| | | | | CQAHGLTPQQVVAIASNGGGKQALETVQRLLPVLCQAHG |
| | | | | LTPQQVVAIASNGGGKQALETVQRLLPVLCQAHGLTPQQ |
| | | | | VVAIASNNGGKQALETVQRLLPVLCQAHGLTPQQVVAIA |
| | | | | SNNGGKQALETVQRLLPVLCQAHGLTPQQVVAIASNGGG |
| | | | | KQALETVQRLLPVLCQAHGLTPQQVVAIASNNGGKQALE |
| | | | | TVQRLLPVLCQAHGLTPQQVVAIASNNGGKQALETVQRL |
| | | | | LPVLCQAHGLTPQQVVAIASNGGGRPALESIVAQLSRPD |
| | | | | PSGSGSGGDPISRSQLVKSELEEKKSELRHKLKYVPHEY |
| | | | | IELIEIARNSTQDRILEMKVMEFFMKVYGYRGKHLGGSR |
| | | | | KPDGAIYTVGSPIDYGVIVDTKAYSGGYNLPIGQADEMQ |
| | | | | RYVEENQTRNKHINPNEWWKVYPSSVTEFKFLFVSGHFK |
| | | | | GNYKAQLTRLNHITNCNGAVLSVEELLIGGEMIKAGTLT |
| | | | | LEEVRRKFNNGEINFAAD |
| T002560 | HBV-core-2.L1 | NN-HD-HD-HD- | (SEQ ID NO: 69) | (SEQ ID NO: 11) |
| | | HD-NG-NI-NG- | GCCCCTATCTTATCA | MGDPKKKRKVIDYPYDVPDYAIDIADPIRSRTPSPAREL |
| | | HD-NG-NG-NI- | T# | LPGPQPDGVQPTADRGVSPPAGGPLDGLPARRTMSRTRL |
| | | NG-HD-NI-NG# | | PSPPAPSPAFSAGSFSDLLRQFDPSLFNTSLFDSLPPFG |
| | | | | AHHTEAATGEWDEVQSGLRAADAPPPTMRVAVTAARPPR |
| | | | | AKPAPRRRAAQPSDASPAAQVDLRTLGYSQQQQEKIKPK |
| | | | | VRSTVAQHHEALVGHGFTHAHIVALSQHPAALGTVAVKY |
| | | | | QDMIAALPEATHEAIVGVGKQWSGARALEALLTVAGELR |
| | | | | GPPLQLDTGQLLKIAKRGGVTAVEAVHAWRNALTGAPLN |
| | | | | LTPQQVVAIASNNGGKQALETVQRLLPVLCQAHGLTPEQ |
| | | | | VVAIASHDGGKQALETVQRLLPVLCQAHGLTPEQVVAIA |
| | | | | SHDGGKQALETVQRLLPVLCQAHGLTPEQVVAIASHDGG |
| | | | | KQALETVQRLLPVLCQAHGLTPEQVVAIASHDGGKQALE |
| | | | | TVQRLLPVLCQAHGLTPQQVVAIASNGGGKQALETVQRL |
| | | | | LPVLCQAHGLTPEQVVAIASNIGGKQALETVQALLPVLC |
| | | | | QAHGLTPQQVVAIASNGGGKQALETVQRLLPVLCQAHGL |
| | | | | TPEQVVAIASHDGGKQALETVQRLLPVLCQAHGLTPQQV |
| | | | | VAIASNGGGKQALETVQRLLPVLCQAHGLTPQQVVAIAS |
| | | | | NGGGKQALETVQRLLPVLCQAHGLTPEQVVAIASNIGGK |
| | | | | QALETVQALLPVLCQAHGLTPQQVVAIASNGGGKQALET |
| | | | | VQRLLPVLCQAHGLTPEQVVAIASHDGGKQALETVQRLL |
| | | | | PVLCQAHGLTPEQVVAIASNIGGKQALETVQALLPVLCQ |
| | | | | AHGLTPQQVVAIASNGGGRPALESIVAQLSRPDPSGSGS |
| | | | | GGDPISRSQLVKSELEEKKSELRHKLKYVPHEYIELIEI |
| | | | | ARNSTQDRILEMKVMEFFMKVYGYRGKHLGGSRKPDGAI |
| | | | | YTVGSPIDYGVIVDTKAYSGGYNLPIGQADEMQRYVEEN |
| | | | | QTRNKHINPNEWWKVYPSSVTEFKFLFVSGHFKGNYKAQ |
| | | | | LTRLNHITNCNGAVLSVEELLIGGEMIKAGTLTLEEVRR |
| | | | | KFNNGEINFAAD |
| T002560 | HBV-core-2.R1 | HD-NN-NG-HD- | (SEQ ID NO: 70) | (SEQ ID NO: 12) |
| | | NG-NI-NI-HD- | CGTCTAACAACAGTA | MGDPKKKRKVIDKETAAAKFERQHMDSIDIADPIRSRTP |
| | | NI-NI-HD-NI- | T# | SPARELLPGPQPDGVQPTADRGVSPPAGGPLDGLPARRT |
| | | NN-NG-NI-NG# | | MSRTRLPSPPAPSPAFSAGSFSDLLRQFDPSLFNTSLFD |
| | | | | SLPPFGAHHTEAATGEWDEVQSGLRAADAPPPTMRVAVT |
| | | | | AARPPRAKPAPRRRAAQPSDASPAAQVDLRTLGYSQQQQ |
| | | | | EKIKPKVRSTVAQHHEALVGHGFTHAHIVALSQHPAALG |
| | | | | TVAVKYQDMIAALPEATHEAIVGVGKQWSGARALEALLT |
| | | | | VAGELRGPPLQLDTGQLLKIAKRGGVTAVEAVHAWRNAL |
| | | | | TGAPLNLTPEQVVAIASHDGGKQALETVQRLLPVLCQAH |
| | | | | GLTPQQVVAIASNNGGKQALETVQRLLPVLCQAHGLTPQ |
| | | | | QVVAIASNGGGKQALETVQRLLPVLCQAHGLTPEQVVAI |
| | | | | ASHDGGKQALETVQRLLPVLCQAHGLTPQQVVAIASNGG |
| | | | | GKQALETVQRLLPVLCQAHGLTPEQVVAIASNIGGKQAL |
| | | | | ETVQALLPVLCQAHGLTPEQVVAIASNIGGKQALETVQA |
| | | | | LLPVLCQAHGLTPEQVVAIASHDGGKQALETVQRLLPVL |
| | | | | CQAHGLTPEQVVAIASNIGGKQALETVQALLPVLCQAHG |
| | | | | LTPEQVVAIASNIGGKQALETVQALLPVLCQAHGLTPEQ |
| | | | | VVAIASHDGGKQALETVQRLLPVLCQAHGLTPEQVVAIA |
| | | | | SNIGGKQALETVQALLPVLCQAHGLTPQQVVAIASNNGG |
| | | | | KQALETVQRLLPVLCQAHGLTPQQVVAIASNGGGKQALE |
| | | | | TVQRLLPVLCQAHGLTPEQVVAIASNIGGKQALETVQAL |
| | | | | LPVLCQAHGLTPQQVVAIASNGGGRPALESIVAQLSRPD |
| | | | | PSGSGSGGDPISRSQLVKSELEEKKSELRHKLKYVPHEY |
| | | | | IELIEIARNSTQDRILEMKVMEFFMKVYGYRGKHLGGSR |
| | | | | KPDGAIYTVGSPIDYGVIVDTKAYSGGYNLPIGQADEMQ |
| | | | | RYVEENQTRNKHINPNEWWKVYPSSVTEFKFLFVSGHFK |
| | | | | GNYKAQLTRLNHITNCNGAVLSVEELLIGGEMIKAGTLT |
| | | | | LEEVRRKFNNGEINFAAD |
| T002561 | HBV-polym-1.L1 | NN-NG-HD-NG- | (SEQ ID NO: 71) | (SEQ ID NO: 13) |
| | | NN-HD-NN-NN- | GTCTGCGGCGTTTTA | MGDPKKKRKVIDYPYDVPDYAIDIADLRTLGYSQQQQEK |
| | | HD-NN-NG-NG- | T# | IKPKVRSTVAQHHEALVGHGFTHAHIVALSQHPAALGTV |
| | | NG-NG-NI-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNGGGKQALETVQRLLPVLCQAHGLTPEQV |
| | | | | VAIASHDGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASHDGGKQALET |
| | | | | VQRLLPVLCQAHGLTPQQVVAIASNNGGKQALETVQRLL |
| | | | | PVLCQAHGLTPQQVVAIASNNGGKQALETVQRLLPVLCQ |
| | | | | AHGLTPEQVVAIASHDGGKQALETVQRLLPVLCQAHGLT |
| | | | | PQQVVAIASNNGGKQALETVQRLLPVLCQAHGLTPQQVV |
| | | | | AIASNGGGKQALETVQRLLPVLCQAHGLTPQQVVAIASN |
| | | | | GGGKQALETVQRLLPVLCQAHGLTPQQVVAIASNGGGKQ |
| | | | | ALETVQRLLPVLCQAHGLTPQQVVAIASNGGGKQALETV |
| | | | | QRLLPVLCQAHGLTPEQVVAIASNIGGKQALETVQALLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002561 | HBV-polym-1.R1 | NN-NI-NN-NN- | (SEQ ID NO: 72) | (SEQ ID NO: 14) |
| | | HD-NI-NG-NI- | GAGGCATAGCAGCAG | MGDPKKKRKVIDKETAAAKFERQHMDSIDIADLRTLGYS |
| | | NN-HD-NI-NN- | T# | QQQQEKIKPKVRSTVAQHHEALVGHGFTHAHIVALSQHP |
| | | HD-NI-NN-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASNIGGKQALETVQALLPVLCQAHG |
| | | | | LTPQQVVAIASNNGGKQALETVQRLLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPEQVVAIASNIGG |
| | | | | KQALETVQALLPVLCQAHGLTPQQVVAIASNGGGKQALE |
| | | | | TVQRLLPVLCQAHGLTPEQVVAIASNIGGKQALETVQAL |
| | | | | LPVLCQAHGLTPQQVVAIASNNGGKQALETVQRLLPVLC |
| | | | | QAHGLTPEQVVAIASHDGGKQALETVQRLLPVLCQAHGL |
| | | | | TPEQVVAIASNIGGKQALETVQALLPVLCQAHGLTPQQV |
| | | | | VAIASNNGGKQALETVQRLLPVLCQAHGLTPEQVVAIAS |
| | | | | HDGGKQALETVQRLLPVLCQAHGLTPEQVVAIASNIGGK |
| | | | | QALETVQALLPVLCQAHGLTPQQVVAIASNNGGKQALET |
| | | | | VQRLLPVLCQAHGLTPQQVVAIASNGGGRPALESIVASR |
| | | | | PDPALAALTNDHLVALACLGGRPALDAVKKGLGDPISRS |
| | | | | QLVKSELEEKKSELRHKLKYVPHEYIELIEIARNSTQDR |
| | | | | ILEMKVMEFFMKVYGYRGKHLGGSRKPDGAIYTVGSPID |
| | | | | YGVIVDTKAYSGGYNLPIGQADEMQRYVEENQTRNKHIN |
| | | | | PNEWWKVYPSSVTEFKFLFVSGHFKGNYKAQLTRLNHIT |
| | | | | NCNGAVLSVEELLIGGEMIKAGTLTLEEVRRKFNNGEIN |
| | | | | FAAD |
| T002562 | HBV-polym-2.L1 | HD-HD-HD-NG- | (SEQ ID NO: 73) | (SEQ ID NO: 15) |
| | | HD-NN-HD-HD- | CCCTCGCCTCGCAGA | MGDPKKKRKVIDYPYDVPDYAIDIADPIRSRTPSPAREL |
| | | NG-HD-NN-HD- | T# | LPGPQPDGVQPTADRGVSPPAGGPLDGLPARRTMSRTRL |
| | | NI-NN-NI-NG# | | PSPPAPSPAFSAGSFSDLLRQFDPSLFNTSLFDSLPPFG |
| | | | | AHHTEAATGEWDEVQSGLRAADAPPPTMRVAVTAARPPR |
| | | | | AKPAPRRRAAQPSDASPAAQVDLRTLGYSQQQQEKIKPK |
| | | | | VRSTVAQHHEALVGHGFTHAHIVALSQHPAALGTVAVKY |
| | | | | QDMIAALPEATHEAIVGVGKQWSGARALEALLTVAGELR |
| | | | | GPPLQLDTGQLLKIAKRGGVTAVEAVHAWRNALTGAPLN |
| | | | | LTPEQVVAIASHDGGKQALETVQRLLPVLCQAHGLTPEQ |
| | | | | VVAIASHDGGKQALETVQRLLPVLCQAHGLTPEQVVAIA |
| | | | | SHDGGKQALETVQRLLPVLCQAHGLTPQQVVAIASNGGG |
| | | | | KQALETVQRLLPVLCQAHGLTPEQVVAIASHDGGKQALE |
| | | | | TVQRLLPVLCQAHGLTPQQVVAIASNNGGKQALETVQRL |
| | | | | LPVLCQAHGLTPEQVVAIASHDGGKQALETVQRLLPVLC |
| | | | | QAHGLTPEQVVAIASHDGGKQALETVQRLLPVLCQAHGL |
| | | | | TPQQVVAIASNGGGKQALETVQRLLPVLCQAHGLTPEQV |
| | | | | VAIASHDGGKQALETVQRLLPVLCQAHGLTPQQVVAIAS |
| | | | | NNGGKQALETVQRLLPVLCQAHGLTPEQVVAIASHDGGK |
| | | | | QALETVQRLLPVLCQAHGLTPEQVVAIASNIGGKQALET |
| | | | | VQALLPVLCQAHGLTPQQVVAIASNNGGKQALETVQRLL |
| | | | | PVLCQAHGLTPEQVVAIASNIGGKQALETVQALLPVLCQ |
| | | | | AHGLTPQQVVAIASNGGGRPALESIVAQLSRPDPSGSGS |
| | | | | GGDPISRSQLVKSELEEKKSELRHKLKYVPHEYIELIEI |
| | | | | ARNSTQDRILEMKVMEFFMKVYGYRGKHLGGSRKPDGAI |
| | | | | YTVGSPIDYGVIVDTKAYSGGYNLPIGQADEMQRYVEEN |
| | | | | QTRNKHINPNEWWKVYPSSVTEFKFLFVSGHFKGNYKAQ |
| | | | | LTRLNHITNCNGAVLSVEELLIGGEMIKAGTLTLEEVRR |
| | | | | KFNNGEINFAAD |
| T002562 | HBV-polym-2.R1 | HD-NG-NG-HD- | (SEQ ID NO: 74) | (SEQ ID NO: 16) |
| | | NG-NN-HD-NN- | CTTCTGCGACGCGGC | MGDPKKKRKVIDKETAAAKFERQHMDSIDIADPIRSRTP |
| | | NI-HD-NN-HD- | T# | SPARELLPGPQPDGVQPTADRGVSPPAGGPLDGLPARRT |
| | | NN-NN-HD-NG# | | MSRTRLPSPPAPSPAFSAGSFSDLLRQFDPSLFNTSLFD |
| | | | | SLPPFGAHHTEAATGEWDEVQSGLRAADAPPPTMRVAVT |
| | | | | AARPPRAKPAPRRRAAQPSDASPAAQVDLRTLGYSQQQQ |
| | | | | EKIKPKVRSTVAQHHEALVGHGFTHAHIVALSQHPAALG |
| | | | | TVAVKYQDMIAALPEATHEAIVGVGKQWSGARALEALLT |
| | | | | VAGELRGPPLQLDTGQLLKIAKRGGVTAVEAVHAWRNAL |
| | | | | TGAPLNLTPEQVVAIASHDGGKQALETVQRLLPVLCQAH |
| | | | | GLTPQQVVAIASNGGGKQALETVQRLLPVLCQAHGLTPQ |
| | | | | QVVAIASNGGGKQALETVQRLLPVLCQAHGLTPEQVVAI |
| | | | | ASHDGGKQALETVQRLLPVLCQAHGLTPQQVVAIASNGG |
| | | | | GKQALETVQRLLPVLCQAHGLTPQQVVAIASNNGGKQAL |
| | | | | ETVQRLLPVLCQAHGLTPEQVVAIASHDGGKQALETVQR |
| | | | | LLPVLCQAHGLTPQQVVAIASNNGGKQALETVQRLLPVL |
| | | | | CQAHGLTPEQVVAIASNIGGKQALETVQALLPVLCQAHG |
| | | | | LTPEQVVAIASHDGGKQALETVQRLLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPQQVVAIASNNGG |
| | | | | KQALETVQRLLPVLCQAHGLTPQQVVAIASNNGGKQALE |
| | | | | TVQRLLPVLCQAHGLTPEQVVAIASHDGGKQALETVQRL |
| | | | | LPVLCQAHGLTPQQVVAIASNGGGRPALESIVAQLSRPD |
| | | | | PSGSGSGGDPISRSQLVKSELEEKKSELRHKLKYVPHEY |
| | | | | IELIEIARNSTQDRILEMKVMEFFMKVYGYRGKHLGGSR |
| | | | | KPDGAIYTVGSPIDYGVIVDTKAYSGGYNLPIGQADEMQ |
| | | | | RYVEENQTRNKHINPNEWWKVYPSSVTEFKFLFVSGHFK |
| | | | | GNYKAQLTRLNHITNCNGAVLSVEELLIGGEMIKAGTLT |
| | | | | LEEVRRKFNNGEINFAAD |
| T002563 | HBV-HBX-1.L1 | NG-HD-NG-HD- | (SEQ ID NO: 75) | (SEQ ID NO: 17) |
| | | NI-NG-HD-NG- | TCTCATCTGCCGGTC | MGDPKKKRKVIDYPYDVPDYAIDIADLRTLGYSQQQQEK |
| | | NN-HD-HD-NN- | T# | IKPKVRSTVAQHHEALVGHGFTHAHIVALSQHPAALGTV |
| | | NN-NG-HD-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPEQVVAIASHDGGKQALETVQRLLPVLCQAHGLTPQQV |
| | | | | VAIASNGGGKQALETVQRLLPVLCQAHGLTPEQVVAIAS |
| | | | | HDGGKQALETVQRLLPVLCQAHGLTPEQVVAIASNIGGK |
| | | | | QALETVQALLPVLCQAHGLTPQQVVAIASNGGGKQALET |
| | | | | VQRLLPVLCQAHGLTPEQVVAIASHDGGKQALETVQRLL |
| | | | | PVLCQAHGLTPQQVVAIASNGGGKQALETVQRLLPVLCQ |
| | | | | AHGLTPQQVVAIASNNGGKQALETVQRLLPVLCQAHGLT |
| | | | | PEQVVAIASHDGGKQALETVQRLLPVLCQAHGLTPEQVV |
| | | | | AIASHDGGKQALETVQRLLPVLCQAHGLTPQQVVAIASN |
| | | | | NGGKQALETVQRLLPVLCQAHGLTPQQVVAIASNNGGKQ |
| | | | | ALETVQRLLPVLCQAHGLTPQQVVAIASNGGGKQALETV |
| | | | | QRLLPVLCQAHGLTPEQVVAIASHDGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002563 | HBV-HBX-1.R1 | NN-HD-NI-NI- | (SEQ ID NO: 76) | (SEQ ID NO: 18) |
| | | HD-NN-NG-NN- | GCAACGTGCAGAGGT | MGDPKKKRKVIDKETAAAKFERQHMDSIDIADLRTLGYS |
| | | HD-NI-NN-NI- | T# | QQQQEKIKPKVRSTVAQHHEALVGHGFTHAHIVALSQHP |
| | | NN-NN-NG-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPEQ |
| | | | | VVAIASNIGGKQALETVQALLPVLCQAHGLTPEQVVAIA |
| | | | | SHDGGKQALETVQRLLPVLCQAHGLTPQQVVAIASNNGG |
| | | | | KQALETVQRLLPVLCQAHGLTPQQVVAIASNGGGKQALE |
| | | | | TVQRLLPVLCQAHGLTPQQVVAIASNNGGKQALETVQRL |
| | | | | LPVLCQAHGLTPEQVVAIASHDGGKQALETVQRLLPVLC |
| | | | | QAHGLTPEQVVAIASNIGGKQALETVQALLPVLCQAHGL |
| | | | | TPQQVVAIASNNGGKQALETVQRLLPVLCQAHGLTPEQV |
| | | | | VAIASNIGGKQALETVQALLPVLCQAHGLTPQQVVAIAS |
| | | | | NNGGKQALETVQRLLPVLCQAHGLTPQQVVAIASNNGGK |
| | | | | QALETVQRLLPVLCQAHGLTPQQVVAIASNGGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002564 | HBV-HBX-2.L1 | NG-NG-NI-HD- | (SEQ ID NO: 77) | (SEQ ID NO: 19) |
| | | NN-HD-NN-NN- | TTACGCGGTCTCCCC | MGDPKKKRKVIDYPYDVPDYAIDIADLRTLGYSQQQQEK |
| | | NG-HD-NG-HD- | T# | IKPKVRSTVAQHHEALVGHGFTHAHIVALSQHPAALGTV |
| | | HD-HD-HD-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPQQVVAIASNGGGKQALETVQRLLPVLCQAHGLTPEQV |
| | | | | VAIASNIGGKQALETVQALLPVLCQAHGLTPEQVVAIAS |
| | | | | HDGGKQALETVQRLLPVLCQAHGLTPQQVVAIASNNGGK |
| | | | | QALETVQRLLPVLCQAHGLTPEQVVAIASHDGGKQALET |
| | | | | VQRLLPVLCQAHGLTPQQVVAIASNNGGKQALETVQRLL |
| | | | | PVLCQAHGLTPQQVVAIASNNGGKQALETVQRLLPVLCQ |
| | | | | AHGLTPQQVVAIASNGGGKQALETVQRLLPVLCQAHGLT |
| | | | | PEQVVAIASHDGGKQALETVQRLLPVLCQAHGLTPQQVV |
| | | | | AIASNGGGKQALETVQRLLPVLCQAHGLTPEQVVAIASH |
| | | | | DGGKQALETVQRLLPVLCQAHGLTPEQVVAIASHDGGKQ |
| | | | | ALETVQRLLPVLCQAHGLTPEQVVAIASHDGGKQALETV |
| | | | | QRLLPVLCQAHGLTPEQVVAIASHDGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002564 | HBV-HBX-2.R1 | NN-HD-NI-HD- | (SEQ ID NO: 78) | (SEQ ID NO: 20) |
| | | NI-HD-NN-NN- | GCACACGGACCGGCA | MGDPKKKRKVIDKETAAAKFERQHMDSIDIADLRTLGYS |
| | | NI-HD-HD-NN- | T# | QQQQEKIKPKVRSTVAQHHEALVGHGFTHAHIVALSQHP |
| | | NN-HD-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPEQ |
| | | | | VVAIASHDGGKQALETVQRLLPVLCQAHGLTPEQVVAIA |
| | | | | SNIGGKQALETVQALLPVLCQAHGLTPEQVVAIASHDGG |
| | | | | KQALETVQRLLPVLCQAHGLTPQQVVAIASNNGGKQALE |
| | | | | TVQRLLPVLCQAHGLTPQQVVAIASNNGGKQALETVQRL |
| | | | | LPVLCQAHGLTPEQVVAIASNIGGKQALETVQALLPVLC |
| | | | | QAHGLTPEQVVAIASHDGGKQALETVQRLLPVLCQAHGL |
| | | | | TPEQVVAIASHDGGKQALETVQRLLPVLCQAHGLTPQQV |
| | | | | VAIASNNGGKQALETVQRLLPVLCQAHGLTPQQVVAIAS |
| | | | | NNGGKQALETVQRLLPVLCQAHGLTPEQVVAIASHDGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002569 | mm_F7_exon1-3.L1 | HD-NG-HD-NG- | (SEQ ID NO: 79) | (SEQ ID NO: 21) |
| | | NN-HD-NG-NG- | CTCTGCTTTCTGCTC | MGDPKKKRKVIDYPYDVPDYAIDIADLRTLGYSQQQQEK |
| | | NG-HD-NG-NN- | T# | IKPKVRSTVAQHHEALVGHGFTHAHIVALSQHPAALGTV |
| | | HD-NG-HD-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPEQV |
| | | | | VAIASHDGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASHDGGKQALET |
| | | | | VQRLLPVLCQAHGLTPQQVVAIASNGGGKQALETVQRLL |
| | | | | PVLCQAHGLTPQQVVAIASNGGGKQALETVQRLLPVLCQ |
| | | | | AHGLTPQQVVAIASNGGGKQALETVQRLLPVLCQAHGLT |
| | | | | PEQVVAIASHDGGKQALETVQRLLPVLCQAHGLTPQQVV |
| | | | | AIASNGGGKQALETVQRLLPVLCQAHGLTPQQVVAIASN |
| | | | | NGGKQALETVQRLLPVLCQAHGLTPEQVVAIASHDGGKQ |
| | | | | ALETVQRLLPVLCQAHGLTPQQVVAIASNGGGKQALETV |
| | | | | QRLLPVLCQAHGLTPEQVVAIASHDGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002569 | mm_F7_exon1-3.R1 | NI-NG-NI-HD- | (SEQ ID NO: 80) | (SEQ ID NO: 22) |
| | | HD-NG-NN-HD- | ATACCTGCAGTCCCT | MGDPKKKRKVIDKETAAAKFERQHMDSIDIADLRTLGYS |
| | | NI-NN-NG-HD- | T# | QQQQEKIKPKVRSTVAQHHEALVGHGFTHAHIVALSQHP |
| | | HD-HD-NG-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASNIGGKQALETVQALLPVL |
| | | | | CQAHGLTPQQVVAIASNGGGKQALETVQRLLPVLCQAHG |
| | | | | LTPEQVVAIASNIGGKQALETVQALLPVLCQAHGLTPEQ |
| | | | | VVAIASHDGGKQALETVQRLLPVLCQAHGLTPEQVVAIA |
| | | | | SHDGGKQALETVQRLLPVLCQAHGLTPQQVVAIASNGGG |
| | | | | KQALETVQRLLPVLCQAHGLTPQQVVAIASNNGGKQALE |
| | | | | TVQRLLPVLCQAHGLTPEQVVAIASHDGGKQALETVQRL |
| | | | | LPVLCQAHGLTPEQVVAIASNIGGKQALETVQALLPVLC |
| | | | | QAHGLTPQQVVAIASNNGGKQALETVQRLLPVLCQAHGL |
| | | | | TPQQVVAIASNGGGKQALETVQRLLPVLCQAHGLTPEQV |
| | | | | VAIASHDGGKQALETVQRLLPVLCQAHGLTPEQVVAIAS |
| | | | | HDGGKQALETVQRLLPVLCQAHGLTPEQVVAIASHDGGK |
| | | | | QALETVQRLLPVLCQAHGLTPQQVVAIASNGGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002575 | mm_F7_exon6-2.L1 | HD-NG-HD-NG- | (SEQ ID NO: 81) | (SEQ ID NO: 23) |
| | | HD-NG-NN-NI- | CTCTCTGACTTCTGT | MGDPKKKRKVIDYPYDVPDYAIDIADLRTLGYSQQQQEK |
| | | HD-NG-NG-HD- | T# | IKPKVRSTVAQHHEALVGHGFTHAHIVALSQHPAALGTV |
| | | NG-NN-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPEQV |
| | | | | VAIASHDGGKQALETVQRLLPVLCQAHGLTPQQVVAIAS |
| | | | | NGGGKQALETVQRLLPVLCQAHGLTPEQVVAIASHDGGK |
| | | | | QALETVQRLLPVLCQAHGLTPQQVVAIASNGGGKQALET |
| | | | | VQRLLPVLCQAHGLTPQQVVAIASNNGGKQALETVQRLL |
| | | | | PVLCQAHGLTPEQVVAIASNIGGKQALETVQALLPVLCQ |
| | | | | AHGLTPEQVVAIASHDGGKQALETVQRLLPVLCQAHGLT |
| | | | | PQQVVAIASNGGGKQALETVQRLLPVLCQAHGLTPQQVV |
| | | | | AIASNGGGKQALETVQRLLPVLCQAHGLTPEQVVAIASH |
| | | | | DGGKQALETVQRLLPVLCQAHGLTPQQVVAIASNGGGKQ |
| | | | | ALETVQRLLPVLCQAHGLTPQQVVAIASNNGGKQALETV |
| | | | | QRLLPVLCQAHGLTPQQVVAIASNGGGKQALETVQRLLP |
| | | | | VLCQAHGLTPQQVVAIASNGGGRPALESIVAQLSRPDPS |
| | | | | GSGSGGDPISRSQLVKSELEEKKSELRHKLKYVPHEYIE |
| | | | | LIEIARNSTQDRILEMKVMEFFMKVYGYRGKHLGGSRKP |
| | | | | DGAIYTVGSPIDYGVIVDTKAYSGGYNLPIGQADEMQRY |
| | | | | VEENQTRNKHINPNEWWKVYPSSVTEFKFLFVSGHFKGN |
| | | | | YKAQLTRLNHITNCNGAVLSVEELLIGGEMIKAGTLTLE |
| | | | | EVRRKFNNGEINFAAD |
| T002575 | mm_F7_exon6-2.R1 | NG-HD-NG-HD- | (SEQ ID NO: 82) | (SEQ ID NO: 24) |
| | | HD-HD-NI-HD- | TCTCCCACACGGGTA | MGDPKKKRKVIDKETAAAKFERQHMDSIDIADLRTLGYS |
| | | NI-HD-NN-NN- | T# | QQQQEKIKPKVRSTVAQHHEALVGHGFTHAHIVALSQHP |
| | | NN-NG-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNGGGKQALETVQRLLPVL |
| | | | | CQAHGLTPEQVVAIASHDGGKQALETVQRLLPVLCQAHG |
| | | | | LTPQQVVAIASNGGGKQALETVQRLLPVLCQAHGLTPEQ |
| | | | | VVAIASHDGGKQALETVQRLLPVLCQAHGLTPEQVVAIA |
| | | | | SHDGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPEQVVAIASHDGGKQALETVQRL |
| | | | | LPVLCQAHGLTPEQVVAIASNIGGKQALETVQALLPVLC |
| | | | | QAHGLTPEQVVAIASHDGGKQALETVQRLLPVLCQAHGL |
| | | | | TPQQVVAIASNNGGKQALETVQRLLPVLCQAHGLTPQQV |
| | | | | VAIASNNGGKQALETVQRLLPVLCQAHGLTPQQVVAIAS |
| | | | | NNGGKQALETVQRLLPVLCQAHGLTPQQVVAIASNGGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPSGSGSGGDPISRSQLVKSELEEKKSELRHKLKYV |
| | | | | PHEYIELIEIARNSTQDRILEMKVMEFFMKVYGYRGKHL |
| | | | | GGSRKPDGAIYTVGSPIDYGVIVDTKAYSGGYNLPIGQA |
| | | | | DEMQRYVEENQTRNKHINPNEWWKVYPSSVTEFKFLFVS |
| | | | | GHFKGNYKAQLTRLNHITNCNGAVLSVEELLIGGEMIKA |
| | | | | GTLTLEEVRRKFNNGEINFAAD |
| T002657 | hAPOC3_Ex3A-L1 | HD-NG-NN-NG- | (SEQ ID NO: 83) | (SEQ ID NO: 25) |
| | | NG-NN-HD-NG- | CTGTTGCTTCCCCTG | MGDPKKKRKVIDYPYDVPDYAIDIADLRTLGYSQQQQEK |
| | | NG-HD-HD-HD- | T# | IKPKVRSTVAQHHEALVGHGFTHAHIVALSQHPAALGTV |
| | | HD-NG-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPQQV |
| | | | | VAIASNNGGKQALETVQRLLPVLCQAHGLTPQQVVAIAS |
| | | | | NGGGKQALETVQRLLPVLCQAHGLTPQQVVAIASNGGGK |
| | | | | QALETVQRLLPVLCQAHGLTPQQVVAIASNNGGKQALET |
| | | | | VQRLLPVLCQAHGLTPEQVVAIASHDGGKQALETVQRLL |
| | | | | PVLCQAHGLTPQQVVAIASNGGGKQALETVQRLLPVLCQ |
| | | | | AHGLTPQQVVAIASNGGGKQALETVQRLLPVLCQAHGLT |
| | | | | PEQVVAIASHDGGKQALETVQRLLPVLCQAHGLTPEQVV |
| | | | | AIASHDGGKQALETVQRLLPVLCQAHGLTPEQVVAIASH |
| | | | | DGGKQALETVQRLLPVLCQAHGLTPEQVVAIASHDGGKQ |
| | | | | ALETVQRLLPVLCQAHGLTPQQVVAIASNGGGKQALETV |
| | | | | QRLLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002657 | hAPOC3_Ex3A-R1 | NN-NI-NI-NN- | (SEQ ID NO: 84) | (SEQ ID NO: 26) |
| | | HD-HD-NI-NG- | GAAGCCATCGGTCAC | MGDPKKKRKVIDKETAAAKFERQHMDSIDIADLRTLGYS |
| | | HD-NN-NN-NG- | T# | QQQQEKIKPKVRSTVAQHHEALVGHGFTHAHIVALSQHP |
| | | HD-NI-HD-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASNIGGKQALETVQALLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPQQVVAIASNGGGKQALETVQRL |
| | | | | LPVLCQAHGLTPEQVVAIASHDGGKQALETVQRLLPVLC |
| | | | | QAHGLTPQQVVAIASNNGGKQALETVQRLLPVLCQAHGL |
| | | | | TPQQVVAIASNNGGKQALETVQRLLPVLCQAHGLTPQQV |
| | | | | VAIASNGGGKQALETVQRLLPVLCQAHGLTPEQVVAIAS |
| | | | | HDGGKQALETVQRLLPVLCQAHGLTPEQVVAIASNIGGK |
| | | | | QALETVQALLPVLCQAHGLTPEQVVAIASHDGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002658 | hAPOC3_Ex3B-L1 | NN-NI-NI-NI- | (SEQ ID NO: 85) | (SEQ ID NO: 27) |
| | | NN-NI-HD-NG- | GAAAGACTACTGGAG | MGDPKKKRKVIDYPYDVPDYAIDIADLRTLGYSQQQQEK |
| | | NI-HD-NG-NN- | T# | IKPKVRSTVAQHHEALVGHGFTHAHIVALSQHPAALGTV |
| | | NN-NI-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPEQVVAIASNIGGKQALETVQALLPVLCQAHGLTPEQV |
| | | | | VAIASNIGGKQALETVQALLPVLCQAHGLTPEQVVAIAS |
| | | | | NIGGKQALETVQALLPVLCQAHGLTPQQVVAIASNNGGK |
| | | | | QALETVQRLLPVLCQAHGLTPEQVVAIASNIGGKQALET |
| | | | | VQALLPVLCQAHGLTPEQVVAIASHDGGKQALETVQRLL |
| | | | | PVLCQAHGLTPQQVVAIASNGGGKQALETVQRLLPVLCQ |
| | | | | AHGLTPEQVVAIASNIGGKQALETVQALLPVLCQAHGLT |
| | | | | PEQVVAIASHDGGKQALETVQRLLPVLCQAHGLTPQQVV |
| | | | | AIASNGGGKQALETVQRLLPVLCQAHGLTPQQVVAIASN |
| | | | | NGGKQALETVQRLLPVLCQAHGLTPQQVVAIASNNGGKQ |
| | | | | ALETVQRLLPVLCQAHGLTPEQVVAIASNIGGKQALETV |
| | | | | QALLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002658 | hAPOC3_Ex3B-R1 | HD-HD-HD-NI- | (SEQ ID NO: 86) | (SEQ ID NO: 28) |
| | | NN-NI-NI-HD- | CCCAGAACTCAGAGA | MGDPKKKRKVIDKETAAAKFERQHMDSIDIADLRTLGYS |
| | | NG-HD-NI-NN- | T# | QQQQEKIKPKVRSTVAQHHEALVGHGFTGQLLKIAKRGG |
| | | NI-NN-NI-NG# | | VTAVEAVHAWRNALTGAPLNLTPEQVVAIASHDGGKQAL |
| | | | | ETVQRLLPVLCQAHGLTPEQVVAIASHDGGKQALETVQR |
| | | | | LLPVLCQAHGLTPEQVVAIASHDGGKQALETVQRLLPVL |
| | | | | CQAHGLTPEQVVAIASNIGGKQALETVQALLPVLCQAHG |
| | | | | LTPQQVVAIASNNGGKQALETVQRLLPVLCQAHGLTPEQ |
| | | | | VVAIASNIGGKQALETVQALLPVLCQAHGLTPEQVVAIA |
| | | | | SNIGGKQALETVQALLPVLCQAHGLTPEQVVAIASHDGG |
| | | | | KQALETVQRLLPVLCQAHGLTPQQVVAIASNGGGKQALE |
| | | | | TVQRLLPVLCQAHGLTPEQVVAIASHDGGKQALETVQRL |
| | | | | LPVLCQAHGLTPEQVVAIASNIGGKQALETVQALLPVLC |
| | | | | QAHGLTPQQVVAIASNNGGKQALETVQRLLPVLCQAHGL |
| | | | | TPEQVVAIASNIGGKQALETVQALLPVLCQAHGLTPQQV |
| | | | | VAIASNNGGKQALETVQRLLPVLCQAHGLTPEQVVAIAS |
| | | | | NIGGKQALETVQALLPVLCQAHGLTPQQVVAIASNGGGR |
| | | | | PALESIVAQLSRPDPALAALTNDHLVALACLGGRPALDA |
| | | | | VKKGLGDPISRSQLVKSELEEKKSELRHKLKYVPHEYIE |
| | | | | LIEIARNSTQDRILEMKVMEFFMKVYGYRGKHLGGSRKP |
| | | | | DGAIYTVGSPIDYGVIVDTKAYSGGYNLPIGQADEMQRY |
| | | | | VEENQTRNKHINPNEWWKVYPSSVTEFKFLFVSGHFKGN |
| | | | | YKAQLTRLNHITNCNGAVLSVEELLIGGEMIKAGTLTLE |
| | | | | EVRRKFNNGEINFAAD |
| T002671 | hAPOC3_Ex3_AN-R1 | NN-NI-NI-NN- | (SEQ ID NO: 87) | (SEQ ID NO: 29) |
| | | HD-HD-NI-NG- | GAAGCCAT5GGTCAC | MGDPKKKRKVIDKETAAAKFERQHMDSIDIADLRTLGYS |
| | | N-NN-NN-NG- | T# | QQQQEKIKPKVRSTVAQHHEALVGHGFTHAHIVALSQHP |
| | | HD-NI-HD-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASNIGGKQALETVQALLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPQQVVAIASNGGGKQALETVQRL |
| | | | | LPVLCQAHGLTPQQVVAIASNGGKQALETVQRLLPVLCQ |
| | | | | AHGLTPQQVVAIASNNGGKQALETVQRLLPVLCQAHGLT |
| | | | | PQQVVAIASNNGGKQALETVQRLLPVLCQAHGLTPQQVV |
| | | | | AIASNGGGKQALETVQRLLPVLCQAHGLTPEQVVAIASH |
| | | | | DGGKQALETVQRLLPVLCQAHGLTPEQVVAIASNIGGKQ |
| | | | | ALETVQALLPVLCQAHGLTPEQVVAIASHDGGKQALETV |
| | | | | QRLLPVLCQAHGLTPQQVVAIASNGGGRPALESIVAQLS |
| | | | | RPDPALAALTNDHLVALACLGGRPALDAVKKGLGDPISR |
| | | | | SQLVKSELEEKKSELRHKLKYVPHEYIELIEIARNSTQD |
| | | | | RILEMKVMEFFMKVYGYRGKHLGGSRKPDGAIYTVGSPI |
| | | | | DYGVIVDTKAYSGGYNLPIGQADEMQRYVEENQTRNKHI |
| | | | | NPNEWWKVYPSSVTEFKFLFVSGHFKGNYKAQLTRLNHI |
| | | | | TNCNGAVLSVEELLIGGEMIKAGTLTLEEVRRKFNNGEI |
| | | | | NFAAD |
| T002671 | hAPOC3_Ex3A-Li | HD-NG-NN-NG- | (SEQ ID NO: 88) | (SEQ ID NO: 30) |
| | | NG-NN-HD-NG- | CTGTTGCTTCCCCTG | MGDPKKKRKVIDYPYDVPDYAIDIADLRTLGYSQQQQEK |
| | | NG-HD-HD-HD- | T# | IKPKVRSTVAQHHEALVGHGFTHAHIVALSQHPAALGTV |
| | | HD-NG-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPQQV |
| | | | | VAIASNNGGKQALETVQRLLPVLCQAHGLTPQQVVAIAS |
| | | | | NGGGKQALETVQRLLPVLCQAHGLTPQQVVAIASNGGGK |
| | | | | QALETVQRLLPVLCQAHGLTPQQVVAIASNNGGKQALET |
| | | | | VQRLLPVLCQAHGLTPEQVVAIASHDGGKQALETVQRLL |
| | | | | PVLCQAHGLTPQQVVAIASNGGGKQALETVQRLLPVLCQ |
| | | | | AHGLTPQQVVAIASNGGGKQALETVQRLLPVLCQAHGLT |
| | | | | PEQVVAIASHDGGKQALETVQRLLPVLCQAHGLTPEQVV |
| | | | | AIASHDGGKQALETVQRLLPVLCQAHGLTPEQVVAIASH |
| | | | | DGGKQALETVQRLLPVLCQAHGLTPEQVVAIASHDGGKQ |
| | | | | ALETVQRLLPVLCQAHGLTPQQVVAIASNGGGKQALETV |
| | | | | QRLLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002659 | hTTR_Ex1A-L1 | HD-NG-NG-NN- | (SEQ ID NO: 89) | (SEQ ID NO: 31) |
| | | NN-HD-NI-NN- | CTTGGCAGGATGGCT | MGDPKKKRKVIDYPYDVPDYAIDIADLRTLGYSQQQQEK |
| | | NN-NI-NG-NN- | T# | IKPKVRSTVAQHHEALVGHGFTHAHIVALSQHPAALGTV |
| | | NN-HD-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPQQV |
| | | | | VAIASNGGGKQALETVQRLLPVLCQAHGLTPQQVVAIAS |
| | | | | NNGGKQALETVQRLLPVLCQAHGLTPQQVVAIASNNGGK |
| | | | | QALETVQRLLPVLCQAHGLTPEQVVAIASHDGGKQALET |
| | | | | VQRLLPVLCQAHGLTPEQVVAIASNIGGKQALETVQALL |
| | | | | PVLCQAHGLTPQQVVAIASNNGGKQALETVQRLLPVLCQ |
| | | | | AHGLTPQQVVAIASNNGGKQALETVQRLLPVLCQAHGLT |
| | | | | PEQVVAIASNIGGKQALETVQALLPVLCQAHGLTPQQVV |
| | | | | AIASNGGGKQALETVQRLLPVLCQAHGLTPQQVVAIASN |
| | | | | NGGKQALETVQRLLPVLCQAHGLTPQQVVAIASNNGGKQ |
| | | | | ALETVQRLLPVLCQAHGLTPEQVVAIASHDGGKQALETV |
| | | | | QRLLPVLCQAHGLTPQQVVAIASNGGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002659 | hTTR_Ex1A-R1 | HD-HD-NI-NN- | (SEQ ID NO: 90) | (SEQ ID NO: 32) |
| | | HD-NI-NI-NN- | CCAGCAAGGCAGAGG | MGDPKKKRKVIDKETAAAKFERQHMDSIDIADLRTLGYS |
| | | NN-HD-NI-NN- | T# | QQQQEKIKPKVRSTVAQHHEALVGHGFTHAHIVALSQHP |
| | | NI-NN-NN-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASHDGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPEQVVAIASNIGG |
| | | | | KQALETVQALLPVLCQAHGLTPEQVVAIASNIGGKQALE |
| | | | | TVQALLPVLCQAHGLTPQQVVAIASNNGGKQALETVQRL |
| | | | | LPVLCQAHGLTPQQVVAIASNNGGKQALETVQRLLPVLC |
| | | | | QAHGLTPEQVVAIASHDGGKQALETVQRLLPVLCQAHGL |
| | | | | TPEQVVAIASNIGGKQALETVQALLPVLCQAHGLTPQQV |
| | | | | VAIASNNGGKQALETVQRLLPVLCQAHGLTPEQVVAIAS |
| | | | | NIGGKQALETVQALLPVLCQAHGLTPQQVVAIASNNGGK |
| | | | | QALETVQRLLPVLCQAHGLTPQQVVAIASNNGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002660 | hTTR_Ex1B-L1 | NN-NG-HD-NG- | (SEQ ID NO: 91) | (SEQ ID NO: 33) |
| | | NN-NI-NN-NN- | GTCTGAGGCTGGCCC | MGDPKKKRKVIDYPYDVPDYAIDIADLRTLGYSQQQQEK |
| | | HD-NG-NN-NN- | T# | IKPKVRSTVAQHHEALVGHGFTHAHIVALSQHPAALGTV |
| | | HD-HD-HD-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNGGGKQALETVQRLLPVLCQAHGLTPEQV |
| | | | | VAIASHDGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNNGGKQALETVQRLL |
| | | | | PVLCQAHGLTPQQVVAIASNNGGKQALETVQRLLPVLCQ |
| | | | | AHGLTPEQVVAIASHDGGKQALETVQRLLPVLCQAHGLT |
| | | | | PQQVVAIASNGGGKQALETVQRLLPVLCQAHGLTPQQVV |
| | | | | AIASNNGGKQALETVQRLLPVLCQAHGLTPQQVVAIASN |
| | | | | NGGKQALETVQRLLPVLCQAHGLTPEQVVAIASHDGGKQ |
| | | | | ALETVQRLLPVLCQAHGLTPEQVVAIASHDGGKQALETV |
| | | | | QRLLPVLCQAHGLTPEQVVAIASHDGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002660 | hTTR_Ex1B-R1 | NI-NN-NN-NI- | (SEQ ID NO: 92) | (SEQ ID NO: 34) |
| | | NI-NG-NN-NN- | AGGAATGGGATGTCA | MGDPKKKRKVIDKETAAAKFERQHMDSIDIADLRTLGYS |
| | | NN-NI-NG-NN- | T# | QQQQEKIKPKVRSTVAQHHEALVGHGFTHAHIVALSQHP |
| | | NG-HD-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASNIGGKQALETVQALLPVL |
| | | | | CQAHGLTPQQVVAIASNNGGKQALETVQRLLPVLCQAHG |
| | | | | LTPQQVVAIASNNGGKQALETVQRLLPVLCQAHGLTPEQ |
| | | | | VVAIASNIGGKQALETVQALLPVLCQAHGLTPEQVVAIA |
| | | | | SNIGGKQALETVQALLPVLCQAHGLTPQQVVAIASNGGG |
| | | | | KQALETVQRLLPVLCQAHGLTPQQVVAIASNNGGKQALE |
| | | | | TVQRLLPVLCQAHGLTPQQVVAIASNNGGKQALETVQRL |
| | | | | LPVLCQAHGLTPQQVVAIASNNGGKQALETVQRLLPVLC |
| | | | | QAHGLTPEQVVAIASNIGGKQALETVQALLPVLCQAHGL |
| | | | | TPQQVVAIASNGGGKQALETVQRLLPVLCQAHGLTPQQV |
| | | | | VAIASNNGGKQALETVQRLLPVLCQAHGLTPQQVVAIAS |
| | | | | NGGGKQALETVQRLLPVLCQAHGLTPEQVVAIASHDGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002661 | hSMN2_3ssEx8A-L1 | HD-NG-NN-NN- | (SEQ ID NO: 93) | (SEQ ID NO: 35) |
| | | NG-NG-HD-NG- | CTGGTTCTAATTTCT | MGDPKKKRKVIDYPYDVPDYAIDIADLRTLGYSQQQQEK |
| | | NI-NI-NG-NG- | T# | IKPKVRSTVAQHHEALVGHGFTHAHIVALSQHPAALGTV |
| | | NG-HD-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPQQVVAIASNGGGKQALETVQRLLPVLCQAHGLTPQQV |
| | | | | VAIASNNGGKQALETVQRLLPVLCQAHGLTPQQVVAIAS |
| | | | | NNGGKQALETVQRLLPVLCQAHGLTPQQVVAIASNGGGK |
| | | | | QALETVQRLLPVLCQAHGLTPQQVVAIASNGGGKQALET |
| | | | | VQRLLPVLCQAHGLTPEQVVAIASHDGGKQALETVQRLL |
| | | | | PVLCQAHGLTPQQVVAIASNGGGKQALETVQRLLPVLCQ |
| | | | | AHGLTPEQVVAIASNIGGKQALETVQALLPVLCQAHGLT |
| | | | | PEQVVAIASNIGGKQALETVQALLPVLCQAHGLTPQQVV |
| | | | | AIASNGGGKQALETVQRLLPVLCQAHGLTPQQVVAIASN |
| | | | | GGGKQALETVQRLLPVLCQAHGLTPQQVVAIASNGGGKQ |
| | | | | ALETVQRLLPVLCQAHGLTPEQVVAIASHDGGKQALETV |
| | | | | QRLLPVLCQAHGLTPQQVVAIASNGGGKQALETVQRLLP |
| | | | | VLCQAHGLTPQQVVAIASNGGGRPALESIVAQLSRPDPS |
| | | | | GSGSGGDPISRSQLVKSELEEKKSELRHKLKYVPHEYIE |
| | | | | LIEIARNSTQDRILEMKVMEFFMKVYGYRGKHLGGSRKP |
| | | | | DGAIYTVGSPIDYGVIVDTKAYSGGYNLPIGQADEMQRY |
| | | | | VEENQTRNKHINPNEWWKVYPSSVTEFKFLFVSGHFKGN |
| | | | | YKAQLTRLNHITNCNGAVLSVEELLIGGEMIKAGTLTLE |
| | | | | EVRRKFNNGEINFAAD |
| T002661 | hSMN2_3ssEx8A-R1 | NN-HD-NG-NN- | (SEQ ID NO: 94) | (SEQ ID NO: 36) |
| | | HD-NG-HD-NG- | GCTGCTCTATGCCAG | MGDPKKKRKVIDKETAAAKFERQHMDSIDIADLRTLGYS |
| | | NI-NG-NN-HD- | T# | QQQQEKIKPKVRSTVAQHHEALVGHGFTHAHIVALSQHP |
| | | HD-NI-NN-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASHDGGKQALETVQRLLPVLCQAHG |
| | | | | LTPQQVVAIASNGGGKQALETVQRLLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPQQVVAIASNGGG |
| | | | | KQALETVQRLLPVLCQAHGLTPEQVVAIASHDGGKQALE |
| | | | | TVQRLLPVLCQAHGLTPQQVVAIASNGGGKQALETVQRL |
| | | | | LPVLCQAHGLTPEQVVAIASNIGGKQALETVQALLPVLC |
| | | | | QAHGLTPQQVVAIASNGGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | HDGGKQALETVQRLLPVLCQAHGLTPEQVVAIASNIGGK |
| | | | | QALETVQALLPVLCQAHGLTPQQVVAIASNNGGKQALET |
| | | | | VQRLLPVLCQAHGLTPQQVVAIASNGGGRPALESIVAQL |
| | | | | SRPDPSGSGSGGDPISRSQLVKSELEEKKSELRHKLKYV |
| | | | | PHEYIELIEIARNSTQDRILEMKVMEFFMKVYGYRGKHL |
| | | | | GGSRKPDGAIYTVGSPIDYGVIVDTKAYSGGYNLPIGQA |
| | | | | DEMQRYVEENQTRNKHINPNEWWKVYPSSVTEFKFLFVS |
| | | | | GHFKGNYKAQLTRLNHITNCNGAVLSVEELLIGGEMIKA |
| | | | | GTLTLEEVRRKFNNGEINFAAD |
| T002662 | hSMN2_3ssEx8B-L1 | NN-NN-NG-NG- | (SEQ ID NO: 95) | (SEQ ID NO: 37) |
| | | HD-NG-NI-NI- | GGTTCTAATTTCTCA | MGDPKKKRKVIDYPYDVPDYAIDIADLRTLGYSQQQQEK |
| | | NG-NG-NG-HD- | T# | IKPKVRSTVAQHHEALVGHGFTHAHIVALSQHPAALGTV |
| | | NG-HD-NI-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNNGGKQALETVQRLLPVLCQAHGLTPQQV |
| | | | | VAIASNGGGKQALETVQRLLPVLCQAHGLTPQQVVAIAS |
| | | | | NGGGKQALETVQRLLPVLCQAHGLTPEQVVAIASHDGGK |
| | | | | QALETVQRLLPVLCQAHGLTPQQVVAIASNGGGKQALET |
| | | | | VQRLLPVLCQAHGLTPEQVVAIASNIGGKQALETVQALL |
| | | | | PVLCQAHGLTPEQVVAIASNIGGKQALETVQALLPVLCQ |
| | | | | AHGLTPQQVVAIASNGGGKQALETVQRLLPVLCQAHGLT |
| | | | | PQQVVAIASNGGGKQALETVQRLLPVLCQAHGLTPQQVV |
| | | | | AIASNGGGKQALETVQRLLPVLCQAHGLTPEQVVAIASH |
| | | | | DGGKQALETVQRLLPVLCQAHGLTPQQVVAIASNGGGKQ |
| | | | | ALETVQRLLPVLCQAHGLTPEQVVAIASHDGGKQALETV |
| | | | | QRLLPVLCQAHGLTPEQVVAIASNIGGKQALETVQALLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002662 | hSMN2_3ssEx8B-R1 | NG-NI-NN-NG- | (SEQ ID NO: 96) | (SEQ ID NO: 38) |
| | | NN-HD-NG-NN- | TAGTGCTGCTCTATG | MGDPKKKRKVIDKETAAAKFERQHMDSIDIADLRTLGYS |
| | | HD-NG-HD-NG- | T# | QQQQEKIKPKVRSTVAQHHEALVGHGFTHAHIVALSQHP |
| | | NI-NG-NN-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNGGGKQALETVQRLLPVL |
| | | | | CQAHGLTPEQVVAIASNIGGKQALETVQALLPVLCQAHG |
| | | | | LTPQQVVAIASNNGGKQALETVQRLLPVLCQAHGLTPQQ |
| | | | | VVAIASNGGGKQALETVQRLLPVLCQAHGLTPQQVVAIA |
| | | | | SNNGGKQALETVQRLLPVLCQAHGLTPEQVVAIASHDGG |
| | | | | KQALETVQRLLPVLCQAHGLTPQQVVAIASNGGGKQALE |
| | | | | TVQRLLPVLCQAHGLTPQQVVAIASNNGGKQALETVQRL |
| | | | | LPVLCQAHGLTPEQVVAIASHDGGKQALETVQRLLPVLC |
| | | | | QAHGLTPQQVVAIASNGGGKQALETVQRLLPVLCQAHGL |
| | | | | TPEQVVAIASHDGGKQALETVQRLLPVLCQAHGLTPQQV |
| | | | | VAIASNGGGKQALETVQRLLPVLCQAHGLTPEQVVAIAS |
| | | | | NIGGKQALETVQALLPVLCQAHGLTPQQVVAIASNGGGK |
| | | | | QALETVQRLLPVLCQAHGLTPQQVVAIASNNGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002663 | hSMN2_ISS100A-L1 | NG-HD-NI-NN- | (SEQ ID NO: 97) | (SEQ ID NO: 39) |
| | | NI-NG-NN-NG- | TCAGATGTTAGAAAG | MGDPKKKRKVIDYPYDVPDYAIDIADLRTLGYSQQQQEK |
| | | NG-NI-NN-NI- | T# | IKPKVRSTVAQHHEALVGHGFTHAHIVALSQHPAALGTV |
| | | NI-NI-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPEQVVAIASHDGGKQALETVQRLLPVLCQAHGLTPEQV |
| | | | | VAIASNIGGKQALETVQALLPVLCQAHGLTPQQVVAIAS |
| | | | | NNGGKQALETVQRLLPVLCQAHGLTPEQVVAIASNIGGK |
| | | | | QALETVQALLPVLCQAHGLTPQQVVAIASNGGGKQALET |
| | | | | VQRLLPVLCQAHGLTPQQVVAIASNNGGKQALETVQRLL |
| | | | | PVLCQAHGLTPQQVVAIASNGGGKQALETVQRLLPVLCQ |
| | | | | AHGLTPQQVVAIASNGGGKQALETVQRLLPVLCQAHGLT |
| | | | | PEQVVAIASNIGGKQALETVQALLPVLCQAHGLTPQQVV |
| | | | | AIASNNGGKQALETVQRLLPVLCQAHGLTPEQVVAIASN |
| | | | | IGGKQALETVQALLPVLCQAHGLTPEQVVAIASNIGGKQ |
| | | | | ALETVQALLPVLCQAHGLTPEQVVAIASNIGGKQALETV |
| | | | | QALLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002663 | hSMN2_ISS100A-R1 | NG-NG-NI-NI- | (SEQ ID NO: 98) | (SEQ ID NO: 40) |
| | | NG-NI-NG-NG- | TTAATATTGATTGTT | MGDPKKKRKVIDKETAAAKFERQHMDSIDIADLRTLGYS |
| | | NN-NI-NG-NG- | T# | QQQQEKIKPKVRSTVAQHHEALVGHGFTHAHIVALSQHP |
| | | NN-NG-NG-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNGGGKQALETVQRLLPVL |
| | | | | CQAHGLTPQQVVAIASNGGGKQALETVQRLLPVLCQAHG |
| | | | | LTPEQVVAIASNIGGKQALETVQALLPVLCQAHGLTPEQ |
| | | | | VVAIASNIGGKQALETVQALLPVLCQAHGLTPQQVVAIA |
| | | | | SNGGGKQALETVQRLLPVLCQAHGLTPEQVVAIASNIGG |
| | | | | KQALETVQALLPVLCQAHGLTPQQVVAIASNGGGKQALE |
| | | | | TVQRLLPVLCQAHGLTPQQVVAIASNGGGKQALETVQRL |
| | | | | LPVLCQAHGLTPQQVVAIASNNGGKQALETVQRLLPVLC |
| | | | | QAHGLTPEQVVAIASNIGGKQALETVQALLPVLCQAHGL |
| | | | | TPQQVVAIASNGGGKQALETVQRLLPVLCQAHGLTPQQV |
| | | | | VAIASNGGGKQALETVQRLLPVLCQAHGLTPQQVVAIAS |
| | | | | NNGGKQALETVQRLLPVLCQAHGLTPQQVVAIASNGGGK |
| | | | | QALETVQRLLPVLCQAHGLTPQQVVAIASNGGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002664 | hSMN2_ISS-NIA-L1 | NG-NI-NI-NN- | (SEQ ID NO: 99) | (SEQ ID NO: 41) |
| | | NN-NI-NN-NG- | TAAGGAGTAAGTCTG | MGDPKKKRKVIDYPYDVPDYAIDIADLRTLGYSQQQQEK |
| | | NI-NI-NN-NG- | T# | IKPKVRSTVAQHHEALVGHGFTHAHIVALSQHPAALGTV |
| | | HD-NG-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPEQVVAIASNIGGKQALETVQALLPVLCQAHGLTPEQV |
| | | | | VAIASNIGGKQALETVQALLPVLCQAHGLTPQQVVAIAS |
| | | | | NNGGKQALETVQRLLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNNGGKQALETVQRLL |
| | | | | PVLCQAHGLTPQQVVAIASNGGGKQALETVQRLLPVLCQ |
| | | | | AHGLTPEQVVAIASNIGGKQALETVQALLPVLCQAHGLT |
| | | | | PEQVVAIASNIGGKQALETVQALLPVLCQAHGLTPQQVV |
| | | | | AIASNNGGKQALETVQRLLPVLCQAHGLTPQQVVAIASN |
| | | | | GGGKQALETVQRLLPVLCQAHGLTPEQVVAIASHDGGKQ |
| | | | | ALETVQRLLPVLCQAHGLTPQQVVAIASNGGGKQALETV |
| | | | | QRLLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002664 | hSMN2_ISS-NIA-R1 | NG-NI-HD-NI- | (SEQ ID NO: 100) | (SEQ ID NO: 42) |
| | | NI-NI-NI-NN- | TACAAAAGTAAGATT | MGDPKKKRKVIDKETAAAKFERQHMDSIDIADLRTLGYS |
| | | NG-NI-NI-NN- | T# | QQQQEKIKPKVRSTVAQHHEALVGHGFTHAHIVALSQHP |
| | | NI-NG-NG-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNGGGKQALETVQRLLPVL |
| | | | | CQAHGLTPEQVVAIASNIGGKQALETVQALLPVLCQAHG |
| | | | | LTPEQVVAIASHDGGKQALETVQRLLPVLCQAHGLTPEQ |
| | | | | VVAIASNIGGKQALETVQALLPVLCQAHGLTPEQVVAIA |
| | | | | SNIGGKQALETVQALLPVLCQAHGLTPEQVVAIASNIGG |
| | | | | KQALETVQALLPVLCQAHGLTPEQVVAIASNIGGKQALE |
| | | | | TVQALLPVLCQAHGLTPQQVVAIASNNGGKQALETVQRL |
| | | | | LPVLCQAHGLTPQQVVAIASNGGGKQALETVQRLLPVLC |
| | | | | QAHGLTPEQVVAIASNIGGKQALETVQALLPVLCQAHGL |
| | | | | TPEQVVAIASNIGGKQALETVQALLPVLCQAHGLTPQQV |
| | | | | VAIASNNGGKQALETVQRLLPVLCQAHGLTPEQVVAIAS |
| | | | | NIGGKQALETVQALLPVLCQAHGLTPQQVVAIASNGGGK |
| | | | | QALETVQRLLPVLCQAHGLTPQQVVAIASNGGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002665 | hIDOL_Ex2A-L1 | NG-NG-NI-NG- | (SEQ ID NO: 101) | (SEQ ID NO: 43) |
| | | NN-NN-HD-NG- | TTATGGCTAAACCTG | MGDPKKKRKVIDYPYDVPDYAIDIADLRTLGYSQQQQEK |
| | | NI-NI-NI-HD- | T# | IKPKVRSTVAQHHEALVGHGFTHAHIVALSQHPAALGTV |
| | | HD-NG-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPQQVVAIASNGGGKQALETVQRLLPVLCQAHGLTPEQV |
| | | | | VAIASNIGGKQALETVQALLPVLCQAHGLTPQQVVAIAS |
| | | | | NGGGKQALETVQRLLPVLCQAHGLTPQQVVAIASNNGGK |
| | | | | QALETVQRLLPVLCQAHGLTPQQVVAIASNNGGKQALET |
| | | | | VQRLLPVLCQAHGLTPEQVVAIASHDGGKQALETVQRLL |
| | | | | PVLCQAHGLTPQQVVAIASNGGGKQALETVQRLLPVLCQ |
| | | | | AHGLTPEQVVAIASNIGGKQALETVQALLPVLCQAHGLT |
| | | | | PEQVVAIASNIGGKQALETVQALLPVLCQAHGLTPEQVV |
| | | | | AIASNIGGKQALETVQALLPVLCQAHGLTPEQVVAIASH |
| | | | | DGGKQALETVQRLLPVLCQAHGLTPEQVVAIASHDGGKQ |
| | | | | ALETVQRLLPVLCQAHGLTPQQVVAIASNGGGKQALETV |
| | | | | QRLLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002665 | hIDOL_Ex2A-R1 | NI-NN-HD-HD- | (SEQ ID NO: 102) | (SEQ ID NO: 44) |
| | | HD-NI-NG-HD- | AGCCCATCCATCTGC | MGDPKKKRKVIDKETAAAKFERQHMDSIDIADLRTLGYS |
| | | HD-NI-NG-HD- | T# | QQQQEKIKPKVRSTVAQHHEALVGHGFTHAHIVALSQHP |
| | | NG-NN-HD-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASNIGGKQALETVQALLPVL |
| | | | | CQAHGLTPQQVVAIASNNGGKQALETVQRLLPVLCQAHG |
| | | | | LTPEQVVAIASHDGGKQALETVQRLLPVLCQAHGLTPEQ |
| | | | | VVAIASHDGGKQALETVQRLLPVLCQAHGLTPEQVVAIA |
| | | | | SHDGGKQALETVQRLLPVLCQAHGLTPEQVVAIASNIGG |
| | | | | KQALETVQALLPVLCQAHGLTPQQVVAIASNGGGKQALE |
| | | | | TVQRLLPVLCQAHGLTPEQVVAIASHDGGKQALETVQRL |
| | | | | LPVLCQAHGLTPEQVVAIASHDGGKQALETVQRLLPVLC |
| | | | | QAHGLTPEQVVAIASNIGGKQALETVQALLPVLCQAHGL |
| | | | | TPQQVVAIASNGGGKQALETVQRLLPVLCQAHGLTPEQV |
| | | | | VAIASHDGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASHDGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002666 | hIDOL_Ex3A-L1 | NN-HD-HD-NI- | (SEQ ID NO: 103) | (SEQ ID NO: 45) |
| | | NI-NN-NN-NI- | GCCAAGGAGCTCTCC | MGDPKKKRKVIDYPYDVPDYAIDIADLRTLGYSQQQQEK |
| | | NN-HD-NG-HD- | T# | IKPKVRSTVAQHHEALVGHGFTHAHIVALSQHPAALGTV |
| | | NG-HD-HD-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPEQVVAIASHDGGKQALETVQRLLPVLCQAHGLTPEQV |
| | | | | VAIASHDGGKQALETVQRLLPVLCQAHGLTPEQVVAIAS |
| | | | | NIGGKQALETVQALLPVLCQAHGLTPEQVVAIASNIGGK |
| | | | | QALETVQALLPVLCQAHGLTPQQVVAIASNNGGKQALET |
| | | | | VQRLLPVLCQAHGLTPQQVVAIASNNGGKQALETVQRLL |
| | | | | PVLCQAHGLTPEQVVAIASNIGGKQALETVQALLPVLCQ |
| | | | | AHGLTPQQVVAIASNNGGKQALETVQRLLPVLCQAHGLT |
| | | | | PEQVVAIASHDGGKQALETVQRLLPVLCQAHGLTPQQVV |
| | | | | AIASNGGGKQALETVQRLLPVLCQAHGLTPEQVVAIASH |
| | | | | DGGKQALETVQRLLPVLCQAHGLTPQQVVAIASNGGGKQ |
| | | | | ALETVQRLLPVLCQAHGLTPEQVVAIASHDGGKQALETV |
| | | | | QRLLPVLCQAHGLTPEQVVAIASHDGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002666 | hIDOL_Ex3A-R1 | NI-NG-NG-HD- | (SEQ ID NO: 104) | (SEQ ID NO: 46) |
| | | NI-NI-HD-NI- | ATTCAACAGCCTCAC | MGDPKKKRKVIDKETAAAKFERQHMDSIDIADLRTLGYS |
| | | NN-HD-HD-NG- | T# | QQQQEKIKPKVRSTVAQHHEALVGHGFTHAHIVALSQHP |
| | | HD-NI-HD-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASNIGGKQALETVQALLPVL |
| | | | | CQAHGLTPQQVVAIASNGGGKQALETVQRLLPVLCQAHG |
| | | | | LTPQQVVAIASNGGGKQALETVQRLLPVLCQAHGLTPEQ |
| | | | | VVAIASHDGGKQALETVQRLLPVLCQAHGLTPEQVVAIA |
| | | | | SNIGGKQALETVQALLPVLCQAHGLTPEQVVAIASNIGG |
| | | | | KQALETVQALLPVLCQAHGLTPEQVVAIASHDGGKQALE |
| | | | | TVQRLLPVLCQAHGLTPEQVVAIASNIGGKQALETVQAL |
| | | | | LPVLCQAHGLTPQQVVAIASNNGGKQALETVQRLLPVLC |
| | | | | QAHGLTPEQVVAIASHDGGKQALETVQRLLPVLCQAHGL |
| | | | | TPEQVVAIASHDGGKQALETVQRLLPVLCQAHGLTPQQV |
| | | | | VAIASNGGGKQALETVQRLLPVLCQAHGLTPEQVVAIAS |
| | | | | HDGGKQALETVQRLLPVLCQAHGLTPEQVVAIASNIGGK |
| | | | | QALETVQALLPVLCQAHGLTPEQVVAIASHDGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002667 | hANGPTL3_Ex1A-L1 | NI-NG-NG-NN- | (SEQ ID NO: 105) | (SEQ ID NO: 47) |
| | | NG-NG-HD-HD- | ATTGTTCCTCTAGTT | MGDPKKKRKVIDYPYDVPDYAIDIADLRTLGYSQQQQEK |
| | | NG-HD-NG-NI- | T# | IKPKVRSTVAQHHEALVGHGFTHAHIVALSQHPAALGTV |
| | | NN-NG-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASNIGGKQALETVQALLPVLCQAHGL |
| | | | | TPQQVVAIASNGGGKQALETVQRLLPVLCQAHGLTPQQV |
| | | | | VAIASNGGGKQALETVQRLLPVLCQAHGLTPQQVVAIAS |
| | | | | NNGGKQALETVQRLLPVLCQAHGLTPQQVVAIASNGGGK |
| | | | | QALETVQRLLPVLCQAHGLTPQQVVAIASNGGGKQALET |
| | | | | VQRLLPVLCQAHGLTPEQVVAIASHDGGKQALETVQRLL |
| | | | | PVLCQAHGLTPEQVVAIASHDGGKQALETVQRLLPVLCQ |
| | | | | AHGLTPQQVVAIASNGGGKQALETVQRLLPVLCQAHGLT |
| | | | | PEQVVAIASHDGGKQALETVQRLLPVLCQAHGLTPQQVV |
| | | | | AIASNGGGKQALETVQRLLPVLCQAHGLTPEQVVAIASN |
| | | | | IGGKQALETVQALLPVLCQAHGLTPQQVVAIASNNGGKQ |
| | | | | ALETVQRLLPVLCQAHGLTPQQVVAIASNGGGKQALETV |
| | | | | QRLLPVLCQAHGLTPQQVVAIASNGGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002667 | hANGPTL3_Ex1A-R1 | NN-NI-NG-NN- | (SEQ ID NO: 106) | (SEQ ID NO: 48) |
| | | NI-NI-NG-NG- | GATGAATTGTCTTGA | MGDPKKKRKVIDKETAAAKFERQHMDSIDIADLRTLGYS |
| | | NN-NG-HD-NG- | T# | QQQQEKIKPKVRSTVAQHHEALVGHGFTHAHIVALSQHP |
| | | NG-NN-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASNIGGKQALETVQALLPVLCQAHG |
| | | | | LTPQQVVAIASNGGGKQALETVQRLLPVLCQAHGLTPQQ |
| | | | | VVAIASNNGGKQALETVQRLLPVLCQAHGLTPEQVVAIA |
| | | | | SNIGGKQALETVQALLPVLCQAHGLTPEQVVAIASNIGG |
| | | | | KQALETVQALLPVLCQAHGLTPQQVVAIASNGGGKQALE |
| | | | | TVQRLLPVLCQAHGLTPQQVVAIASNGGGKQALETVQRL |
| | | | | LPVLCQAHGLTPQQVVAIASNNGGKQALETVQRLLPVLC |
| | | | | QAHGLTPQQVVAIASNGGGKQALETVQRLLPVLCQAHGL |
| | | | | TPEQVVAIASHDGGKQALETVQRLLPVLCQAHGLTPQQV |
| | | | | VAIASNGGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002668 | hANGPTL3_Ex2A-L1 | HD-HD-NI-NN- | (SEQ ID NO: 107) | (SEQ ID NO: 49) |
| | | NI-HD-NG-NG- | CCAGACTTTTGTAGA | MGDPKKKRKVIDYPYDVPDYAIDIADLRTLGYSQQQQEK |
| | | NG-NG-NN-NG- | T# | IKPKVRSTVAQHHEALVGHGFTHAHIVALSQHPAALGTV |
| | | NI-NN-NI-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPEQVVAIASHDGGKQALETVQRLLPVLCQAHGL |
| | | | | TPEQVVAIASHDGGKQALETVQRLLPVLCQAHGLTPEQV |
| | | | | VAIASNIGGKQALETVQALLPVLCQAHGLTPQQVVAIAS |
| | | | | NNGGKQALETVQRLLPVLCQAHGLTPEQVVAIASNIGGK |
| | | | | QALETVQALLPVLCQAHGLTPEQVVAIASHDGGKQALET |
| | | | | VQRLLPVLCQAHGLTPQQVVAIASNGGGKQALETVQRLL |
| | | | | PVLCQAHGLTPQQVVAIASNGGGKQALETVQRLLPVLCQ |
| | | | | AHGLTPQQVVAIASNGGGKQALETVQRLLPVLCQAHGLT |
| | | | | PQQVVAIASNGGGKQALETVQRLLPVLCQAHGLTPQQVV |
| | | | | AIASNNGGKQALETVQRLLPVLCQAHGLTPQQVVAIASN |
| | | | | GGGKQALETVQRLLPVLCQAHGLTPEQVVAIASNIGGKQ |
| | | | | ALETVQALLPVLCQAHGLTPQQVVAIASNNGGKQALETV |
| | | | | QRLLPVLCQAHGLTPEQVVAIASNIGGKQALETVQALLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002668 | hANGPTL3_Ex2A-R1 | NN-NN-NI-NN- | (SEQ ID NO: 108) | (SEQ ID NO: 50) |
| | | NI-NI-NN-NN- | GGAGAAGGTCTTTGA | MGDPKKKRKVIDKETAAAKFERQHMDSIDIADLRTLGYS |
| | | NG-HD-NG-NG- | T# | QQQQEKIKPKVRSTVAQHHEALVGHGFTHAHIVALSQHP |
| | | NG-NN-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPQQVVAIASNNGGKQALETVQRLLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SNIGGKQALETVQALLPVLCQAHGLTPEQVVAIASNIGG |
| | | | | KQALETVQALLPVLCQAHGLTPQQVVAIASNNGGKQALE |
| | | | | TVQRLLPVLCQAHGLTPQQVVAIASNNGGKQALETVQRL |
| | | | | LPVLCQAHGLTPQQVVAIASNGGGKQALETVQRLLPVLC |
| | | | | QAHGLTPEQVVAIASHDGGKQALETVQRLLPVLCQAHGL |
| | | | | TPQQVVAIASNGGGKQALETVQRLLPVLCQAHGLTPQQV |
| | | | | VAIASNGGGKQALETVQRLLPVLCQAHGLTPQQVVAIAS |
| | | | | NGGGKQALETVQRLLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002669 | hPCSK9_Ex3A-L1 | NG-NN-HD-HD- | (SEQ ID NO: 109) | (SEQ ID NO: 51) |
| | | HD-HD-NI-NG- | TGCCCCATGTCGACT | MGDPKKKRKVIDYPYDVPDYAIDIADLRTLGYSQQQQEK |
| | | NN-NG-HD-NN- | T# | IKPKVRSTVAQHHEALVGHGFTHAHIVALSQHPAALGTV |
| | | NI-HD-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | HDGGKQALETVQRLLPVLCQAHGLTPEQVVAIASHDGGK |
| | | | | QALETVQRLLPVLCQAHGLTPEQVVAIASHDGGKQALET |
| | | | | VQRLLPVLCQAHGLTPEQVVAIASNIGGKQALETVQALL |
| | | | | PVLCQAHGLTPQQVVAIASNGGGKQALETVQRLLPVLCQ |
| | | | | AHGLTPQQVVAIASNNGGKQALETVQRLLPVLCQAHGLT |
| | | | | PQQVVAIASNGGGKQALETVQRLLPVLCQAHGLTPEQVV |
| | | | | AIASHDGGKQALETVQRLLPVLCQAHGLTPQQVVAIASN |
| | | | | NGGKQALETVQRLLPVLCQAHGLTPEQVVAIASNIGGKQ |
| | | | | ALETVQALLPVLCQAHGLTPEQVVAIASHDGGKQALETV |
| | | | | QRLLPVLCQAHGLTPQQVVAIASNGGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002669 | hPCSK9_Ex3A-R1 | HD-NG-NN-NN- | (SEQ ID NO: 110) | (SEQ ID NO: 52) |
| | | NN-HD-NI-NI- | CTGGGCAAAGACAGA | MGDPKKKRKVIDKETAAAKFERQHMDSIDIADLRTLGYS |
| | | NI-NN-NI-HD- | T# | QQQQEKIKPKVRSTVAQHHEALVGHGFTHAHIVALSQHP |
| | | NI-NN-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASHDGGKQALETVQRLLPVL |
| | | | | CQAHGLTPQQVVAIASNGGGKQALETVQRLLPVLCQAHG |
| | | | | LTPQQVVAIASNNGGKQALETVQRLLPVLCQAHGLTPQQ |
| | | | | VVAIASNNGGKQALETVQRLLPVLCQAHGLTPQQVVAIA |
| | | | | SNNGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPEQVVAIASNIGGKQALETVQAL |
| | | | | LPVLCQAHGLTPEQVVAIASNIGGKQALETVQALLPVLC |
| | | | | QAHGLTPQQVVAIASNNGGKQALETVQRLLPVLCQAHGL |
| | | | | TPEQVVAIASNIGGKQALETVQALLPVLCQAHGLTPEQV |
| | | | | VAIASHDGGKQALETVQRLLPVLCQAHGLTPEQVVAIAS |
| | | | | NIGGKQALETVQALLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002670 | hPCSK9_Ex12A-L1 | NN-NN-HD-NI- | (SEQ ID NO: 111) | (SEQ ID NO: 53) |
| | | NN-NN-NG-NN- | GGCAGGTGACCGTGG | MGDPKKKRKVIDYPYDVPDYAIDIADLRTLGYSQQQQEK |
| | | NI-HD-HD-NN- | T# | IKPKVRSTVAQHHEALVGHGFTHAHIVALSQHPAALGTV |
| | | NG-NN-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | NIGGKQALETVQALLPVLCQAHGLTPQQVVAIASNNGGK |
| | | | | QALETVQRLLPVLCQAHGLTPQQVVAIASNNGGKQALET |
| | | | | VQRLLPVLCQAHGLTPQQVVAIASNGGGKQALETVQRLL |
| | | | | PVLCQAHGLTPQQVVAIASNNGGKQALETVQRLLPVLCQ |
| | | | | AHGLTPEQVVAIASNIGGKQALETVQALLPVLCQAHGLT |
| | | | | PEQVVAIASHDGGKQALETVQRLLPVLCQAHGLTPEQVV |
| | | | | AIASHDGGKQALETVQRLLPVLCQAHGLTPQQVVAIASN |
| | | | | NGGKQALETVQRLLPVLCQAHGLTPQQVVAIASNGGGKQ |
| | | | | ALETVQRLLPVLCQAHGLTPQQVVAIASNNGGKQALETV |
| | | | | QRLLPVLCQAHGLTPQQVVAIASNNGGKQALETVQRLLP |
| | | | | VLCQAHGLTPQQVVAIASNGGGRPALESIVAQLSRPDPA |
| | | | | LAALTNDHLVALACLGGRPALDAVKKGLGDPISRSQLVK |
| | | | | SELEEKKSELRHKLKYVPHEYIELIEIARNSTQDRILEM |
| | | | | KVMEFFMKVYGYRGKHLGGSRKPDGAIYTVGSPIDYGVI |
| | | | | VDTKAYSGGYNLPIGQADEMQRYVEENQTRNKHINPNEW |
| | | | | WKVYPSSVTEFKFLFVSGHFKGNYKAQLTRLNHITNCNG |
| | | | | AVLSVEELLIGGEMIKAGTLTLEEVRRKFNNGEINFAAD |
| T002670 | hPCSK9_Ex12A-R1 | NN-HD-NI-NN- | (SEQ ID NO: 112) | (SEQ ID NO: 54) |
| | | HD-HD-NI-NN- | GCAGCCAGTCAGGGT | MGDPKKKRKVIDKETAAAKFERQHMDSIDIADLRTLGYS |
| | | NG-HD-NI-NN- | T# | QQQQEKIKPKVRSTVAQHHEALVGHGFTHAHIVALSQHP |
| | | NN-NN-NG-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPQQVVAIASNNGGKQALETVQRL |
| | | | | LPVLCQAHGLTPQQVVAIASNGGGKQALETVQRLLPVLC |
| | | | | QAHGLTPEQVVAIASHDGGKQALETVQRLLPVLCQAHGL |
| | | | | TPEQVVAIASNIGGKQALETVQALLPVLCQAHGLTPQQV |
| | | | | VAIASNNGGKQALETVQRLLPVLCQAHGLTPQQVVAIAS |
| | | | | NNGGKQALETVQRLLPVLCQAHGLTPQQVVAIASNNGGK |
| | | | | QALETVQRLLPVLCQAHGLTPQQVVAIASNGGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002672 | hPCSK9_Ex3_AN-L1 | NG-NN-HD-HD- | (SEQ ID NO: 113) | (SEQ ID NO: 55) |
| | | HD-HD-NI-NG- | TGCCCCATGT5GACT | MGDPKKKRKVIDYPYDVPDYAIDIADLRTLGYSQQQQEK |
| | | NN-NG-N-NN- | T# | IKPKVRSTVAQHHEALVGHGFTHAHIVALSQHPAALGTV |
| | | NI-HD-NG-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNGGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | HDGGKQALETVQRLLPVLCQAHGLTPEQVVAIASHDGGK |
| | | | | QALETVQRLLPVLCQAHGLTPEQVVAIASHDGGKQALET |
| | | | | VQRLLPVLCQAHGLTPEQVVAIASNIGGKQALETVQALL |
| | | | | PVLCQAHGLTPQQVVAIASNGGGKQALETVQRLLPVLCQ |
| | | | | AHGLTPQQVVAIASNNGGKQALETVQRLLPVLCQAHGLT |
| | | | | PQQVVAIASNGGGKQALETVQRLLPVLCQAHGLTPQQVV |
| | | | | AIASNGGKQALETVQRLLPVLCQAHGLTPQQVVAIASNN |
| | | | | GGKQALETVQRLLPVLCQAHGLTPEQVVAIASNIGGKQA |
| | | | | LETVQALLPVLCQAHGLTPEQVVAIASHDGGKQALETVQ |
| | | | | RLLPVLCQAHGLTPQQVVAIASNGGGKQALETVQRLLPV |
| | | | | LCQAHGLTPQQVVAIASNGGGRPALESIVAQLSRPDPAL |
| | | | | AALTNDHLVALACLGGRPALDAVKKGLGDPISRSQLVKS |
| | | | | ELEEKKSELRHKLKYVPHEYIELIEIARNSTQDRILEMK |
| | | | | VMEFFMKVYGYRGKHLGGSRKPDGAIYTVGSPIDYGVIV |
| | | | | DTKAYSGGYNLPIGQADEMQRYVEENQTRNKHINPNEWW |
| | | | | KVYPSSVTEFKFLFVSGHFKGNYKAQLTRLNHITNCNGA |
| | | | | VLSVEELLIGGEMIKAGTLTLEEVRRKFNNGEINFAAD |
| T002672 | hPCSK9_Ex3A-R1 | HD-NG-NN-NN- | (SEQ ID NO: 114) | (SEQ ID NO: 56) |
| | | NN-HD-NI-NI- | CTGGGCAAAGACAGA | MGDPKKKRKVIDKETAAAKFERQHMDSIDIADLRTLGYS |
| | | NI-NN-NI-HD- | T# | QQQQEKIKPKVRSTVAQHHEALVGHGFTHAHIVALSQHP |
| | | NI-NN-NI-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPEQVVAIASHDGGKQALETVQRLLPVL |
| | | | | CQAHGLTPQQVVAIASNGGGKQALETVQRLLPVLCQAHG |
| | | | | LTPQQVVAIASNNGGKQALETVQRLLPVLCQAHGLTPQQ |
| | | | | VVAIASNNGGKQALETVQRLLPVLCQAHGLTPQQVVAIA |
| | | | | SNNGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPEQVVAIASNIGGKQALETVQAL |
| | | | | LPVLCQAHGLTPEQVVAIASNIGGKQALETVQALLPVLC |
| | | | | QAHGLTPQQVVAIASNNGGKQALETVQRLLPVLCQAHGL |
| | | | | TPEQVVAIASNIGGKQALETVQALLPVLCQAHGLTPEQV |
| | | | | VAIASHDGGKQALETVQRLLPVLCQAHGLTPEQVVAIAS |
| | | | | NIGGKQALETVQALLPVLCQAHGLTPQQVVAIASNNGGK |
| | | | | QALETVQRLLPVLCQAHGLTPEQVVAIASNIGGKQALET |
| | | | | VQALLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002673 | hPCSK9_Ex12A-R1 | NN-HD-NI-NN- | (SEQ ID NO: 115) | (SEQ ID NO: 57) |
| | | HD-HD-NI-NN- | GCAGCCAGTCAGGGT | MGDPKKKRKVIDKETAAAKFERQHMDSIDIADLRTLGYS |
| | | NG-HD-NI-NN- | T# | QQQQEKIKPKVRSTVAQHHEALVGHGFTHAHIVALSQHP |
| | | NN-NN-NG-NG# | | AALGTVAVKYQDMIAALPEATHEAIVGVGKQWSGARALE |
| | | | | ALLTVAGELRGPPLQLDTGQLLKIAKRGGVTAVEAVHAW |
| | | | | RNALTGAPLNLTPQQVVAIASNNGGKQALETVQRLLPVL |
| | | | | CQAHGLTPEQVVAIASHDGGKQALETVQRLLPVLCQAHG |
| | | | | LTPEQVVAIASNIGGKQALETVQALLPVLCQAHGLTPQQ |
| | | | | VVAIASNNGGKQALETVQRLLPVLCQAHGLTPEQVVAIA |
| | | | | SHDGGKQALETVQRLLPVLCQAHGLTPEQVVAIASHDGG |
| | | | | KQALETVQRLLPVLCQAHGLTPEQVVAIASNIGGKQALE |
| | | | | TVQALLPVLCQAHGLTPQQVVAIASNNGGKQALETVQRL |
| | | | | LPVLCQAHGLTPQQVVAIASNGGGKQALETVQRLLPVLC |
| | | | | QAHGLTPEQVVAIASHDGGKQALETVQRLLPVLCQAHGL |
| | | | | TPEQVVAIASNIGGKQALETVQALLPVLCQAHGLTPQQV |
| | | | | VAIASNNGGKQALETVQRLLPVLCQAHGLTPQQVVAIAS |
| | | | | NNGGKQALETVQRLLPVLCQAHGLTPQQVVAIASNNGGK |
| | | | | QALETVQRLLPVLCQAHGLTPQQVVAIASNGGGKQALET |
| | | | | VQRLLPVLCQAHGLTPQQVVAIASNGGGRPALESIVAQL |
| | | | | SRPDPALAALTNDHLVALACLGGRPALDAVKKGLGDPIS |
| | | | | RSQLVKSELEEKKSELRHKLKYVPHEYIELIEIARNSTQ |
| | | | | DRILEMKVMEFFMKVYGYRGKHLGGSRKPDGAIYTVGSP |
| | | | | IDYGVIVDTKAYSGGYNLPIGQADEMQRYVEENQTRNKH |
| | | | | INPNEWWKVYPSSVTEFKFLFVSGHFKGNYKAQLTRLNH |
| | | | | ITNCNGAVLSVEELLIGGEMIKAGTLTLEEVRRKFNNGE |
| | | | | INFAAD |
| T002673 | hPCSK9Ex12_AN-L1 | NN-NN-HD-NI- | (SEQ ID NO: 116) | (SEQ ID NO: 58) |
| | | NN-NN-NG-NN- | GGCAGGTGAC5GTGG | MGDPKKKRKVIDYPYDVPDYAIDIADLRTLGYSQQQQEK |
| | | NI-HD-N-NN- | T# | IKPKVRSTVAQHHEALVGHGFTHAHIVALSQHPAALGTV |
| | | NG-NN-NN-NG# | | AVKYQDMIAALPEATHEAIVGVGKQWSGARALEALLTVA |
| | | | | GELRGPPLQLDTGQLLKIAKRGGVTAVEAVHAWRNALTG |
| | | | | APLNLTPQQVVAIASNNGGKQALETVQRLLPVLCQAHGL |
| | | | | TPQQVVAIASNNGGKQALETVQRLLPVLCQAHGLTPEQV |
| | | | | VAIASHDGGKQALETVQRLLPVLCQAHGLTPEQVVAIAS |
| | | | | NIGGKQALETVQALLPVLCQAHGLTPQQVVAIASNNGGK |
| | | | | QALETVQRLLPVLCQAHGLTPQQVVAIASNNGGKQALET |
| | | | | VQRLLPVLCQAHGLTPQQVVAIASNGGGKQALETVQRLL |
| | | | | PVLCQAHGLTPQQVVAIASNNGGKQALETVQRLLPVLCQ |
| | | | | AHGLTPEQVVAIASNIGGKQALETVQALLPVLCQAHGLT |
| | | | | PEQVVAIASHDGGKQALETVQRLLPVLCQAHGLTPQQVV |
| | | | | AIASNGGKQALETVQRLLPVLCQAHGLTPQQVVAIASNN |
| | | | | GGKQALETVQRLLPVLCQAHGLTPQQVVAIASNGGGKQA |
| | | | | LETVQRLLPVLCQAHGLTPQQVVAIASNNGGKQALETVQ |
| | | | | RLLPVLCQAHGLTPQQVVAIASNNGGKQALETVQRLLPV |
| | | | | LCQAHGLTPQQVVAIASNGGGRPALESIVAQLSRPDPAL |
| | | | | AALTNDHLVALACLGGRPALDAVKKGLGDPISRSQLVKS |
| | | | | ELEEKKSELRHKLKYVPHEYIELIEIARNSTQDRILEMK |
| | | | | VMEFFMKVYGYRGKHLGGSRKPDGAIYTVGSPIDYGVIV |
| | | | | DTKAYSGGYNLPIGQADEMQRYVEENQTRNKHINPNEWW |
| | | | | KVYPSSVTEFKFLFVSGHFKGNYKAQLTRLNHITNCNGA |
| | | | | VLSVEELLIGGEMIKAGTLTLEEVRRKFNNGEINFAAD |

1) A therapeutic composition comprising (1) an endonuclease reagent engineered to target the cccDNA of HBV in-vivo which binds one target sequence selected from SEQ ID NO: 1 to 20 and (2) an antiviral compound, said antiviral compound displaying none endonuclease activity.

2) A therapeutic composition according to claim 1, wherein said endonuclease reagent is a TALE-nuclease monomer.

3) A therapeutic composition according to claim 1, wherein said antiviral compound is selected from:
Inhibitor of sodium taurocholate cotransporting polypeptide (NTCP), such as Myrcludex;
cccDNA inhibitor, such as disubstituted sulfonamide (DSS) compounds, antibodies inducing Lymphotoxin beta receptor activation or LTβR agonists;
RNAi or compounds aiming at reducing HBV genes expression, in particular Helioxanthin or Ethanol extract from Ampelopsis sinica root;
La protein inhibitor, such as HBSC11;
Capsid allosteric modulators, such as ABI H0731, JNJ 56136379 (or JNJ379), Morphothiadine (GLS4), NVR 3 778 or NVR1221;
Reverse transcriptase inhibitors, in particular nucleoside analogs, such as Lamivudine, Telbivudine, Entecavir, Adefovir, Tenofovir, Besifovir, MIV-210, MCC-478 or Alamifovir;
Inhibition of HBsAg release, such as REP2139 and GC1102;
Immunoregulators, such as Thymosin-al;
Vesatolimod (GS-9620);
Vaccines, such as GS-4774, ABX-203, TG-1050, INO-1800; FP-02.2; and
Immune stimulators, such as SB-9200, AIC649, Cellular inhibitor of apoptosis proteins (cIAPs) Cytokines: IL-21 and/or recombinant human IL-7 and antibodies antagonist of Immune checkpoint (Nivolumab and Pembrolizumab).

4) A therapeutic composition according to any one of claims 1 to 3, wherein said endonuclease reagent is encapsulated by a method comprising the steps of:
a) Engineering a endonuclease reagent under RNA form;
b) Complexing said endonuclease reagent with at least one biodegradable matrix comprising at least a core hydrophobic domain and a proximal polar domain to favor interactions with water molecules;
c) Forming particles encapsulating said endonuclease reagent of 50 to 100 nm diameter range.

5) A therapeutic composition according to claim 4, wherein said RNA encodes an endonuclease reagent, which is a RNA-guided endonuclease.

6) A therapeutic composition according to claim 5, wherein said RNA-guided endonuclease is cas9 or Cpf1.

7) A therapeutic composition according to claim 1, wherein said RNA is a RNA-guide complexed with a RNA-guided endonuclease protein.

8) A therapeutic composition according to any one of claims 1 to 7, wherein at least two different endonuclease reagents are encapsulated under RNA form into the particles.

9) A therapeutic composition according to claim 8, wherein said different endonuclease reagents are at least a RNA encoding a RNA-guided endonuclease and a guide RNA.

10) A therapeutic composition according to claim 4 wherein said core hydrophobic domain is a biodegradable conjugate of hydrophobic monomers.

11) A therapeutic composition according to claim 4, wherein said core hydrophobic and proximal polar domains are covalently linked.

12) A therapeutic composition according to claim 4, wherein said core hydrophobic and proximal polar domains are linked by peptide linkers.

13) A therapeutic composition according to claim 10, wherein said hydrophobic monomers conjugate are of aminolipids, such as ionized cationic lipid 1,2-dilinoleyloxy-3-dimethylaminopropane (DLinDMA).

14) A therapeutic composition according to claim 13, wherein said aminolipids are mixed with PEG-lipids to form said polar domain.

15) A therapeutic composition according to claim 13 or 14, wherein said aminolipids allows binding of ApoE in-vivo, said ApoE facilitating Apo E mediated endocytosis.

16) A therapeutic composition according to any one of claims 1 to 15, wherein said endonuclease reagent endocytosis is mediated via a receptor of the LDL or VLDL receptor family.

17) A therapeutic composition according to any one of claims 1 to 16, wherein said at least one polar domain is poly-N,N-di(C1-C6)alkyl-amino(C1-C6)alkyl-ethacrylate, poly-N,N-di(C1-C6)alkyl-amino(C1-C6)alkyl-methacrylate, or poly-N,N-di(C1-C6)alkyl-amino(C1-C6)alkyl-acrylate, or a combination thereof.

18) A therapeutic composition according to claim 17, wherein said hydrophobic monomers include at least (C2-C8)alkyl-ethacrylate, a (C2-C8)alkyl-methacrylate, or a (C2-C8)alkyl-acrylate.

19) A therapeutic composition according to any one of claims 1 to 18, wherein said hydrophobic monomers conjugate are mixed with carboxylic acid monomers and tertiary amino monomers.

20) A therapeutic composition according to any one of claims 1 to 19, wherein said at least one polar domain is linked to a targeting domain.

21) A therapeutic composition according to claim 20, wherein said targeting domain comprises ScFv of an antibody targeting a cell surface antigen.

22) A therapeutic composition according to claim 21, wherein said cell surface antigen is a protein is selected from a LDL, VLDL receptors or cell surface heparin sulfate proteoglycans.

23) A therapeutic composition according to any one of claims 1 to 22, wherein said at least one polar domain is linked to a N-acetylgalactosamine ligand.

24) A therapeutic composition according to any one of claims 20, 21 and 23, wherein said targeting domain is a ligand of asiaglycoprotein.

25) A therapeutic composition according to claim 4, wherein said particles encapsulating said endonuclease reagent are of 50 to 90 nm diameter range.

26) A biodegradable delivery capsule for gene targeting of a cell in-vivo, characterized in that an endonuclease reagent, preferably under RNA form is complexed with (1) at least one polar domain, which is linked to biodegradable conjugate(s) of hydrophobic monomers to form spherical particles of 50 to 100 nm diameter range and (2) and antiviral compound, which has no endonuclease activity.

27) A biodegradable delivery capsule according to claim 26, wherein said antiviral compound is selected from:
Inhibitor of sodium taurocholate cotransporting polypeptide (NTCP), such as Myrcludex;
cccDNA inhibitor, such as disubstituted sulfonamide (DSS) compounds, antibodies inducing Lymphotoxin beta receptor activation or LTβR agonists;
RNAi or compounds aiming at reducing HBV genes expression, in particular Helioxanthin or Ethanol extract from Ampelopsis sinica root;
La protein inhibitor, such as HBSC11;
Capsid allosteric modulators, such as ABI H0731, JNJ 56136379 (or JNJ379), Morphothiadine (GLS4), NVR 3 778 or NVR1221;
Reverse transcriptase inhibitors, in particular nucleoside analogs, such as Lamivudine, Telbivudine, Entecavir, Adefovir, Tenofovir, Besifovir, MIV-210, MCC-478 or Alamifovir;
Inhibition of HBsAg release, such as REP2139 and GC1102;
Immunoregulators, such as Thymosin-α1;
Vesatolimod (GS-9620);
Vaccines, such as GS-4774, ABX-203, TG-1050, INO-1800; FP-02.2; and
Immune stimulators, such as SB-9200, AIC649, Cellular inhibitor of apoptosis proteins (cIAPs) Cytokines: IL-21 or recombinant human IL-7 and antibodies antagonist of Immune checkpoint (Nivolumab and Pembrolizumab).

28) A biodegradable delivery capsule according to claim 26 or 27, wherein at least two RNA endonuclease reagents are included into said spherical particles.

29) A biodegradable delivery capsule according to any one of claims 26 to 28, wherein said biodegradable matrix comprises at least two polar domains, such that the inner core particle is hydrophilic.

30) A biodegradable delivery capsule according to claim 29, wherein said hydrophilic inner core particle encapsulates said antiviral compound.

31) A biodegradable delivery capsule according to claim 30, wherein said further endonuclease reagent comprises a polypeptide.

32) A biodegradable delivery capsule according to claim 31, wherein said polypeptide encodes a RNA or DNA guided endonuclease.

33) A pharmaceutical composition comprising a biodegradable delivery capsule according to any one of claims 26 to 32 with a pharmaceutically acceptable medium.

34) A pharmaceutical composition according to claim 33, for use as a medicament.

35) A pharmaceutical composition according to claim 33 or 34, for use in the treatment of a liver disease.

36) A pharmaceutical composition according to claim 33 or 34, for use in the treatment of an infectious disease.

37) A pharmaceutical composition according to claim 35 or 36, wherein said disease is a viral disease such as hepatitis.
